(19) United States
(12) Patent Application Publication
(10) Pub. No.: US 2023/0257746 A1
(43) Pub. Date: Aug. 17, 2023

(54) TARGETING ZNF410 FOR FETAL HEMOGLOBIN INDUCTION IN BETAHEMOGLOBINOPATHIES

(71) Applicant: The Children's Medical Center Corporation, Boston, MA (US)

(72) Inventors: Daniel E. BAUER, Cambridge, MA (US); Divya VINJAMUR, Boston, MA (US)

(73) Assignee: The Children's Medical Center Corporation, Boston, MA (US)

(21) Appl. No.: 18/016,190

(22) PCT Filed: Jul. 19, 2021
(86) PCT No.: PCT/US2021/042152

§ 371 Date: Jan. 13, 2023

Related U.S. Application Data
(60) Provisional application No. 63/053,308, filed on Jul. 17, 2020.
(60) Provisional application No. 63/068,150, filed on Aug. 20, 2020.

(51) Int. Cl. C12N 15/113; C07K 16/18; A61K 38/46; A61K 31/7088; A61K 31/175; A61K 31/166; A61K 31/352; A61K 31/255; A61K 31/433; A61P 7/06

(52) U.S. Cl. CPC C12N 15/113; C07K 16/18; A61K 38/465; A61K 31/7088; A61K 31/175; A61K 31/166; A61K 31/352; A61K 31/255; A61K 31/433; A61P 7/06; C12N 2310/11; C12N 2310/127; C12N 2310/12; C12N 2310/141; C12N 2310/531; C12N 2310/16

(57) ABSTRACT

Provided herein are methods and compositions related to treating a hemoglobinopathy. Also provided herein a gene therapeutics for the treatment of hemoglobinopathies and the induction of fetal hemoglobin (HbF).

### CROSS-REFERENCE TO RELATED APPLICATIONS

This application claims benefit under 35 U.S.C. § 119(d) of the U.S. Provisional Application No. 63/053,308 filed Jul. 17, 2020 and U.S. Provisional Application No. 63/068,150 filed Aug. 20, 2020, the contents of both of which are incorporated herein by reference in their entirety.

### GOVERNMENT SUPPORT

This invention was made with government support under Grant No. DP2HL137300 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 15, 2021, is named 701039-098070WOPT_SL.txt and is 86,920 bytes in size.

### TECHNICAL FIELD

The technology described herein relates to hemoglobinopathies.

### BACKGROUND

Hemoglobinopathies encompass a number of anemias in which there is a decreased production and/or increased destruction of red blood cells (RBCs). Hemoglobinopathies can also include genetic defects that result in the production of abnormal hemoglobins with a concomitant impaired ability to maintain oxygen concentration. These disorders are associated with the β-globin protein and are referred to generally as β-hemoglobinopathies. There is a clinically unmet need for therapeutics for the most common monogenic diseases that are also safe and well tolerated in subjects.

### SUMMARY

The compositions and methods provided herein are based, in part, on the discovery of zinc finger protein 410 (ZNF410) as a repressor of fetal hemoglobin (HbF) expression and novel compositions capable of decreasing the levels and activity of ZNF410 for the treatment of hemoglobinopathies that can be well tolerated across a diverse set of cellular contexts.

In one aspect, provided herein is a method of increasing fetal hemoglobin level in a subject. Generally, the method comprises decreasing the level or activity of ZNF410 in the subject. For example, by administering to the subject an agent that decreases the level or activity of ZNF410. In some embodiments, the subject has a hemoglobinopathy.

Accordingly, in another aspect, provided herein is a method of treating a hemoglobinopathy in a subject. Generally, the method comprises decreasing the level or activity of ZNF410 in the subject. For example, by administering to the subject an agent that decreases the level or activity of ZNF410.

In some embodiments of any one of the aspects, the agent that decreases the level or activity of ZNF410 is a nucleic acid comprising a nucleotide sequence complementary to at least a portion of a nucleic acid encoding ZNF410, an anti-ZNF410 antibody, a zinc finger inhibitor, or a dominant negative ZNF410 polypeptide.

In some embodiments of any one of the aspects, the hemoglobinopathy is a β-hemoglobinopathy. In some embodiments of any one of the aspects, the hemoglobinopathy is selected from the group consisting of: sickle cell disease; sickle cell anemia; sickle-hemoglobin C disease (HbSC); sickle beta-plus-thalassemia (HbS/β+); sickle beta-zero-thalassemia (HbS/β0); and β-thalassemia.

In some embodiments of any of the aspects, level or activity of ZNF410 is decreased by at least 50%, at least 60%, at least 70%, at least 80%, at least 90%, or more as compared to an appropriate control.

In some embodiments of any of the aspects, a decreased level or activity of ZNF410, in a cell of the subject, increases the level and/or activity of fetal hemoglobin (HbF) by at least 50%, at least 60%, at least 70%, at least 80%, at least 90%, or more as compared to an appropriate control.

It is noted that the subject can be a mammal, e.g., a human.

In another aspect, provided herein is a synthetic nucleic acid molecule capable of targeting zinc finger 410 (ZNF410), the nucleic acid molecule comprising: a nucleotide sequence selected from the group consisting of SEQ ID NO: 1-SEQ ID NO: 183.

In another aspect, provided herein is a composition, e.g., a gene editing composition comprising: (a) a synthetic nucleic acid molecule comprising a nucleotide sequence selected from the group consisting of SEQ ID NO: 1-SEQ ID NO: 183; and (b) a nuclease enzyme.

In some embodiments of any of the aspects, the synthetic nucleic acid molecule comprises a nucleotide sequence selected from the group consisting of: SEQ ID NO: 1-SEQ ID NO: 169. In yet some

In some embodiments of any of the aspects, the synthetic nucleic acid molecule comprises the nucleotide sequence GTACAGTTGAAGGTTGTGAC (SEQ ID NO: 19).

In yet another aspect, provided herein is a vector comprising the synthetic nucleic acid molecule provided herein. In some embodiments, the vector further comprises a polynucleotide encoding a nuclease enzyme.

In another aspect, provided herein is a nanoparticle comprising the synthetic nucleic acid molecule provided herein. In some embodiments, the nanoparticle further comprises a nuclease enzyme.

In still another aspect, provided herein is a pharmaceutical composition comprising: (a) the gene editing composition provided herein; or (b) the vector provided herein; or (c) the nanoparticle provided herein; and (d) a pharmaceutically acceptable carrier.

### BRIEF DESCRIPTION OF THE DRAWINGS

This application file contains at least one drawing executed in color. Copies of this patent application publication with color drawings will be provided by the Office upon request and payment of the necessary fee.

FIGS. 1A-1E show ZNF410 is a novel repressor of fetal hemoglobin (HbF) expression. FIG. 1A shows a schematic representation of CRISPR/Cas9-based knockout screen in HUDEP-2 cells to identify novel repressors of HbF expression. FIG. 1B shows the gene ZNF410 was prioritized for further study based on enrichment of single guide RNAs (sgRNAs) targeting ZNF410 in HbF+ cells with neutral fitness score. The results of the screen were independently validated in HUDEP-2 cells using lentiviral delivery of Cas9 and sgRNA targeting ZNF410. Targeted HUDEP-2 cells were differentiated to orthochromatic erythroblasts and assayed on day 9 of differentiation. FIG. 1C shows there was an increase in HbF+ cells by intra-cellular HbF staining, HBG expression by RT-qPCR and HbF level by HPLC in ZNF410-targeted cells compared to non-targeting (NT) and Mock control HUDEP-2 cells. FIG. 1D shows three ZNF410 knockout HUDEP-2 clones show an increase in the percentage of HbF+ cells by intra-cellular staining (white bars) compared to wild type HUDEP-2 cells. When ZNF410 was re-expressed in these null clones, there was a reduction in the percentage of HbF+ cells by intra-cellular staining in all three clones (gray bars), indicating that ZNF410 is a bona fide HbF repressor. FIG. 1E shows ZNF410 was targeted by RNP electroporation of Cas9 protein and sgRNA in CD34+ HSPCs from mobilized adult peripheral blood of 4 individual donors and cells were subsequently differentiated to erythroid cells in vitro. Robust indel efficiency was achieved at ZNF410 4 days after electroporation. At the end of erythroid culture, normal erythroid maturation was observed in ZNF410 edited samples compared to Mock control samples by staining for erythroid cell surface markers CD71 and CD235a and by assaying the percentage of enucleation by Hoechst staining. There was an increase in the percentage of HbF in ZNF410 edited primary erythroid cells compared to Mock control cells as measured by hemoglobin HPLC. These results show that targeting ZNF410 does not impact erythroid maturation.

FIGS. 2A-2E show ZNF410 binds to DNA at two CHD4-upstream regulatory sites on chr12 via densely clustered motifs. FIG. 2A shows dense mutagenesis of ZNF410 protein-coding sequence (1-478 amino acids) was performed using 183 gRNAs (restricted by NGG protospacer adjacent motif for SpCas9) targeting ZNF410 to identify effective gRNAs and regions of the gene that are important for HbF regulation by determining HbF-enrichment and cell fitness scores for all sgRNA-targetable regions. Each dot represents an sgRNA. Black line is Loess regression curve across all data points. Positive scores indicate HbF induction. The 5 C2H2 zinc-finger domains (red rectangles) of ZNF410 are especially critical for HbF regulation in that mutating these sites gives robust induction of HbF. FIG. 2B shows genome-wide ZNF410 binding was identified by CUT&RUN using anti-HA antibody in 4 HUDEP-2 samples with ZNF410-HA over-expression compared to 6 IgG control samples. Tremendous enrichment for ZNF410 binding was identified only at 2 sites in the genome—at the promoter of CHD4 and at an enhancer ˜6 kb upstream of CHD4 (CHD4—6 kb enhancer). FIG. 2C shows genome-wide ZNF410 motif occurrences were mapped using the pwmscan webtool and a 3 kb sliding window was used to identify regions of the genome with multiple binding sites. The motif shown in the center of the graph was obtained from the JASPAR database. Large clusters of ZNF410 motifs were found only at 3 locations in the genome. Two of these corresponded to the CHD4 promoter and CHD4-6 kb enhancer ZNF410 binding sites identified by CUT&RUN. The third cluster is in the intron of the GALNT18 gene (E), which is not expressed in erythroid cells and these sequences are neither bound by ZNF410 nor inaccessible chromatin. FIG. 2D shows the CHD4 locus on chr12 either zoomed out (top panel) or zoomed in on each motif cluster (bottom panels) showing ZNF410 binding at the CHD4 promoter and CHD4—6 kb enhancer regions in a representative control (IgG) and combined ZNF410-HA samples (red peaks), positions of ZNF410 motifs (red rectangles), single locus footprinting (red bars), open chromatin regions mapped using ATAC-seq (gray peaks) and DNA sequence conservation using phyloP. FIG. 2E shows the next highest enrichment for ZNF410 binding was observed in an NBPF19 intron at a much lower level than at the CHD4 locus.

FIGS. 3A-3G show ZNF410 represses HbF through positive regulation of CHD4 expression. FIG. 3A shows differential gene expression analysis of ZNF410 targeted cells compared to control shows that CHD4 is the most significantly down-regulated gene. FIG. 3B shows comparison of genes upregulated in ZNF410 and CHD4 mutant cells by GSEA shows enrichment for CHD4 regulated genes in the ZNF410 regulated gene set. FIG. 3C shows Pearson correlation between ZNF410 dependency and CHD4 dependency in 558 cell lines with genome-wide CRISPR screens identifies CHD4 as the most ZNF410 codependent gene across cell lines. FIG. 3D shows CHD4 expression relative to Catalase expression measured by RT-qPCR in ZNF410 targeted HUDEP-2 cells and primary erythroblasts derived from CD34+ HSPCs. FIG. 3E shows paired cleavages by Cas9 generated an element deletion clone (CHD4Δ6.7 kb), with the biallelic deletion spanning both of the ZNF410 binding regions upstream of CHD4. FIG. 3F shows CHD4 expression relative to Catalase expression measured by q-RT-PCR in the CHD4Δ6.7 kb clone compared to HUDEP-2 cells. HBG expression relative to total β-like globin (HBG+HBB) was measured by RT-qPCR in the CHD4Δ6.7 kb deletion clone compared to HUDEP-2 control cells. CHD4Δ6.7 kb clones and HUDEP-2 cells were subjected to negative control and ZNF410 targeting using RNP electroporation. FIG. 3G shows CHD4 expression relative to Catalase expression measured by RT-qPCR shows no further change in CHD4 expression in the element deletion clone with ZNF410 targeting indicating that ZNF410 regulated CHD4 through this DNA element. HBG expression measured by RT-qPCR in the CHD4Δ6.7 kb clone shows no further increase upon ZNF410 targeting indicating that the entire role of ZNF410 in HbF regulation is through regulation of CHD4 expression.

FIGS. 4A-4D show 4410 knockout mice are viable and have normal hematology. Mouse ES cells heterozygous for the mouse gene 4410 (which is the mouse ortholog of human ZNF410) gene-trap allele (Gt), obtained from EuMMCR, were used to generate heterozygous (4410+/Gt) and homozygous (4410 Gt/Gt) gene-trap mice. FIG. 4A shows mice of various genotypes including homozygous Zfp410 mutants were born at expected Mendelian ratios; data is from n=11 mice from 2 litters. FIG. 4B shows 4410 expression was diminished in Zfp410 Gt/Gt mouse peripheral blood compared to heterozygous (*=p-value<0.05) and wildtype control animals, confirming successful Zjp410 targeting. FIG. 4C shows mouse weight was measured at various time points over the course of 15 weeks. FIG. 4D shows CBC analysis was performed to assess hematological parameters using peripheral blood on the Advia instrument; Total hemoglobin (HGB), mean corpuscular volume (MCV), reticulocyte count, white blood cell (WBC) count, neutrophil count and platelet count are within the normal range (dotted lines). Values for the normal range of various hematological parameters for C57BL/6 mice were obtained from the Charles River website.

FIGS. 5A-5J show xenografts of ZNF410 gene edited primary human HSPCs show normal reconstitution of various hematopoietic lineages in NBSGW immunodeficient mice with elevated HbF level in vivo. FIG. 5A shows an experimental schematic of gene editing and transplant of human CD34+ HSPCs in immune-deficient NBSGW mice. Animals were euthanized 16 weeks post-transplant and bone marrow (BM) was harvested and sorted into various subpopulations by flow cytometry. In panels B-E, cells of two independent CD34+ HSPC donors were edited and transplanted into 6 mice for each condition (Mock or ZNF410). Each point on the graph represents one mouse and each color represents a different donor. FIG. 5B shows indel frequency at ZNF410 was quantified in input cells at the time of transplant, in engrafted cells at the time of harvest and in sorted hematopoietic subpopulations. The percentage of frameshift alleles is represented in gray and the percentage of in-frame alleles is represented in white for each bar. FIG. 5C shows hemolysates of bone marrow erythroid cells were used to assess HbF by hemoglobin HPLC. ZNF410 edited HSPCs showed similar (FIG. 5D) engraftment and (FIG. 5E) multilineage hematopoietic reconstitution as unedited controls. In panels (F-J), CD34+ HSPCs from a third human donor were edited at either ZNF410, BCL11A or ZBTB7A and transplanted in NBSGW mice; each point represents one mouse. FIG. 5F shows indel frequency at ZNF410, BCL11A and ZBTB7A was quantified in input cells at the time of transplant, in engrafted cells at the time of harvest and in sorted hematopoietic subpopulations. The percentage of frameshift alleles is represented in gray and the percentage of in-frame alleles is represented in white for each bar. FIG. 5G shows comparison of % HbF in ZNF410 (n=4), BCL11A (n=3) and ZBTB7A (n=3) edited and Mock control (n=4) engrafted erythroid cells by hemoglobin HPLC. FIG. 5H shows comparison of engraftment in xenografts of ZNF410, BCL11A and ZBTB7A edited and Mock control CD34+ HSPCs. FIG. 5I, CHD4 expression was measured by RT-qPCR in human erythroid cells from Mock (n=4) and ZNF410 edited (n=4) xenografts. FIG. 5J shows comparison of multilineage hematopoietic reconstitution in xenografts of ZNF410, BCL11A and ZBTB7A edited and Mock control CD34+ HSPCs. These data show that deficiency of ZNF410 in human HSPCs is tolerated with retained hematopoietic function as measured by self-renewal and multilineage differentiation in vivo as well as normal erythroid reconstitution with elevated HbF level.

FIGS. 6A-6D show ZNF410 repression of fetal hemoglobin level is independent of BCL11A. The role of ZNF410 in HbF repression was assessed in comparison to known HbF repressors, BCL11A and ZBTB7A, in primary human CD34+ hematopoietic stem and progenitor cell (HSPC) derived erythroblasts. FIG. 6A shows efficient editing was observed at all targeted loci on day 4 of erythroid culture. Editing was also measured at the end of erythroid culture on Day 18 to assess maintenance of edits. FIG. 6B shows erythroid maturation, based on CD71 and CD235a expression, and FIG. 6C shows enucleation were assessed on day 18 of erythroid culture. FIG. 6D shows % HbF was measured by HPLC on day 18 of erythroid culture. ZNF410 appears to repress HbF independently from known HbF repressors like BCL11A.

FIGS. 7A-7B show ZNF410 chromatin occupancy. ZNF410 binding in a representative control (IgG) and combined ZNF410-HA samples, positions of ZNF410 motifs, open chromatin regions mapped using ATAC-seq (gray peaks) and DNA sequence conservation using phyloP. FIG. 7A shows enrichment for ZNF410 binding at an NBPF19 intron displayed at the same peak resolution as the CHD4 locus. FIG. 7B shows ZNF410 does not bind to the γ-globin gene cluster.

FIGS. 8A-8D show ZNF410 represses HbF by positively regulating CHD4. FIG. 8A shows the comparison of genes downregulated in ZNF410 and CHD4 mutant cells by GSEA shows a high enrichment for CHD4 regulated genes in the ZNF410 regulated gene set.

FIG. 8B shows the expression of ZNF410 and CHD4 are significantly correlated across 54 human tissues from the Gtex database in addition to the clone shown in FIG. 3. FIG. 8C shows CHD4 expression relative to Catalase expression measured by RT-qPCR in CHD4Δ6.7 kb clones compared to HUDEP-2 cells. FIG. 8D shows CHD4Δ6.7 kb clones and HUDEP-2 cells were subjected to AAVS1 (negative control), ZNF410 and ZBTB7A targeting using RNP electroporation. Editing efficiency measured by indel frequency in HUDEP-2 cells and CHD4Δ6.7 kb clones targeted with ZNF410 or ZBTB7A sgRNAs. Shaded portion of bar represents percentage of indels resulting in frameshift alleles. White portion of bar represents in-frame indels.

FIG. 9 shows a 4410 gene trap allele. A targeting cassette with a splice acceptor site upstream of the LacZ gene is inserted in intron 5 thus disrupting expression of full-length Zfp410.

FIGS. 10A-10D show ZNF410 represses fetal hemoglobin expression independent of BCL11A repression in primary human erythroblasts. The role of ZNF410 in HbF repression was assessed in comparison to known HbF repressors, BCL11A and ZBTB7A, in primary human CD34+ hematopoietic stem and progenitor cell (HSPC) derived erythroblasts. FIG. 10A shows efficient editing was observed at all targeted loci on day 4 of erythroid culture. FIG. 10B shows % HbF was measured by HPLC on day 18 of erythroid culture. FIG. 10C shows Erythroid maturation, based on CD71 and CD235 expression, and FIG. 10D shows enucleation assessed on day 18 of erythroid culture. These results show that targeting ZNF410 does not impact erythroid maturation. Furthermore, ZNF410 appears to repress HbF independently from known HbF repressors like BCL11A.

FIGS. 11A-11D show the Indel frequency in xenografts. FIG. 11A shows frequency of indels obtained at ZNF410 in xenograft samples from Input (donor CD34+ cells assayed 4 days after editing), Engrafted (human cells within total mouse bone marrow) and sorted CD34+, CD235A+, CD19+ and CD3+ cells. Each dot represents an individual mouse.

FIG. 11B show frequency of indels obtained at ZNF410, BCL11A and ZBTB7A in xenograft samples from Input (white bars) and Engrafted (gray bars) cells. FIG. 11C shows frequency of frameshifts observed in indels obtained at ZNF410, BCL11A and ZBTB7A in xenograft samples from Input (white bars) and Engrafted (gray bars) cells. Together with the results in FIG. 5, these data show that targeting ZNF410 in human hematopoiesis is well tolerated unlike targeting other HbF repressors like BCL11A or ZBTB7A.

### DETAILED DESCRIPTION

The compositions and methods provided herein are based, in part, on the discovery of zinc finger protein 410 (ZNF410) as a repressor of fetal hemoglobin (HbF) expression and compositions capable of decreasing the levels of ZNF410 for the treatment of hemoglobinopathies. The compositions provided herein can be well tolerated across a diverse set of cellular contexts.

In one aspect, provided herein are compositions that reduce or decrease the level and/or activity of ZNF410 in a cell or a subject.

In another aspect, the compositions provided herein can be used for the treatment of a hemoglobinopathy in a subject. In another aspect, the compositions provided herein increase the level and/or activity of fetal hemoglobin (HbF).

Embodiments of the various aspects described herein include an agent that decreases the level or activity of ZNF410. It is noted that an agent can be selected from small organic or inorganic molecules; saccharides, oligosaccharides, polysaccharides, amino acids, peptides, polypeptides, peptidomimetic, nucleotides and nucleosides, oligonucleotides, polynucleotides, nucleic acid analog analogs and derivatives, biological macromolecules, and extracts made from biological materials such as bacteria, plants, fungi, or mammalian cells. It is noted that the agent that decreases the level or activity of ZNF410 can be a naturally occurring molecule or a synthetic molecule.

In some embodiments of any one of the aspects, the agent that decreases the level or activity of ZNF410 is a nucleic acid, for example, a nucleic acid molecule comprising a nucleotide sequence complementary to at least a portion of a nucleic acid encoding ZNF410. Sequences for nucleic acid encoding ZNF410 are well known in the art. Exemplary sequences for ZNF410 can be found in Genebank with Accession Numbers as listed NM 001242924.2 (SEQ ID NO: 186), NM 021188.3 (SEQ ID NO: 187), NM 001242926.2 (SEQ ID NO: 190), NM 001242927.2 (SEQ ID NO: 192), and NM 001242928.2 (SEQ ID NO: 193).

In some embodiments of any one of the aspects, the nucleic acid agent that decreases the level or activity of ZNF410 is a guide RNA, a sgRNA, an siRNA, a shRNA, an antisense oligonucleotide, an aptamer, a ribozyme, a DNAzyme, or a microRNA. In some embodiments of any one of the aspect, the nucleic acid agent that decreases the level or activity of ZNF410 comprises a nucleotide sequence selected from the group consisting of SEQ ID NO: 1-SEQ ID NO: 183. For example, the nucleic acid agent that decreases the level or activity of ZNF410 comprises a nucleotide sequence selected from the group consisting of SEQ ID NO: 1-SEQ ID NO: 169. In some preferred embodiments, the nucleic acid agent that decreases the level or activity of ZNF410 comprises the nucleotide sequence GTACAGTTGAAGGTTGTGAC (SEQ ID NO: 19).

It is noted that the nucleic acid agent can be single-stranded, double-stranded, partially double-stranded. Further, the nucleic acid agent can comprise additional nucleic acid structures. For example, the nucleic acid agent can comprise hairpin structure, a mismatch in a double-stranded region, and the like.

In some embodiments of any one of the aspects, the agent that decreases the level or activity of ZNF410 is an anti-ZNF410 antibody. Anti-ZNF410 antibodies are known in the art and commercially available. For example, the anti-ZNF410 antibodies available from Sigma-Aldrich (cat #HPA002871, SAB2104187 and SAB1407818), the anti-ZNF410 antibodies available from Novus Biologicals (cat #NBP2-21008), from antibodies-online.com (cat #ABIN2777798, ABIN931225, ABIN1882025, ABIN2155007, ABIN5535398, ABIN6742349, ABIN528331, ABIN2687242 and ABIN5516407), ZNF410 Polyclonal antibody from Invitrogen, ZNF410 antibody N1C1 from GeneTex, the anti-ZNF410 antibodies available from MyBioSource.com (cat #MBS8501808 and MBS9125687), Proteintech #14529-1-AP and RRID:AB_2257520.

In some embodiments of any one of the aspects, the agent that the agent that decreases the level or activity of ZNF410 is a zinc finger inhibitor. Generally, zinc finger inhibitors are substances or compounds that interact adversely with zinc fingers and cause them to release their zinc from its binding site, disrupting the conformation of the polypeptide chain and rendering the zinc fingers, e.g., ZNF410 ineffective, thereby preventing them from performing their associated cellular functions. This is typically accomplished through chelation of the zinc binding site. Accordingly, in some embodiments, the agent that the agent that decreases the level or activity of ZNF410 is a zinc finger ejector compound. Exemplary zinc finger ejector compounds are described in US Patent Publication US20080039528, contents of which are incorporated herein by reference in their entirety. In some embodiments of any one of the aspect, the zinc finger ejector compound is selected from the group consisting of azodicarbonamide (ADA), 3-nitrosobenzamide (NOBA), 6-nitroso-1,2-benzopyrone (NOBP), 2,2′-di-thiobisbenzamide (DIBA), mercaptobenzamides, Pyridinioalkanoyl thiolesters (PATES), and bis-thiadizolbenzene-1,2-diamine.

In some embodiments of any one of the aspects, the agent that decreases the level or activity of ZNF410 is a dominant negative ZNF410 polypeptide.

Hemoglobinopathies

Fetal hemoglobin (HbF) is a tetramer of two adult α-globin polypeptides and two fetal β-like γ-globin polypeptides. During gestation, the duplicated γ-globin genes constitute the predominant genes transcribed from the β-globin locus. Following birth, γ-globin becomes progressively replaced by adult β-globin, a process referred to as the “hemoglobin switch” (3). The molecular mechanisms underlying this switch have remained largely undefined and have been a subject of intense research. The developmental switch from production of predominantly fetal hemoglobin or HbF (α₂γ₂) to production of adult hemoglobin or HbA (α₂β₂) begins at about 28 to 34 weeks of gestation and continues shortly after birth at which point HbA becomes predominant. This switch results primarily from decreased transcription of the gamma-globin genes and increased transcription of beta-globin genes. On average, the blood of a normal adult contains only about 2% HbF, though residual HbF levels have a variance of over 20 fold in healthy adults (Atweh, Semin. Hematol. 38(4):367-73 (2001)).

Hemoglobinopathies encompass a number of anemias of genetic origin in which there is a decreased production and/or increased destruction (hemolysis) of red blood cells (RBCs). Symptoms typically associated with a hemoglobinopathy, include for example, anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris.

These disorders also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders specifically associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38(4):367-73 (2001)).

For β-hemoglobinopathies, such as sickle cell anemia and the β-thalassemias, the disease symptoms can be ameliorated by increased HbF production. (Reviewed in Jane and Cunningham Br. J. Haematol. 102: 415-422 (1998) and Bunn, N. Engl. J. Med. 328: 129-131 (1993)). For example, such compounds as 5-azacytidine, hydroxyurea, recombinant human erythropoietin, and butyric acid analogs, as well as combinations of these agents have been used for the treatment of hemoglobinopathies.

However, varying drawbacks have contraindicated the long term use of such agents or therapies, including unwanted side effects and variability in patient responses. For example, while hydroxyurea stimulates HbF production and has been shown to clinically reduce sickling crisis, it is potentially limited by myelotoxicity and the risk of carcinogenesis. Potential long term carcinogenicity would also exist in 5-azacytidine-based therapies. Erythropoietin-based therapies have not proved consistent among a range of patient populations. The short half-lives of butyric acid in vivo have been viewed as a potential obstacle in adapting these compounds for use in therapeutic interventions. Furthermore, very high dosages of butyric acid are necessary for inducing γ-globin gene expression, requiring catheritization for continuous infusion of the compound. Moreover, these high dosages of butyric acid can be associated with neurotoxicity and multiorgan damage (Blau, et al., Blood 81: 529-537 (1993)). While even minimal increases in HbF levels are helpful in sickle cell disease, β-thalassemias require a much higher increase that is not reliably, or safely, achieved by any of the currently used agents (Olivieri, Seminars in Hematology 33: 24-42 (1996)).

Identifying regulators of HbF induction and production provide a means to devise therapeutic interventions that overcome the various drawbacks of the compounds described above.

The methods and compositions provided herein are for use in the treatment of hemoglobinopathies and the induction of fetal hemoglobin in a subject.

Zinc Finger 410 (ZNF410)

ZNF410 (also known as APA-1) is a transcription factor that activates the transcription of matrix-remodeling genes such as MMP1 during fibroblast senescence. See, e.g., Benanti et al., Mol. Cell. Biol. 22:7385-7397 (2002), which is incorporated herein by reference in its entirety. Sequences of ZNF410 are known in the art, e.g., NCBI GeneID: 57862. For example, the human gene sequence, mRNA transcript sequences, and amino acid sequences of ZNF410 are provided herein as SEQ ID NOs: 185-191 below in Table 1.

TABLE 1

| SEQ ID NO: | NCBI Reference | Description |
|---|---|---|
| SEQ ID NO: | NC_000014.9: | ZNF410 Gene and reference sequence- |
| 185 | 73886859- | Homo sapiens chromosome 14, |
| | 73932521 | GRCh38.p13 Primary Assembly |
| SEQ ID NO: | NCBI Reference | Homo sapiens zinc finger protein 410 |
| 186 | Sequence: | (ZNF410), transcript variant 1, mRNA |
| | NM_001242924.2 | |
| SEQ ID NO: | NCBI Reference | Homo sapiens zinc finger protein 410 |
| 187 | Sequence: | (ZNF410), transcript variant 2, mRNA |
| | NM_021188.3 | |
| SEQ ID NO: | NCBI Reference | zinc finger protein 410 isoform a [Homo |
| 188 | Sequence: | sapiens] |
| | NP_001229853.1 | |
| SEQ ID NO: | NCBI Reference | zinc finger protein 410 isoform b [Homo |
| 189 | Sequence: | sapiens] |
| | NP_067011.1 | |
| SEQ ID NO: | NCBI Reference | Homo sapiens zinc finger protein 410 |
| 190 | Sequence: | (ZNF410), transcript variant 3, mRNA |
| | NM_001242926.2 | |
| SEQ ID NO: | NCBI Reference | zinc finger protein 410 isoform c [Homo |
| 191 | Sequence: | sapiens] |
| | NP_001229855.1 | |

As disclosed herein in the working examples, ZNF410 is a novel fetal hemoglobin (HbF) repressing transcription factor that is dispensable for erythroid maturation. ZNF410 does not bind directly to the γ-globin genes. The predominant site of chromatin occupancy is CHD4, encoding the NuRD nucleosome remodeler, itself required for HbF repression. See, e.g., Sankaran, V. G. et al. Human Fetal Hemoglobin Expression Is Regulated by the Developmental Stage-Specific Repressor BCL11A. Science 322, 1839-1842 (2008); Masuda, T. et al. Transcription factors LRF and BCL11A independently repress expression of fetal hemoglobin. Science 351, 285-289 (2016); and Sher, F. et al. Rational targeting of a NuRD subcomplex guided by comprehensive in situ mutagenesis. Nat. Genet. 51, 1149-1159 (2019), incorporated by reference in their entireties.

As provided herein, CHD4 has two ZNF410-bound regulatory elements with 27 combined ZNF410 binding motifs constituting unparalleled genomic clusters. Knockout of ZNF410 reduces CHD4 enough to substantially de-repress HbF while avoiding the cellular toxicity of complete CHD4 loss. Mice with constitutive deficiency of the homolog Zfp410 are born at expected Mendelian ratios with unremarkable hematology. Furthermore, ZNF410 is unnecessary for human hematopoietic engraftment potential and erythroid maturation unlike known HbF repressors (e.g., BCL11A).

The synthetic nucleic acid molecules, gene editing compositions, and agents described herein target zinc finger protein 410 (ZNF410). In particular, the agents, nucleic acid molecules, and gene editing compositions provided herein can decrease the level or activity of ZNF410.

As used herein, the term “decreasing the level of ZNF410” in a cell or a subject indicates that ZNF410 polypeptide or nucleic acids encoding the ZNF410 polypeptide are at least 5% lower in a treated cell, subject, or population thereof with any of the synthetic nucleic acid molecules, agents, or gene editing compositions provided herein than in a comparable, control cell, control/healthy subject, or population thereof that is not treated with the synthetic nucleic acid molecules, agents, or gene editing compositions provided herein.

As used herein, the term “decreasing the activity of ZNF410” in a cell or a subject indicates that ZNF410 function described herein is at least 5% lower in a treated cell, subject, or population thereof with any of the synthetic nucleic acid molecules, agents, or gene editing compositions provided herein compared with an appropriate control.

As used herein, the term “increasing the fetal hemoglobin levels” in a cell or a subject indicates that fetal hemoglobin is at least 5% higher in a cell, subject, or population thereof treated with any one of the synthetic nucleic acid molecules, agents, or gene editing compositions provided herein than in a comparable, control cell, control subject, or population thereof that is not treated with the synthetic nucleic acid molecules, agents, or gene editing compositions provided herein.

The levels of ZNF410 or fetal hemoglobin (HbF) can be determined by methods known in the art. For example, PCR, Western blotting, immunological methods, flow cytometric analyses, ELISA. Accordingly, the activity of ZNF410 can be determined by methods known in the art, e.g., a chromatin occupancy assay, binding assays, pull-down assays, RT-PCR of fetal hemoglobin levels, animal models, etc. ZNF410 activity can be assayed by measuring fetal hemoglobin expression at the mRNA or protein level following treatment with a synthetic nucleic acid molecule, agent, or gene editing system provided herein.

In some embodiments of any of the aspects, the level or activity of ZNF410 is decreased by at least 50%, at least 60%, at least 70%, at least 80%, at least 90%, or more as compared to an appropriate control. In some embodiments, a decreased level or activity of ZNF410, in a cell of the subject, increases the level and/or activity of fetal hemoglobin (HbF) by at least 50%, at least 60%, at least 70%, at least 80%, at least 90%, or more as compared to an appropriate control.

Synthetic Nucleic Acids and Gene Editing Compositions

In one aspect, the synthetic nucleic acid molecule provided herein comprises a nucleotide sequence selected from the group consisting of SEQ ID NO: 1-SEQ ID NO: 183 in Table 2, hybrids, truncations, or fragments thereof. The synthetic nucleic acid sequences are provided below.

TABLE 2
Synthetic nucleic acids targeting ZNF410 and their HbF enrichment scores.

| SEQ | | | HbF | | | | | | |
|---|---|---|---|---|---|---|---|---|---|
| ID | | AA | enrichment | Fitness | | | Exon | start | end |
| NO: | Synthetic nucleic acid sequence | position | score | score | AA | Exon | Length | coordinate | coordinate |
| 1 | ACCTTCTGCAGGACAGATGA | 249 | 1.8581 | −0.0768 | F | 7 | 181 | 74371618 | 74371638 |
| 2 | CACATCCTTGGGCTTCACAA | 310 | 1.8341 | −0.0101 | L | 8 | 89 | 74376059 | 74376079 |
| 3 | GGGAAGACCTTCTCTCAGAG | 349 | 1.7110 | 0.0823 | S | 9 | 125 | 74387711 | 74387731 |
| 4 | GAGCTGGCCATACAAATGTC | 228 | 1.6432 | 0.0463 | R | 6 | 150 | 74370765 | 74370785 |
| 5 | CTGGAAACCTGAAGAACCA | 297 | 1.6307 | −0.0063 | K | 7 | 181 | 74371750 | 74371770 |
| | C | | | | | | | | |
| 6 | GTTGTGACCGGACATTTGTA | 229 | 1.6201 | 0.1836 | T | 6 | 150 | 74370755 | 74370775 |
| 7 | AGAGGCTGAAGGTGCACAT | 268 | 1.6030 | −0.1102 | V | 7 | 181 | 74371663 | 74371683 |
| | G | | | | | | | | |
| 8 | GGCTTCAGTCACGGGAAGG | 41 | 1.5346 | 0.1084 | L | 3 | 135 | 74360587 | 74360607 |
| | A | | | | | | | | |
| 9 | AGGCTTCAGTCACGGGAAG | 41 | 1.5346 | 0.1084 | L | 3 | 135 | 74360588 | 74360608 |
| | G | | | | | | | | |
| 10 | CTGAGGCTTCAGTCACGGGA | 42 | 1.5346 | 0.1084 | L | 3 | 135 | 74360591 | 74360611 |
| 11 | CCAGACTCATGGCACATAA | 278 | 1.5088 | −0.0423 | P | 7 | 181 | 74371707 | 74371727 |
| | A | | | | | | | | |
| 12 | GCCAGACTCATGGCACATA | 279 | 1.5088 | −0.0423 | F | 7 | 181 | 74371708 | 74371728 |
| | A | | | | | | | | |
| 13 | CTCACCGATGAGTCTTGAGG | 239 | 1.5062 | −0.2295 | H | 6 | 150 | 74370798 | 74370818 |
| 14 | TTGCTCACCGATGAGTCTTG | 240 | 1.5062 | −0.2295 | L | 6 | 150 | 74370801 | 74370821 |
| 15 | AAACATCTGGTGGTTCACTC | 332 | 1.4467 | 0.1985 | V | 8 | 89 | 74376111 | 74376131 |
| 16 | TAGAATACTCAGCAAAGGA | 318 | 1.4290 | −0.0569 | R | 8 | 89 | 74376083 | 74376103 |
| | A | | | | | | | | |
| 17 | AATACCACCTCAAGACTCAT | 241 | 1.4028 | −0.3133 | K | 6 | 150 | 74370791 | 74370811 |
| 18 | CATTAGCCGACTGCATTCTG | 48 | 1.3921 | 0.0721 | S | 3 | 135 | 74360608 | 74360628 |
| 19 | GTACAGTTGAAGGTTGTGAC | 225 | 1.3879 | 0.0503 | G | 6 | 150 | 74370743 | 74370763 |
| 20 | CCACAGACTTGGCACTGATG | 338 | 1.3445 | −0.2010 | P | 9 | 125 | 74387693 | 74387713 |
| 21 | CACCTGTGTGGATGCGCCGG | 299 | 1.3360 | 0.1056 | H | 7 | 181 | 74371769 | 74371789 |
| 22 | ACACACCTGTGTGGATGCGC | 300 | 1.3360 | 0.1056 | R | 7 | 181 | 74371772 | 74371792 |
| 23 | TCCTGAACCCGGAGGAACC | 163 | 1.2525 | 0.0016 | P | 5 | 191 | 74364874 | 74364894 |
| | A | | | | | | | | |
| 24 | GCACAGACAGTAGCATTCC | 161 | 1.2300 | −0.0088 | S | 5 | 191 | 74364854 | 74364874 |
| | A | | | | | | | | |
| 25 | GGATGCGCCGGTGGTTCTTC | 296 | 1.2149 | −0.1190 | L | 7 | 181 | 74371760 | 74371780 |
| 26 | TCAGACTGTACAGAAATCCC | 96 | 1.2088 | −0.0524 | K | 4 | 218 | 74363121 | 74363141 |
| 27 | GCTACTGTCTGTGCTCTCAC | 155 | 1.1974 | −0.0504 | S | 5 | 191 | 74364848 | 74364868 |
| 28 | CCTTTCCTTTGTGAAGCCCA | 312 | 1.1798 | 0.1119 | E | 8 | 89 | 74376051 | 74376071 |
| 29 | AGCAGCTGGGAGTCAAGAG | 371 | 1.1765 | 0.2679 | Q | 9 | 125 | 74387776 | 74387796 |
| | C | | | | | | | | |
| 30 | CTGCAGGACAGATGAAGGA | 247 | 1.1649 | −0.1550 | R | 7 | 181 | 74371613 | 74371633 |
| | G | | | | | | | | |
| 31 | CTTTGTGAAGCCCAAGGATG | 314 | 1.1621 | −0.0745 | Q | 8 | 89 | 74376057 | 74376077 |
| 32 | AAAGGAACGGCCACATCCT | 314 | 1.1621 | −0.0745 | Q | 8 | 89 | 74376070 | 74376090 |
| | T | | | | | | | | |
| 33 | CAAAGGAACGGCCACATCC | 314 | 1.1621 | −0.0745 | Q | 8 | 89 | 74376071 | 74376091 |
| | T | | | | | | | | |
| 34 | TGAGTTAGAATCCAAACCA | 9 | 1.1339 | 0.0852 | K | 2 | 181 | 74358889 | 74358909 |
| | G | | | | | | | | |
| 35 | GACTGGGCTCACCTCTGGTT | 9 | 1.1339 | 0.0852 | K | 2 | 181 | 74358903 | 74358923 |
| 36 | AAGCTGACTGGGCTCACCTC | 10 | 1.1339 | 0.0852 | P | 2 | 181 | 74358908 | 74358928 |
| 37 | GAACCACCAGATGTTTTCGG | 326 | 1.1307 | −0.4464 | L | 8 | 89 | 74376107 | 74376127 |
| 38 | AGTGAACCACCAGATGTTTT | 327 | 1.1307 | −0.4464 | R | 8 | 89 | 74376110 | 74376130 |
| 39 | GTAGCATTCCATGGTTCCTC | 164 | 1.1272 | −0.1434 | W | 5 | 191 | 74364863 | 74364883 |
| 40 | TAGCATTCCATGGTTCCTCC | 165 | 1.1272 | −0.1434 | F | 5 | 191 | 74364864 | 74364884 |
| 41 | CCTGCTTCCACTCTGAGAGA | 348 | 1.1240 | 0.0317 | F | 9 | 125 | 74387721 | 74387741 |
| 42 | AAAGCTTCTATGTGCTGCAG | 262 | 1.1101 | 0.0216 | V | 7 | 181 | 74371645 | 74371665 |
| 43 | GGGCCAACTCCTGAACCCG | 166 | 1.0939 | −0.0189 | L | 5 | 191 | 74364882 | 74364902 |
| | G | | | | | | | | |
| 44 | TGAAGTGGACAAAAACTCC | 98 | 1.0828 | −0.0202 | P | 4 | 218 | 74363141 | 74363161 |
| | G | | | | | | | | |
| 45 | CTGAAGTGGACAAAAACTC | 98 | 1.0828 | −0.0202 | P | 4 | 218 | 74363142 | 74363162 |
| | C | | | | | | | | |
| 46 | TCTGAAGTGGACAAAAACT | 98 | 1.0828 | −0.0202 | P | 4 | 218 | 74363143 | 74363163 |
| | C | | | | | | | | |
| 47 | ATAAAAGAAGTGCTATCACT | 115 | 1.0758 | −0.1793 | P | 4 | 218 | 74363194 | 74363214 |
| 48 | CCTTGGGCTTCACAAAGGAA | 308 | 1.0723 | −0.0905 | P | 8 | 89 | 74376054 | 74376074 |
| 49 | TCTTTTCTCCCCCCAGCTCC | 11 | 1.0595 | 0.0078 | E | 2 | 181 | 74360483 | 74360503 |
| 50 | AACAAACTGTACCAGGAGC | 11 | 1.0595 | 0.0078 | E | 2 | 181 | 74360497 | 74360517 |
| | T | | | | | | | | |
| 51 | GAGGCTAGAATACTCAGCA | 320 | 1.0422 | −0.0407 | F | 8 | 89 | 74376088 | 74376108 |
| | A | | | | | | | | |
| 52 | GTGCACATGAGGACCCACA | 272 | 1.0284 | 0.0145 | T | 7 | 181 | 74371674 | 74371694 |
| | A | | | | | | | | |
| 53 | AAAGGGCTTCTCTCCATTGT | 273 | 1.0284 | 0.0145 | H | 7 | 181 | 74371690 | 74371710 |
| 54 | TAAAGGGCTTCTCTCCATTG | 273 | 1.0284 | 0.0145 | H | 7 | 181 | 74371691 | 74371711 |
| 55 | TCAGAATACGTCCATCCCAT | 22 | 1.0165 | 0.0076 | I | 3 | 135 | 74360516 | 74360536 |
| 56 | CAGAATACGTCCATCCCATT | 22 | 1.0165 | 0.0076 | | 3 | 135 | 74360517 | 74360537 |
| 57 | AAGCCCCTGTCCCAATGGGA | 22 | 1.0165 | 0.0076 | I | 3 | 135 | 74360530 | 74360550 |
| 58 | CTACAAGCCCCTGTCCCAAT | 23 | 1.0165 | 0.0076 | P | 3 | 135 | 74360534 | 74360554 |
| 59 | TCTACAAGCCCCTGTCCCAA | 23 | 1.0165 | 0.0076 | P | 3 | 135 | 74360535 | 74360555 |
| 60 | ATGTGCCATGAGTCTGGCTG | 284 | 1.0003 | −0.1170 | S | 7 | 181 | 74371710 | 74371730 |
| 61 | AACCACCGGCGCATCCACA | 302 | 0.9758 | −0.4036 | I | 7 | 181 | 74371764 | 74371784 |
| | C | | | | | | | | |
| 62 | TGGTGCTGCACACACCTGTG | 303 | 0.9758 | −0.4036 | H | 7 | 181 | 74371781 | 74371801 |
| 63 | AAGGCTTCTCAAAACAGCTG | 72 | 0.9607 | 0.0522 | S | 4 | 218 | 74363063 | 74363083 |
| 64 | AGCTGAGGAAGGGACCTCC | 67 | 0.9254 | 0.1188 | K | 4 | 218 | 74363048 | 74363068 |
| | T | | | | | | | | |
| 65 | TGCAGTCGGCTAATGTTACC | 54 | 0.9142 | −0.1413 | M | 3 | 135 | 74360613 | 74360633 |
| 66 | GTTCCTCCGGGTTCAGGAGT | 169 | 0.9027 | −0.0218 | Q | 5 | 191 | 74364876 | 74364896 |
| 67 | CATTCTGAGGCTTCAGTCAC | 43 | 0.8883 | −0.0669 | P | 3 | 135 | 74360595 | 74360615 |
| 68 | CCTTCTCTCAGAGTGGAAGC | 351 | 0.8767 | −0.0436 | S | 9 | 125 | 74387718 | 74387738 |
| 69 | CTGAAGCCTCAGAATGCAGT | 49 | 0.8482 | −0.0686 | E | 3 | 135 | 74360599 | 74360619 |
| 70 | AGAGCAGGAGCAAACTGGT | 376 | 0.8357 | −0.0505 | T | 9 | 125 | 74387791 | 74387811 |
| | G | | | | | | | | |
| 71 | TACGTCCATCCCATTGGGAC | 24 | 0.8191 | −0.0992 | L | 3 | 135 | 74360522 | 74360542 |
| 72 | ACGTCCATCCCATTGGGACA | 24 | 0.8191 | −0.0992 | L | 3 | 135 | 74360523 | 74360543 |
| 73 | CGTCCATCCCATTGGGACAG | 24 | 0.8191 | −0.0992 | L | 3 | 135 | 74360524 | 74360544 |
| 74 | ACAGGAGAAAATGTCCACC | 197 | 0.8011 | 0.0444 | V | 6 | 150 | 74370658 | 74370678 |
| | T | | | | | | | | |
| 75 | GCCCATCACCAGAACCAAG | 198 | 0.8011 | 0.0444 | H | 6 | 150 | 74370675 | 74370695 |
| | G | | | | | | | | |
| 76 | TGTCTGTGCTCTCACTGGAG | 153 | 0.7958 | 0.0570 | L | 5 | 191 | 74364843 | 74364863 |
| 77 | TGAGCACTTAGTGTTTGTAC | 140 | 0.7852 | −0.1409 | F | 5 | 191 | 74364789 | 74364809 |
| 78 | GAGAAAGCATCACCTGCAG | 363 | 0.7554 | 0.0551 | L | 9 | 125 | 74387752 | 74387772 |
| | C | | | | | | | | |
| 79 | AGAAAGCATCACCTGCAGC | 363 | 0.7554 | 0.0551 | L | 9 | 125 | 74387753 | 74387773 |
| | T | | | | | | | | |
| 80 | TCCCAGCTGCTCCCAGCTGC | 363 | 0.7554 | 0.0551 | L | 9 | 125 | 74387767 | 74387787 |
| 81 | AGTCAAGAGCAGGAGCAAA | 374 | 0.7541 | −0.5774 | E | 9 | 125 | 74387786 | 74387806 |
| | C | | | | | | | | |
| 82 | CAGCTGTTTTGAGAAGCCTT | 76 | 0.7462 | 0.0413 | R | 4 | 218 | 74363063 | 74363083 |
| 83 | AGCTGTTTTGAGAAGCCTTC | 77 | 0.7462 | 0.0413 | S | 4 | 218 | 74363064 | 74363084 |
| 84 | CCTCATCAGTGCCAAGTCTG | 342 | 0.7414 | 0.0826 | Q | 9 | 125 | 74387690 | 74387710 |
| 85 | CTCATCAGTGCCAAGTCTGT | 342 | 0.7414 | 0.0826 | Q | 9 | 125 | 74387691 | 74387711 |
| 86 | AGAAGGTCTTCCCACAGACT | 342 | 0.7414 | 0.0826 | Q | 9 | 125 | 74387704 | 74387724 |
| 87 | TTCAGTACCCAGTAAAAACC | 394 | 0.7021 | −0.2094 | K | 10 | 140 | 74388805 | 74388825 |
| 88 | TCCTTCATCTGTCCTGCAGA | 252 | 0.6953 | −0.0239 | P | 7 | 181 | 74371614 | 74371634 |
| 89 | CTTTTCCCACAACCTTCTGC | 252 | 0.6953 | −0.0239 | P | 7 | 181 | 74371629 | 74371649 |
| 90 | CTATGTGCTGCAGAGGCTGA | 265 | 0.6949 | −0.0599 | R | 7 | 181 | 74371652 | 74371672 |
| 91 | GAACAAACTGTACCAGGAG | 11 | 0.6673 | −0.2029 | E | 2 | 181 | 74360498 | 74360518 |
| | C | | | | | | | | |
| 92 | AAAGAATACCTGCTCTTGTT | 126 | 0.6666 | −0.1079 | L | 4 | 218 | 74363226 | 74363246 |
| 93 | TTCTAGCCTCCGAAAACATC | 328 | 0.6550 | −0.0193 | K | 8 | 89 | 74376098 | 74376118 |
| 94 | TAGCCTCCGAAAACATCTGG | 329 | 0.6550 | −0.0193 | H | 8 | 89 | 74376101 | 74376121 |
| 95 | ACCAAATACCAATTCTATCC | 417 | 0.6427 | −0.0316 | S | 10 | 140 | 74388874 | 74388894 |
| 96 | CCAAATACCAATTCTATCCT | 417 | 0.6427 | −0.0316 | S | 10 | 140 | 74388875 | 74388895 |
| 97 | CGGGTGAATGTGGGTCCAG | 83 | 0.6222 | 0.2364 | G | 4 | 218 | 74363083 | 74363103 |
| | A | | | | | | | | |
| 98 | GCTCTCGTCTCCTCTCCGTC | 84 | 0.6222 | 0.2364 | P | 4 | 218 | 74363101 | 74363121 |
| 99 | AGGTGGTATTTAAAGTGAGC | 233 | 0.6220 | 0.0665 | P | 6 | 150 | 74370781 | 74370801 |
| 100 | CAAGCTAGAAGACTCTGAA | 103 | 0.6053 | −0.0758 | T | 4 | 218 | 74363156 | 74363176 |
| | G | | | | | | | | |
| 101 | AATGCAAAAACCAGCAGCA | 191 | 0.6012 | 0.1332 | S | 5 | 191 | 74364943 | 74364963 |
| | A | | | | | | | | |
| 102 | ACGGGCCTCACCATTGCTGC | 191 | 0.6012 | 0.1332 | S | 5 | 191 | 74364956 | 74364976 |
| 103 | TTCTCAAAACAGCTGAGGA | 70 | 0.5806 | 0.2949 | P | 4 | 218 | 74363058 | 74363078 |
| | A | | | | | | | | |
| 104 | CTTCTCAAAACAGCTGAGGA | 70 | 0.5806 | 0.2949 | P | 4 | 218 | 74363059 | 74363079 |
| 105 | AGCAGCAATCAAACTGTCAT | 173 | 0.5781 | −0.0565 | H | 5 | 191 | 74364902 | 74364922 |
| 106 | TAGCAGCAATCAAACTGTCA | 173 | 0.5781 | −0.0565 | H | 5 | 191 | 74364903 | 74364923 |
| 107 | CTTCTTAACCTAACAAGAGC | 127 | 0.5745 | 0.0476 | T | 4 | 218 | 74363215 | 74363235 |
| 108 | TCCATGGTTCCTCCGGGTTC | 167 | 0.5648 | −0.1896 | R | 5 | 191 | 74364870 | 74364890 |
| 109 | CATGGGCCAACTCCTGAACC | 167 | 0.5648 | −0.1896 | R | 5 | 191 | 74364885 | 74364905 |
| 110 | TATTCTGAACAAACTGTACC | 13 | 0.5528 | 0.1666 | L | 3 | 135 | 74360504 | 74360524 |
| 111 | AAGGAGATTTAACTGAAAG | 474 | 0.5325 | 0.1210 | T | 12 | 1008 | 74398215 | 74398235 |
| | A | | | | | | | | |
| 112 | AAATCATTCTAACTCCTCCA | 65 | 0.5301 | 0.0600 | S | 4 | 218 | 74363028 | 74363048 |
| 113 | TCATTCTAACTCCTCCAAGG | 66 | 0.5301 | 0.0600 | S | 4 | 218 | 74363031 | 74363051 |
| 114 | TGAGGAAGGGACCTCCTTG | 66 | 0.5301 | 0.0600 | S | 4 | 218 | 74363045 | 74363065 |
| | G | | | | | | | | |
| 115 | GGATAGAATTGGTATTTGGT | 412 | 0.5066 | −0.0114 | L | 10 | 140 | 74388874 | 74388894 |
| 116 | CCCAGGATAGAATTGGTATT | 413 | 0.5066 | −0.0114 | P | 10 | 140 | 74388878 | 74388898 |
| 117 | GCATTCTGAGGCTTCAGTCA | 44 | 0.4992 | 0.0386 | V | 3 | 135 | 74360596 | 74360616 |
| 118 | CACATCAGGCACTGAAGAC | 434 | 0.4873 | 0.0866 | L | 11 | 127 | 74390127 | 74390147 |
| | A | | | | | | | | |
| 119 | TCACATCAGGCACTGAAGA | 434 | 0.4873 | 0.0866 | L | 11 | 127 | 74390128 | 74390148 |
| | C | | | | | | | | |
| 120 | CACTGAAGACAGGGAACGT | 431 | 0.4847 | −0.0516 | P | 11 | 127 | 74390118 | 74390138 |
| | G | | | | | | | | |
| 121 | GCACTGAAGACAGGGAACG | 431 | 0.4847 | −0.0516 | P | 11 | 127 | 74390119 | 74390139 |
| | T | | | | | | | | |
| 122 | GGCACTGAAGACAGGGAAC | 431 | 0.4847 | −0.0516 | P | 11 | 127 | 74390120 | 74390140 |
| | G | | | | | | | | |
| 123 | CAGGATGAGGCAGAAGATT | 146 | 0.4782 | 0.0202 | E | 5 | 191 | 74364808 | 74364828 |
| | C | | | | | | | | |
| 124 | AGGATGAGGCAGAAGATTC | 146 | 0.4782 | 0.0202 | E | 5 | 191 | 74364809 | 74364829 |
| | A | | | | | | | | |
| 125 | CTGTGCAGCTGAGCCACTAA | 379 | 0.4540 | −0.0664 | P | 10 | 140 | 74388760 | 74388780 |
| 126 | TGTGCAGCTGAGCCACTAAT | 379 | 0.4540 | −0.0664 | P | 10 | 140 | 74388761 | 74388781 |
| 127 | AGCAAACTACTGCCCATTAG | 379 | 0.4540 | −0.0664 | P | 10 | 140 | 74388776 | 74388796 |
| 128 | GGTTCAAGGACTCTCCACCA | 405 | 0.4369 | 0.0038 | L | 10 | 140 | 74388853 | 74388873 |
| 129 | AAAAACCAGCAGCAATGGT | 193 | 0.4193 | −0.2509 | N | 5 | 191 | 74364948 | 74364968 |
| | G | | | | | | | | |
| 130 | TCTTACCATCATCAACTCCC | 419 | 0.4099 | −0.1303 | L | 10 | 140 | 74388895 | 74388915 |
| 131 | TTCTGGGCCCCTTCCTCAAG | 213 | 0.3487 | −0.2038 | P | 6 | 150 | 74370705 | 74370725 |
| 132 | TTGAGCTTCTTTTCCACTTG | 213 | 0.3487 | −0.2038 | P | 6 | 150 | 74370721 | 74370741 |
| 133 | ATCTGTCCTGCAGAAGGTTG | 254 | 0.2929 | −0.0006 | E | 7 | 181 | 74371620 | 74371640 |
| 134 | TCTGTCCTGCAGAAGGTTGT | 254 | 0.2929 | −0.0006 | E | 7 | 181 | 74371621 | 74371641 |
| 135 | CTTTTACAGTTACTAAACCA | 468 | 0.2927 | 0.0398 | L | 12 | 1008 | 74398196 | 74398216 |
| 136 | GTCCACCTTGGTTCTGGTGA | 201 | 0.2905 | 0.0369 | S | 6 | 150 | 74370670 | 74370690 |
| 137 | TCCACCTTGGTTCTGGTGAT | 201 | 0.2905 | 0.0369 | S | 6 | 150 | 74370671 | 74370691 |
| 138 | AACTGCAGTAAATCCACAA | 464 | 0.2764 | 0.0513 | P | 11 | 127 | 74390203 | 74390223 |
| | G | | | | | | | | |
| 139 | AGAGACCACTTTACCTCTTG | 464 | 0.2764 | 0.0513 | P | 11 | 127 | 74390219 | 74390239 |
| 140 | CTCTCCACCAAGGCTGGGCT | 402 | 0.2120 | 0.0302 | Q | 10 | 140 | 74388843 | 74388863 |
| 141 | ACTCTCCACCAAGGCTGGGC | 402 | 0.2120 | 0.0302 | Q | 10 | 140 | 74388844 | 74388864 |
| 142 | CTGAAAGACGGACATGAGC | 478 | 0.2073 | −0.2308 | T | 12 | 1008 | 74398227 | 74398247 |
| | G | | | | | | | | |
| 143 | ATTTTGTTTCTTCCAGGTCT | 129 | 0.2067 | 0.1009 | A | 4 | 218 | 74364757 | 74364777 |
| 144 | TCAGCTGAAGAGCCCAGAC | 129 | 0.2067 | 0.1009 | A | 4 | 218 | 74364772 | 74364792 |
| | C | | | | | | | | |
| 145 | GCTGGGAATTCATAGACACC | 396 | 0.2036 | −0.1399 | L | 10 | 140 | 74388826 | 74388846 |
| 146 | GGGCAGTAGTTTGCTTGAAG | 386 | 0.2015 | −0.1397 | L | 10 | 140 | 74388781 | 74388801 |
| 147 | CCCTTTATGTGCCATGAGTC | 282 | 0.1998 | −0.0078 | H | 7 | 181 | 74371704 | 74371724 |
| 148 | GCTTACCACAGCCAGACTCA | 282 | 0.1998 | −0.0078 | H | 7 | 181 | 74371718 | 74371738 |
| 149 | CTGGACCCACATTCACCCGA | 78 | 0.1993 | 0.0609 | L | 4 | 218 | 74363082 | 74363102 |
| 150 | GAATGTGGGTCCAGACGGA | 85 | 0.1567 | −0.1139 | D | 4 | 218 | 74363088 | 74363108 |
| | G | | | | | | | | |
| 151 | CTTAGTGTTTGTACAGGATG | 142 | 0.1533 | 0.2595 | Q | 5 | 191 | 74364795 | 74364815 |
| 152 | GGTAAGCAGTTTACTACAGC | 291 | 0.1483 | 0.3114 | T | 7 | 181 | 74371731 | 74371751 |
| 153 | GAAAATGTCCACCTTGGTTC | 199 | 0.1438 | −0.0186 | L | 6 | 150 | 74370664 | 74370684 |
| 154 | ACTGCCCATCACCAGAACCA | 199 | 0.1438 | −0.0186 | L | 6 | 150 | 74370678 | 74370698 |
| 155 | GTCTTTCAGTTAAATCTCCT | 470 | 0.1438 | 0.0735 | Q | 12 | 1008 | 74398216 | 74398236 |
| 156 | ACCAGGTGATGTGTCACATC | 438 | 0.1324 | 0.0773 | P | 11 | 127 | 74390141 | 74390161 |
| 157 | TGCTGCTACTCGTGCACAAC | 182 | 0.1312 | −0.0161 | A | 5 | 191 | 74364915 | 74364935 |
| 158 | ATAGACACCAGGTTTTTACT | 392 | 0.0951 | 0.0228 | P | 10 | 140 | 74388815 | 74388835 |
| 159 | CATAGACACCAGGTTTTTAC | 393 | 0.0951 | 0.0228 | S | 10 | 140 | 74388816 | 74388836 |
| 160 | ATTGGTATTTGGTAGGTTCA | 410 | 0.0784 | −0.0345 | L | 10 | 140 | 74388867 | 74388887 |
| 161 | ATGAATTCCCAGCCCAGCCT | 403 | 0.0482 | 0.0417 | P | 10 | 140 | 74388833 | 74388853 |
| 162 | AAGGACTCTCCACCAAGGCT | 403 | 0.0482 | 0.0417 | P | 10 | 140 | 74388848 | 74388868 |
| 163 | AATTCCCAGCCCAGCCTTGG | 404 | 0.0482 | 0.0417 | S | 10 | 140 | 74388836 | 74388856 |
| 164 | CAAGGACTCTCCACCAAGG | 404 | 0.0482 | 0.0417 | S | 10 | 140 | 74388849 | 74388869 |
| | C | | | | | | | | |
| 165 | GATGGGCAGTCAAAAGATT | 207 | 0.0456 | 0.0591 | K | 6 | 150 | 74370688 | 74370708 |
| | C | | | | | | | | |
| 166 | ATGGGCAGTCAAAAGATTCT | 207 | 0.0456 | 0.0591 | K | 6 | 150 | 74370689 | 74370709 |
| 167 | GCCTGATGTGACACATCACC | 442 | 0.0313 | −0.0527 | H | 11 | 127 | 74390137 | 74390157 |
| 168 | GAGAAGCCTTCGGGTGAAT | 80 | 0.0240 | 0.2185 | V | 4 | 218 | 74363073 | 74363093 |
| | G | | | | | | | | |
| 169 | AGAAGCCTTCGGGTGAATGT | 80 | 0.0240 | 0.2185 | V | 4 | 218 | 74363074 | 74363094 |
| 170 | CACCTGCAGCTGGGAGCAG | 366 | −0.0202 | 0.0016 | G | 9 | 125 | 74387762 | 74387782 |
| | C | | | | | | | | |
| 171 | ACCTGCAGCTGGGAGCAGC | 366 | −0.0202 | 0.0016 | G | 9 | 1251 | 74387763 | 74387783 |
| | T | | | | | | | | |
| 172 | CACCTGGTGACCATGCAGTC | 447 | −0.0704 | 0.1304 | 2 | 11 | 127 | 74390153 | 74390173 |
| 173 | ACCTGGTGACCATGCAGTCA | 447 | −0.0704 | 0.1304 | M | 11 | 127 | 74390154 | 74390174 |
| 174 | TGATTGCCTCCCTGACTGCA | 447 | −0.0704 | 0.1304 | M | 11 | 127 | 74390166 | 74390186 |
| 175 | TGGTGACCATGCAGTCAGG | 448 | −0.0704 | 0.1304 | Q | 11 | 127 | 74390157 | 74390177 |
| | G | | | | | | | | |
| 176 | TTCTTTTCCACTTGAGGAAG | 211 | −0.0860 | 0.0876 | P | 6 | 150 | 74370715 | 74370735 |
| 177 | CTTCTTTTCCACTTGAGGAA | 212 | −0.0860 | 0.0876 | L | 6 | 150 | 74370716 | 74370736 |
| 178 | GCTTCTTTTCCACTTGAGGA | 212 | −0.0860 | 0.0876 | L | 6 | 150 | 74370717 | 74370737 |
| 179 | ATCAACTCCCAGGATAGAAT | 416 | −0.1495 | −0.0245 | N | 10 | 140 | 74388885 | 74388905 |
| 180 | TCCCTGACTGCATGGTCACC | 444 | −0.1945 | 0.2563 | L | 11 | 127 | 74390158 | 74390178 |
| 181 | AAGCTCAAGTGTACAGTTGA | 222 | −0.3227 | 0.0831 | T | 6 | 150 | 74370733 | 74370753 |
| 182 | TTCACAGAGGTGCTTGCTGA | 426 | −0.3689 | 0.0587 | L | 11 | 127 | 74390090 | 74390110 |
| 183 | TCTATCCTGGGAGTTGATGA | 421 | −0.4860 | 0.1743 | V | 10 | 140 | 74388887 | 74388907 |

*starting and ending coordinates are compared to a reference sequence HG19 genome for chromosome 14 ZNF410 Ensemble Gene ID: ENSG00000119725

In some embodiments of any of the aspects, the synthetic nucleic acid molecule nucleotide sequence is selected from the group consisting of: SEQ ID NO: 1-SEQ ID NO: 169, and fragments thereof. In some embodiments of any of the aspects, the synthetic nucleic acid molecule comprises a nucleotide sequence GTACAGTTGAAGGTTGTGAC (SEQ ID NO: 19) or a fragment thereof. In some embodiments, the synthetic nucleic acid provided herein targets sequences between exons 6-9 encoding the cluster of five C2H2 zinc fingers of ZNF410.

In some embodiments of any of the aspects, the synthetic nucleic acid molecule polynucleotide sequence is at least 15 nucleotides in length or more, 16 nucleotides in length or more, 17 nucleotides in length or more, 18 nucleotides in length or more, 19 nucleotides in length or more, 20 nucleotides in length or more, 21 nucleotides in length or more, 22 nucleotides in length or more, 23 nucleotides in length or more, 24 nucleotides in length or more, up to 25 nucleotides in length.

In some embodiments, the synthetic nucleic acid molecule is a DNA strand, RNA strand, or a hybrid thereof. For example, the nucleic acid can be an RNA, wherein the uracil (U) nucleobases are modified or replaced with thymine (T) nucleobases. Examples of such modifications are discussed herein below or known in the art.

Exemplary nucleic acid modifications include, but are not limited to, nucleobase modifications, sugar modifications, inter-sugar linkage modifications, conjugates (e.g., ligands), and combinations thereof. Nucleic acid modifications are known in the art, see, e.g., US20160367702A1; US20190060458A11; U.S. Pat. Nos. 8,710,200; and 7,423,142, which are incorporated herein by reference in their entireties.

Exemplary modified nucleobases include, but are not limited to, thymine (T), inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

Exemplary sugar modifications include, but are not limited to, 2′-Fluoro, 3′-Fluoro, 2′-OMe, 3′-OMe, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH2-C(═O)—N(H)-5′) and amide-4 (3′-CH2-N(H)—C(═O)-5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH2-O-5′), formacetal (3′-O—CH2-O-5′), oxime, methyleneimino, methykenecarbonylamino, methylenemethylimino (MMI, 3′-CH2-N(CH3)-O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH2-S—C5′, C3′-O—P(O)—O—SS—C5′, C3′-CH2—NH—NH—C5′, 3′-NHP(O)(OCH3)-O-5′ and 3′-NHP(O)(OCH3)-O-5′

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or other xeno nucleic acids (XNA) described in the art.

In some embodiments of any of the aspects, the synthetic nucleic acid molecule provided herein is a guide RNA (gRNA). A guide RNA is a nucleic acid molecule that targets and hybridizes to a target sequence of a DNA molecule. As used herein, “hybridizes” or “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The sequence of the guide RNA (e.g., the sequence homologous to the target gene of interest) can be determined for the intended use. For example, to target ZNF410, in a one would choose a guide RNA that targets and hybridize to ZNF410 in a manner that effectively results in the desired alteration of the gene's expression. In general, a guide sequence is any polynucleotide sequence having sufficient complementarity with a target polynucleotide sequence to hybridize with the target sequence and direct sequence-specific binding of a nuclease provided herein (e.g., a CRISPR nuclease enzyme).

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a nuclease and/or CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything) has a single occurrence in the genome. A unique target sequence in a genome may include an S. pyogenes Cas9 target site of the form MMMMMMMMNNNNNNNNNNNNXGG where NNNNNNNNNNNNXGG (N is A, G, T, or C; and X can be anything) has a single occurrence in the genome.

In some embodiments of any of the aspects, the synthetic nucleic acid molecule further comprises a scaffold nucleic acid. In some embodiments of any of the aspects, the scaffold nucleic acid comprises the sequence

(SEQ ID NO: 184)
GUUUUAGAGCUAGAAAUAGCAAGUUAAAAUAAGGCUAGUCCGUUAUCAAC
UUGAAAAAGUGGCACCGAGUCGGUGCUUUU

In some embodiments of any of the aspects, the synthetic nucleic acid molecule further comprises a crNRA, a tracrRNA, or a hybrid sequence thereof.

In one aspect, provided herein is a gene editing composition comprising: a synthetic nucleic acid molecule selected from the group consisting of those listed in Table 2; and a nuclease.

In some embodiments of any of the aspect, the nuclease is selected from the group consisting of: a Cas enzyme, a CRISPR enzyme, a ZFN, a TALEN, a meganuclease, a base editing nuclease, an a prime editing nuclease.

Genome editing cannot be performed using traditional restriction endonucleases since most restriction enzymes recognize a few base pairs on the DNA as their target and the probability is very high that the recognized base pair combination will be found in many locations across the genome resulting in multiple cuts (i.e., not limited to a desired location). To overcome this challenge and create site-specific double-stranded breaks, several distinct classes of nucleases have been discovered and bioengineered to date. These are the meganucleases, Zinc finger nucleases (ZFNs), Cas9/CRISPR system, and transcription-activator like effector nucleases (TALENs).

Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001), Nucleic Acids Res. 29(18): 3757-3774). The LAGLIDADG meganucleases with a single copy of the LAGLIDADG motif form homodimers, whereas members with two copies of the LAGLIDADG are found as monomers. Similarly, the GIY-YIG family members have a GIY-YIG module, which is 70-100 residues long and includes four or five conserved sequence motifs with four invariant residues, two of which are required for activity (see Van Roey et al. (2002), Nature Struct. Biol. 9: 806-811). The His-Cys box meganucleases are characterized by a highly conserved series of histidines and cysteines over a region encompassing several hundred amino acid residues (see Chevalier et al. (2001), Nucleic Acids Res. 29(18): 3757-3774). In the case of the NHN family, the members are defined by motifs containing two pairs of conserved histidines surrounded by asparagine residues (see Chevalier et al. (2001), Nucleic Acids Res. 29(18): 3757-3774). The four families of meganucleases are widely separated from one another with respect to conserved structural elements and, consequently, DNA recognition sequence specificity and catalytic activity.

Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision BioSciences' Directed Nuclease Editor™ genome editing technology.

ZFNs and TALENs restriction endonuclease technology utilizes a non-specific DNA cutting enzyme which is linked to a specific DNA sequence recognizing peptide(s) such as zinc fingers and transcription activator-like effectors (TALEs). Typically, an endonuclease whose DNA recognition site and cleaving site are separate from each other is selected and the its cleaving portion is separated and then linked to a sequence recognizing peptide, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Although the nuclease portions of both ZFNs and TALEs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALENs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs for use with the methods and compositions described herein can be obtained commercially from e.g., Sangamo Biosciences™ (Richmond, Calif.).

In some embodiments of any of the aspects, the gene editing composition is a CRISPR system. Clustered regularly interspaced short palindromic repeats (CRISPR)/CRISPR-associated (Cas) systems is useful for RNA-programmable genome editing (see e.g., Jinek, M. et al. Science (2012) 337(6096):816-821).

In some embodiments of any of the aspects, the Cas enzyme is selected from the group consisting of: S. pyogenes Cas9 (spCas9), Cas9 (also known as Csn1 and Csx12), Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cash, Cas7, Cas8, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

A CRISPR system refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the methods and compositions provided herein, an exogenous template polynucleotide may be referred to as an editing template. In an aspect of the methods and compositions provided herein, the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a cell such that expression of the elements of the CRISPR system direct formation of a CRISPR complex at one or more target sites.

Methods of Preparing Synthetic Nucleic Acids and Gene Editing Compositions

The synthetic nucleic acids and gene editing compositions provided herein can be prepared by synthetic methods known in the art including, but not limited to, chemical synthesis, including but not limited to a nucleoside phosphoramidite approach, or in vitro transcription among others. Methods for chemical synthesis to include modified nucleotides are also known in the art.

In in vitro transcription, polymerases can be used including, but not limited to, bacteriophage polymerase such as T7 polymerase, T3 polymerase and SP6 polymerase, viral polymerases, and E. coli RNA polymerase.

Oligonucleotide strands can be isolated from a sample using RNA extraction and purification methods know in the art. These methods include but are not limited to column purification, ethanol precipitation, phenol-chloroform extraction, or acid guanidinium thiocyanate-phenol chloroform extraction (AGPC).

For therapeutic purposes, the synthetic nucleic acids and gene editing compositions provided herein should have a degree of stability in serum to allow distribution and cellular uptake. The prolonged maintenance of therapeutic levels of the oligonucleotides in serum will have a significant effect on the distribution and cellular uptake and unlike conjugate groups that target specific cellular receptors, the increased serum stability will affect all cells.

Chemical modifications can also include the addition of ligands, linkers, and peptides. For example, the ligand can improve stability, hybridization thermodynamics with a target nucleic acid, targeting to a particular tissue or cell-type, or cell permeability, e.g., by an endocytosis-dependent or independent mechanism. Oligonucleotides bearing peptide conjugates can be prepared using procedures known in the art.

The gene editing compositions provided herein comprise a nuclease enzyme that can be isolated, purified, and/or engineered by any method known in the art. In some embodiments, the nuclease enzyme is a DNA-targeting endonuclease provided herein.

Methods of preparing gene editing compositions and nucleases for gene editing applications are described, e.g., in Takeuchi et al. Method Mol Biol. 1239: 105-132, (2015); Carroll D. Genome engineering with zinc-finger nucleases. Genetics. 2011; 188:773-782; Bogdanove A J, Voytas D F. TAL effectors: customizable proteins for DNA targeting. Science. 2011; 333:1843-1846.; Gao H, Smith J, Yang M, Jones S, Djukanovic V, Nicholson M G, West A, Bidney D, Falco S C, Jantz D, et al. Heritable targeted mutagenesis in maize using a designed endonuclease. The Plant journal: for cell and molecular biology. 2010; 61:176-187; Zheng Y, Roberts R J. Selection of restriction endonucleases using artificial cells. Nucleic acids research. 2007; 35:e83.; Daboussi F, Zaslayskiy M, Poirot L, Loperfido M, Gouble A, Guyot V, Leduc S, Galetto R, Grizot S, Oficjalska D, et al. Chromosomal context and epigenetic mechanisms control the efficacy of genome editing by rare-cutting designer endonucleases. Nucleic acids research. 2012; 40:6367-6379; WO 2019/213273, WO2019/147302, U.S. Pat. No. 9,982,279B1; US20180282762A1; US20150031133A1; U.S. Pat. No. 9,404,098B2; WO2014022702A2; U.S. Pat. No. 8,697,359B1; U.S. Ser. No. 10/428,319B2; and U.S. Pat. No. 9,790,490B2 which are incorporated herein by reference in their entireties. Furthermore, nuclease enzymes are commercially available, e.g., MISSION™ Cas9 Proteins (Millipore Sigma, St. Louis, Mo.).

Agents

The methods provided herein comprise agents that decrease the levels or activity of ZNF410.

In some embodiments of any of the aspects, the agent is selected from the group consisting of: a nucleic acid; a gene editing composition; a small molecule; a dominant negative ZNF410 polypeptide; and an antibody.

In some embodiments, the agent that decreases the levels or activity of ZNF410 is a dominant negative ZNF410 polypeptide. Dominant negative polypeptides are reviewed, e.g., in Sheppard D. Dominant negative mutants: tools for the study of protein function in vitro and in vivo. Am J Respir Cell Mol Biol. 1994; 11(1):1-6, which is incorporated herein by reference in its entirety.

In some embodiments, the gene editing composition comprises: (a) at least one synthetic nucleic acid molecule selected from the group consisting of Table 2; and (b) a nuclease. Non-limiting examples of nuclease enzymes are provided above.

In one aspect, provided herein is a nanoparticle that reduces the levels or activity of ZNF410. In some embodiments, the nanoparticle comprises an agent, synthetic nucleic acid molecule, or gene editing composition provided herein.

In another aspect, provided herein is a vector comprising the agent, synthetic nucleic acid molecule, or gene editing composition provided herein. In some embodiments the vector is a viral vector.

In another aspect, provided herein is a cell that has been contacted with the gene editing composition, vector, or nanoparticle provided herein. In another aspect, provided herein is a method of transplanting a cell that has been contacted with the gene editing composition provided herein. It is contemplated herein that hematopoietic stem cells can be edited to reduce the levels of ZNF410 and increase the expression of HbF. Subsequently, the edited cells can be xenotransplanted into the subject by methods known in the art.

Pharmaceutical Compositions

The methods and compositions provided herein can further comprise formulating the synthetic nucleic acids, agents, gene editing compositions, vectors, and nanoparticles provided herein with a pharmaceutically acceptable carrier.

Administration of the synthetic nucleic acids, agents, and gene editing compositions provided herein can include formulation into pharmaceutical compositions or pharmaceutical formulations for parenteral administration, e.g., intravenous; mucosal, or other mode of administration. In some embodiments, the synthetic nucleic acids, agents, and gene editing compositions provided herein can be administered along with any pharmaceutically acceptable carrier compound, material, or composition which results in an effective treatment in the subject.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, synthetic nucleic acids, agents, and gene editing compositions provided herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

The agents and compositions provided herein can be formulated for administration to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) transdermally; (3) transmucosally; (4) via bronchoalveolar lavage.

In some embodiments of any of the aspects, the agents and compositions provided herein comprise a particle or polymer-based vehicle. Exemplary particle or polymer-based vehicles include, but are not limited to, nanoparticles, microparticles, polymer microspheres, or polymer-drug conjugates.

In some embodiments of any of the aspects, the agents and compositions provided herein further comprise a lipid vehicle. Exemplary lipid vehicles include, but are not limited to, liposomes, phospholipids, micelles, lipid emulsions, and lipid-drug complexes.

In some embodiments of any of the aspects, the agents and compositions provided herein are formulated in a composition comprising micelles, amphiphilic carriers, polymers, cyclodextrins, liposomes, and encapsulation devices.

Microemulsification technology can improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In some embodiments of any of the aspects, the agents and compositions provided herein can be formulated with an amphiphilic carrier. Amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

The agents and compositions as described herein can be formulated with hydrophilic polymers. Hydrophilic polymers are water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Other hydrophilic polymers which may be suitable include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a pharmaceutical composition as described herein comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In certain embodiments, an agents or pharmaceutical composition described herein is formulated as a liposome. Liposomes can be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic D D, Liposomes from physics to applications, Elsevier Science Publishers BV, Amsterdam, 1993. The active ingredients of the pharmaceutical compositions described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Administration, Dosage, Efficacy

The synthetic nucleic acid molecules, agents, gene editing compositions, vectors, nanoparticles, and pharmaceutical compositions provided herein can be formulated, dosed, and administered in a fashion consistent with good medical practice. Factors for consideration in this context include the particular disorder being treated, the particular subject being treated, the clinical condition of the individual subject, the cause of the disorder, the site of delivery of the composition, the method of administration, the scheduling of administration, and other factors known to medical practitioners.

The therapeutic formulations to be used for in vivo administration, such as parenteral administration, in the methods described herein can be sterile, which is readily accomplished by filtration through sterile filtration membranes, or other methods known to those of skill in the art.

The agents and pharmaceutical compositions thereof as provided herein can be administered to a subject in need thereof by any appropriate route which results in an effective treatment in the subject. As used herein, the terms “administering,” and “introducing” are used interchangeably and refer to the placement of a composition into a subject by a method or route which results in at least partial localization of such compositions at a desired site, such that a desired effect(s) is produced. A pharmaceutical composition can be administered to a subject by any mode of administration that delivers the agent, synthetic nucleic acid, or gene editing composition systemically or to a desired surface or target, and can include, but is not limited to, injection, infusion, instillation, and inhalation administration. To the extent that the agent/composition can be protected from inactivation in the gut, oral administration forms are also contemplated. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a therapeutic agent other than directly into a target site, tissue, or organ, such as a tumor site, such that it enters the subject's circulatory system and, thus, is subject to metabolism and other like processes.

The term “effective amount” as used herein refers to the amount of a composition needed to alleviate or prevent at least one or more symptom of a hemoglobinopathy, disease or disorder, relates to a sufficient amount of pharmacological composition to provide the desired effect, e.g., increase fetal hemoglobin levels (HbF), reduce pathology, or any symptom associated with or caused by the disease. The term “therapeutically effective amount” therefore refers to an amount of an agent or composition described herein using the methods as disclosed herein, that is sufficient to result in a particular effect when administered to a typical subject. An effective amount as used herein would also include an amount sufficient to delay the development of a symptom of the disease, alter the course of a symptom disease (for example, but not limited to, slow the progression of a symptom of the disease), or reverse a symptom of the disease. Thus, it is not possible to specify the exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the antigen or fragment thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The agents or gene editing compositions described herein can be formulated, in some embodiments, with one or more additional therapeutic agents currently used to prevent or treat hemoglobinopathy, for example, hydroxyurea. The effective amount of such other agents depend on the amount of the agent/compositions provided herein in the formulation, the type of disorder or treatment, and other factors discussed above. These are generally used in the same dosages and with administration routes as used herein before or about from 1 to 99% of the heretofore employed dosages.

The dosage ranges for the agents or pharmaceutical compositions provide herein depend upon the potency, and encompass amounts large enough to produce the desired effect. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 μg/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. These doses can be administered by one or more separate administrations, or by continuous infusion. For repeated administrations over several days or longer, depending on the condition, the treatment is sustained until, for example, the hemoglobinopathy is treated, as measured by the methods described above or known in the art.

However, other dosage regimens can be useful. The duration of a therapy using the methods described herein will continue for as long as medically indicated or until a desired therapeutic effect (e.g., those described herein) is achieved. In certain embodiments, the administration of the pharmaceutical composition described herein is continued for 1 month, 2 months, 4 months, 6 months, 8 months, 10 months, 1 year, 2 years, 3 years, 4 years, 5 years, 10 years, 20 years, or for a period of years up to the lifetime of the subject.

As will be appreciated by one of skill in the art, appropriate dosing regimens for a given composition can comprise a single administration or multiple ones. Subsequent doses may be given repeatedly at time periods, for example, about two weeks or greater up through the entirety of a subject's life, e.g., to provide a sustained preventative effect. The precise dose to be employed in the formulation will also depend on the route of administration and should be decided according to the judgment of the practitioner and each patient's circumstances. Ultimately, the practitioner or physician will decide the amount of the agent or composition thereof to administer to particular subjects.

“Alleviating a symptom of a hemoglobinopathy” is ameliorating any condition or symptom associated with the a hemoglobinopathy as provided herein. Alternatively, alleviating a symptom of a hemoglobinopathy can involve increasing the fetal hemoglobin levels in the subject relative to such load in an untreated control. The levels of fetal hemoglobin can be measured by any standard technique known in the art.

A patient who is being treated for a hemoglobinopathy is one who a medical practitioner has diagnosed as having such a condition. Diagnosis may be by any suitable means. Diagnosis and monitoring may involve, for example, detecting the level of microbial load in a biological sample (for example, a tissue biopsy, blood test, or urine test), detecting the level of a surrogate marker of the disease in a biological sample, detecting symptoms associated with a hemoglobinopathy provided herein. One of skill in the art will understand that these patients may have been subjected to the same standard tests as described above or may have been identified, without examination, as one at high risk due to the presence of one or more risk factors (such as family history).

Exemplary embodiments of the various aspects described herein can be described by one or more of the following numbered embodiments:

Embodiment 1: A synthetic nucleic acid molecule capable of targeting zinc finger 410 (ZNF410), the nucleic acid molecule comprising: a nucleotide sequence selected from the group consisting of SEQ ID NO: 1-SEQ ID NO: 183 in Table 2.

Embodiment 2: The synthetic nucleic acid molecule of Embodiment 1, wherein the nucleic acid molecule is a guide RNA (gRNA).

Embodiment 3: The synthetic nucleic acid molecule of any one of Embodiments 1-2, further comprising a scaffold nucleic acid.

Embodiment 4: The synthetic nucleic acid molecule of any one of Embodiments 1-2, further comprising a crRNA, a tracrRNA, or a hybrid sequence thereof.

Embodiment 5: The synthetic nucleic acid molecule of any one of Embodiments 1-4, wherein the synthetic nucleic acid molecule is at least 15 nucleotides in length.

Embodiment 6: The synthetic nucleic acid molecule of Embodiment 3, wherein the scaffold nucleic acid comprises the sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 184).

Embodiment 7: A gene editing composition comprising: (a)(a) a synthetic nucleic acid molecule selected from the group consisting of Table 2; and (b) a nuclease enzyme.

Embodiment 8: The gene editing composition of Embodiment 7, wherein the synthetic nucleic acid molecule nucleotide sequence is selected from the group consisting of: SEQ ID NO: 1-SEQ ID NO: 169.

Embodiment 9: The gene editing composition of Embodiment 7 or Embodiment 8, wherein the synthetic nucleic acid molecule comprises a nucleotide sequence GTACAGTTGAAGGTTGTGAC (SEQ ID NO: 19).

Embodiment 10: The gene editing composition of any one of Embodiments 7-9, wherein the nuclease enzyme is selected from the group consisting of: a Cas enzyme, a CRISPR enzyme, a ZFN, a TALEN, a meganuclease, a base editing nuclease, and a prime editing nuclease.

Embodiment 11: The gene editing composition of Embodiment 10, wherein the Cas enzyme is selected from the group consisting of: S. pyogenes Cas9 (spCas9), Cas9 (also known as Csn1 and Csx12), Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cash, Cas7, Cas8, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

Embodiment 12: The gene editing composition of any one of Embodiments 7-11, wherein the gene editing composition is capable of targeting zinc finger 410 (ZNF410).

Embodiment 13: A vector comprising the synthetic nucleic acid molecule of any of Embodiments 1-6; or the gene editing composition of any one of Embodiments 7-12.

Embodiment 14: The vector of Embodiment 13, wherein the vector is a viral vector.

Embodiment 15: The vector of Embodiment 14, wherein the viral vector is selected from the group consisting: a lentiviral vector; an adeno-associated viral vector (AAV); a recombinant adeno-associated viral vector (rAAV); a retroviral vector; a foamyviral vector; and a alpharetroviral vector.

Embodiment 16: A nanoparticle comprising the synthetic nucleic acid molecule of any of Embodiments 1-6; or the gene editing composition of any one of Embodiments 7-12.

Embodiment 17: A pharmaceutical composition comprising: (a) the gene editing composition of any one of Embodiments 7-12; or (b) the vector of any one of Embodiments 13-15; or (c) the nanoparticle of Embodiment 16; and (d) a pharmaceutically acceptable carrier.

Embodiment 18: A method of increasing fetal hemoglobin level in a subject, the method comprising: administering to a subject: (a) the gene editing composition of any one of Embodiments 7-12; or (b) the vector of any one of Embodiments 13-15; or (c) the nanoparticle of Embodiment 16; or (d) the pharmaceutical composition of Embodiment 17.

Embodiment 19: A method of treating a hemoglobinopathy in a subject, the method comprising: administering to a subject in need thereof an agent that decreases the level or activity of ZNF410.

Embodiment 20: A method of increasing fetal hemoglobin level in a subject, the method comprising: administering to a subject an agent that decreases the level or activity of ZNF410.

Embodiment 21: The method of any one of Embodiments 19-20, wherein the agent is selected from the group consisting of: a nucleic acid; a gene editing composition; a small molecule; a dominant negative ZNF410 polypeptide; and an antibody.

Embodiment 22: The method of Embodiment 21, wherein the gene editing composition comprises: (a) at least one synthetic nucleic acid molecule selected from the group consisting of Table 2; and (b) a nuclease.

Embodiment 23: The method of any one of Embodiments 19-22, wherein the hemoglobinopathy is a β-hemoglobinopathy.

Embodiment 24: The method of any one of Embodiments 19-23, wherein the hemoglobinopathy is selected from the group consisting of: sickle cell disease; sickle cell anemia; sickle-hemoglobin C disease (HbSC); sickle beta-plus-thalassemia (HbS/β+); sickle beta-zero-thalassemia (HbS/β0); and β-thalassemia.

Embodiment 25: The method of any one of Embodiments 19-24, wherein the level or activity of ZNF410 is decreased by at least 50%, at least 60%, at least 70%, at least 80%, at least 90%, or more as compared to an appropriate control.

Embodiment 26: The method of any one of Embodiments 19-25, wherein a decreased level or activity of ZNF410, in a cell of the subject, increases the level and/or activity of fetal hemoglobin (HbF) by at least 50%, at least 60%, at least 70%, at least 80%, at least 90%, or more as compared to an appropriate control.

Embodiment 27: The method of any one of Embodiments 19-26, wherein the subject is a mammal.

Embodiment 28: The method of any one of Embodiments 19-27, wherein the subject is a human.

Additional exemplary embodiments of the various aspects described herein can be described by one or more of the following embodiments:

Embodiment 1: A method of increasing fetal hemoglobin level in a subject, the method comprising: administering to a subject in need thereof an agent that decreases the level or activity of zinc finger 410 (ZNF410).

Embodiment 2: The method of Embodiment 1, wherein the subject has a hemoglobinopathy.

Embodiment 3: A method of treating a hemoglobinopathy in a subject, the method comprising: administering to a subject in need thereof an agent that decreases the level or activity of ZNF410.

Embodiment 4: The method of any one of Embodiments 1-3, wherein the agent is a nucleic acid comprising a nucleotide sequence complementary to at least a portion of a nucleic acid encoding ZNF410, an anti-ZNF410 antibody, a zinc finger inhibitor, or a dominant negative ZNF410 polypeptide.

Embodiment 5: The method of Embodiment 4, wherein the nucleic acid agent is a guide RNA, a sgRNA, an siRNA, a shRNA, an antisense oligonucleotide, an aptamer, a ribozyme, a DNAzyme, or a microRNA.

Embodiment 6: The method of any one of Embodiments 1-5, wherein the agent is a nucleic acid comprising a nucleotide sequence selected from SEQ ID NO: 1-SEQ ID NO: 183.

Embodiment 7: The method of Embodiment 6, wherein the agent is a nucleic acid comprising a nucleotide sequence selected from SEQ ID NO: 1-SEQ ID NO: 169.

Embodiment 8: The method of Embodiment 7, wherein the agent is a nucleic acid comprising a nucleotide sequence GTACAGTTGAAGGTTGTGAC (SEQ ID NO: 19).

Embodiment 9: The method of any one of Embodiments 1-5, wherein the agent is an anti-ZNF410 antibody selected from anti-ZNF410 antibody HPA002871 (Sigma-Aldrich), anti-ZNF410 antibody SAB2104187 (Sigma-Aldrich), anti-ZNF410 antibody SAB1407818 (Sigma-Aldrich), anti-ZNF410 antibody NBP2-21008 (Novus Biologicals), anti-ZNF410 antibody ABIN2777798 (antibodies-online.com), anti-ZNF410 antibody ABIN931225 (antibodies-online.com), anti-ZNF410 antibody ABIN1882025 (antibodies-online.com), anti-ZNF410 antibody ABIN2155007 (antibodies-online.com), anti-ZNF410 antibody ABIN5535398 (antibodies-online.com), anti-ZNF410 antibody ABIN6742349 (antibodies-online.com), anti-ZNF410 antibody ABIN528331 (antibodies-online.com), anti-ZNF410 antibody ABIN2687242 (antibodies-online.com), anti-ZNF410 antibody ABIN5516407 (antibodies-online.com), ZNF410 Polyclonal antibody (Invitrogen), ZNF410 antibody N1C1 (GeneTex), ZNF410 antibody MBS8501808 (MyBioSource.Com), ZNF410 antibody MBS9125687 (MyBioSource.Com), Proteintech #14529-1-AP and RRID:AB 2257520.

Embodiment 10: The method of any one of Embodiments 1-5, wherein the zinc finger inhibitor is a zinc finger ejector compound.

Embodiment 11: The method of Embodiment 10, wherein the zinc finger ejector compound is selected from the group consisting of azodicarbonamide (ADA), 3-nitrosobenzamide (NOBA), 6-nitroso-1,2-benzopyrone (NOBP), 2,2′-di-thiobisbenzamide (DIBA), mercaptobenzamides, Pyridinioalkanoyl thiolesters (PATES), and bis-thiadizolbenzene-1,2-diamine.

Embodiment 12: The method of any one of Embodiments 2-11, wherein the hemoglobinopathy is a β-hemoglobinopathy.

Embodiment 13: 1The method of Embodiment 12, wherein the hemoglobinopathy is selected from the group consisting of: sickle cell disease; sickle cell anemia; sickle-hemoglobin C disease (HbSC); sickle beta-plus-thalassemia (HbS/β+); sickle beta-zero-thalassemia (HbS/β0); and β-thalassemia.

Embodiment 14: The method of any one of Embodiments 1-13, wherein the subject is a mammal.

Embodiment 15: The method of Embodiment 14, wherein the subject is human.

Embodiment 16: A synthetic nucleic acid molecule comprising a nucleotide sequence selected from the group consisting of SEQ ID NO: 1-SEQ ID NO: 183.

Embodiment 17: The synthetic nucleic acid molecule of Embodiment 16, wherein the nucleic acid molecule is single-stranded.

Embodiment 18: The synthetic nucleic acid molecule of Embodiment 16, wherein the nucleic acid molecule is double-stranded or partially double-stranded.

Embodiment 19: The synthetic nucleic acid molecule of Embodiment 16, wherein the nucleic acid molecule comprises a hairpin.

Embodiment 20: The synthetic nucleic acid molecule of any one of Embodiments 16-19, wherein the nucleic acid molecule comprises a nucleotide sequence selected from the group consisting of SEQ ID NO: 1-SEQ ID NO: 169.

Embodiment 21: The synthetic nucleic acid molecule of any one of Embodiments 16-20, wherein the nucleic acid molecule comprises the nucleotide sequence

(SEQ ID NO: 19)
GTACAGTTGAAGGTTGTGAC

Embodiment 22: The synthetic nucleic acid molecule of any one of Embodiments 16-21, wherein the nucleic acid molecule is an RNA molecule.

Embodiment 23: The synthetic nucleic acid molecule of any one of Embodiments 16-22, wherein the nucleic acid molecule is a guide RNA (gRNA), an siRNA, a shRNA, an antisense oligonucleotide, an aptamer, a ribozyme or a microRNA.

Embodiment 24: The synthetic nucleic acid molecule of any one of Embodiments 16-23, wherein the nucleic acid molecule is a guide RNA (gRNA) and further comprises a scaffold nucleic acid sequence.

Embodiment 24: The synthetic nucleic acid molecule of Embodiment 24, wherein the scaffold nucleic acid sequence comprises the nucleotide sequence:

(SEQ ID NO: 184)
GUUUUAGAGCUAGAAAUAGCAAGUUAAAAUAAGGCUAGUCCGUUAUCAAC
UUGAAAAAGUGGCACCGAGUCGGUGCUUUU

Embodiment 26: The synthetic nucleic acid molecule of any one of Embodiments 16-25, wherein the nucleic acid molecule further comprising a crRNA, a tracrRNA, or a hybrid sequence thereof.

Embodiment 27: The synthetic nucleic acid molecule of any one of Embodiments 16-26, wherein the synthetic nucleic acid molecule is at least 21 nucleotides in length.

Embodiment 28: A composition comprising a synthetic nucleic acid molecule of any one of Embodiments 16-27.

Embodiment 29: The composition of Embodiment 28, wherein the composition further comprises a nuclease enzyme.

Embodiment 30: The composition of Embodiment 29, wherein the nuclease enzyme is selected from the group consisting of: a Cas enzyme, a CRISPR enzyme, a ZFN, a TALEN, a meganuclease, a base editing nuclease, an argonaute protein, and a prime editing nuclease.

Embodiment 31: The composition of Embodiment 30, wherein the Cas enzyme is selected from the group consisting of: S. pyogenes Cas9 (spCas9), Cas9 (also known as Csn1 and Csx12), Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cash, Cas7, Cas8, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

Embodiment 32: The composition of any one of Embodiments 28-31, wherein the composition is in the form of a particle.

Embodiments 33: The composition of Embodiment 32, wherein the particle is a nanoparticle.

Embodiment 34: The composition of any one of Embodiments 28-31, wherein the composition is a pharmaceutical composition and further comprises a pharmaceutically acceptable excipient or carrier.

Embodiment 35: A vector comprising the synthetic nucleic acid molecule of any of Embodiments 16-27.

Embodiment 36: The vector of Embodiment 35, wherein the vector further comprises a polynucleotide comprising a nucleotide sequence encoding a nuclease enzyme.

Embodiment 37: The vector of Embodiment 36, wherein the nuclease enzyme is selected from the group consisting of: a Cas enzyme, a CRISPR enzyme, a ZFN, a TALEN, a meganuclease, a base editing nuclease, an argonaute protein, and a prime editing nuclease.

Embodiment 38: The vector of any one of Embodiments 35-37, wherein the vector is a viral vector.

Embodiment 39: The vector of Embodiment 38, wherein the viral vector is selected from the group consisting: a lentiviral vector; an adeno-associated viral vector (AAV); a recombinant adeno-associated viral vector (rAAV); a retroviral vector; a foamyviral vector; and a alpharetroviral vector.

Embodiment 40: A composition comprising a vector of any one of Embodiments 35-39.

Embodiment 41: The composition of Embodiment 40, wherein the composition is in the form of a particle.

Embodiment 42: The composition of Embodiment 41, wherein the particle is a nanoparticle.

Embodiment 43: The composition of any one of Embodiments 40-43, wherein the composition is a pharmaceutical composition and further comprises a pharmaceutically acceptable excipient or carrier.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, an “agent” can be any chemical, entity or moiety, including without limitation synthetic and naturally-occurring proteinaceous and non-proteinaceous entities. In some embodiments, an agent is a nucleic acid, nucleic acid analog, protein, antibody, peptide, aptamer, oligomer of nucleic acids, amino acids, or carbohydrates including without limitation a protein, oligonucleotide, ribozyme, DNAzyme, glycoprotein, guideRNA, sgRNA, siRNAs, lipoprotein, nanoparticle, and/or a modification or combinations thereof etc. In certain embodiments, agents are small molecule chemical moieties. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

An agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertions and other variants.

As used herein, “small molecules” include, but are not limited to, small peptides or peptide-like molecules, soluble peptides, and non-peptidyl organic or inorganic compounds. For example, a small molecule inhibitor of ZNF410 can have a molecular weight of about 100 to about 20,000 Daltons (Da), for example about 500 to about 15,000 Da, or about 1000 to about 10,000 Da.

A “nucleic acid”, as described herein, can be RNA or DNA, and can be single or double stranded, and can be selected, for example, from a group including: nucleic acid encoding a protein of interest, oligonucleotides, nucleic acid analogues, for example, peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to guideRNA, single guide RNA, RNAi, shRNAi, siRNA, micro RNAi (mRNAi), antisense oligonucleotides, etc.

As used herein, the term “RNA” refers to ribonucleic acid, which as typically transcribed in nature comprises the purine nucleobases adenine and guanine and the pyrimidine nucleobases cytosine and uracil. RNA oligonucleotides described herein can include modified nucleobases or modifications to the ribose-phosphate backbone that, for example, enhance stability or resistance to degradation.

As used herein, the terms “antibody” or “antibodies” or “antigen-binding fragments thereof” include monoclonal, human, humanized or chimeric antibodies, single chain antibodies, Fab fragments, F(ab′) fragments, fragments produced by a Fab expression library, and/or antigen-binding fragments of any of the above. Antibodies can also refer to immunoglobulin molecules and immunologically active portions that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art. In some embodiments, the antibody provided herein targets zinc finger 410 (ZNF410) polypeptide.

By “targets zinc finger 410 (ZNF410)” is meant that the nucleic acid molecules, agents, and/or gene editing compositions provided herein interact, contact, or bind directly to a ZNF410 polypeptide or nucleic acid encoding the ZNF410 polypeptide. In some embodiments, the agents, synthetic nucleic acid molecules, and/or gene editing composition provided herein decrease the level of expression of ZNF410. At a minimum, the targeting of ZNF410 can be assayed by determining the amount of ZNF410 binding to the ZNF410 binding partner (e.g., an agent or synthetic nucleic acid molecule provided herein) using techniques standard in the art, including, but not limited to, mass spectrometry, immunoprecipitation, or gel filtration assays. ZNF410 activity can be assayed by measuring fetal hemoglobin expression at the mRNA or protein level following treatment with a candidate agent (e.g., a small molecule, nucleic acid, or polypeptide).

As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. In the present specification, “plasmid” and “vector” can be used interchangeably as the plasmid is the most commonly used form of vector. However, the methods and compositions described herein can include such other forms of expression vectors, such as viral vectors (e.g., replication defective retroviruses, lentiviruses, adenoviruses and adeno-associated viruses), which serve equivalent functions.

As used herein, the term “viral vector” refers to a nucleic acid vector construct that includes at least one element of viral origin and has the capacity to be packaged into a viral particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

As used herein, the terms “gene editing” or “genome editing” are used interchangeably to refer to a reverse genetics method and compositions thereof that use artificially engineered nucleases to cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point.

In one embodiment, as used herein, the term “DNA-targeting endonuclease” refers to an endonuclease that generates a double-stranded break at a desired position in the genome without producing undesired off-target double-stranded breaks. The DNA targeting endonuclease can be a naturally occurring endonuclease (e.g., a bacterial meganuclease) or it can be artificially generated (e.g., engineered meganucleases, TALENs, or ZFNs, among others).

In another embodiment, as used herein, the term “DNA-targeting endonuclease” refers to an endonuclease that generates a single-stranded break or a “nick” or break on one strand of the DNA phosphate sugar backbone at a desired position in the genome without producing undesired off-target DNA stranded breaks.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. In some embodiments, the polypeptide provided herein is a dominant-negative ZNF410 polypeptide.

The term “therapeutically effective amount” refers to an amount of an agent, synthetic nucleic acid molecule, or a gene editing system as provided herein, that is effective to treat a disease or disorder (e.g., a hemoglobinopathy) as the terms “treat” or “treatment” are defined herein. Amounts will vary depending on the specific disease or disorder, its state of progression, age, weight and gender of a subject, among other variables. Thus, it is not possible to specify an exact “effective amount”. However, for any given case, an appropriate “effective amount” can be determined by one of ordinary skill in the art using only routine experimentation.

As used herein, the term “administering,” refers to the placement of an agent, synthetic nucleic acid molecule, or pharmaceutical composition provided herein into a subject by a method or route which results in at least partial delivery of the agent or pharmaceutical compositions provided herein at a desired site.

As used herein, the term “treating” or “treatment” includes reducing or alleviating at least one adverse effect or symptom of a condition, disease or disorder. For example, the term “treating” and “treatment” refers to administering to a subject an effective amount of a composition, e.g., an effective amount of a composition comprising a population of hematopoietic progenitor cells so that the subject has a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment can improve the disease condition, but may not be a complete cure for the disease. In some embodiments, treatment can include prophylaxis. However, in alternative embodiments, treatment does not include prophylaxis.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

As used herein, the terms “prevention” or “preventing,” when used in reference to a disease, disorder or symptoms thereof, refers to a reduction in the likelihood that an individual will develop a disease or disorder, e.g., a hemoglobinopathy. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

As used herein, a “subject” is a human, mammal, or a non-human animal. Usually the non-human animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases including diseases and disorders involving inappropriate immunosuppression. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of treatment for a particular condition can be a subject having that condition, diagnosed as having that condition, or at risk of developing that condition (e.g., a hemoglobinopathy).

Accordingly, in one embodiment, the subject or the mammal has been diagnosed with a hemoglobinopathy. In a further embodiment, the hemoglobinopathy is a β-hemoglobinopathy. In one preferred embodiment, the hemoglobinopathy is a sickle cell disease. As used herein, “sickle cell disease” can be sickle cell anemia, sickle-hemoglobin C disease (HbSC), sickle beta-plus-thalassaemia (HbS/β+), or sickle beta-zero-thalassaemia (HbS/β0). In another embodiment, the hemoglobinopathy is a β-thalassemia.

As used herein, the term “hemoglobinopathy” means any defect in the structure or function of any hemoglobin of an individual, and includes defects in the primary, secondary, tertiary or quaternary structure of hemoglobin caused by any mutation, such as deletion mutations or substitution mutations in the coding regions of the β-globin gene, or mutations in, or deletions of, the promoters or enhancers of such genes that cause a reduction in the amount of hemoglobin produced as compared to a normal or standard condition. The term further includes any decrease in the amount or effectiveness of hemoglobin, whether normal or abnormal, caused by external factors such as disease, chemotherapy, toxins, poisons, or the like.

As used herein, the terms “synthetic” and “engineered” and their grammatical equivalents as used herein can refer to one or more human-designed alterations of a nucleic acid or gene editing composition provided herein.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given synthetic nucleic acid molecule, agent, or composition described herein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the invention, yet open to the inclusion of unspecified elements, whether essential or not.

As used herein, the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin Exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

It is to be understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### ZNF410 Represses Fetal Globin by Devoted Control of CHD4/NuRD

Major regulators of adult-stage fetal globin silencing have been identified, including the transcription factors (TFs) BCL11A, ZBTB7A/LRF, and the NuRD chromatin complex, although each has potential on-target liabilities for rational β-hemoglobinopathy therapeutic targeting. Here through CRISPR screening, ZNF410 was discovered to be a novel fetal hemoglobin (HbF) repressing TF that is dispensable for erythroid maturation. In contrast to BCL11A and ZBTB7A, ZNF410 does not bind directly to the -globin genes. Its predominant site of chromatin occupancy is CHD4, encoding the NuRD nucleosome remodeler, itself required for HbF repression. CHD4 has two ZNF410-bound regulatory elements with 27 combined ZNF410 binding motifs constituting unparalleled genomic clusters. These elements completely account for ZNF410's effects on -globin repression. Knockout of ZNF410 reduces CHD4 by 60%, enough to substantially de-repress HbF while avoiding the cellular toxicity of complete CHD4 loss. Mice with constitutive deficiency of the homolog Zfp410 are born at expected Mendelian ratios with unremarkable hematology. ZNF410 is unnecessary for human hematopoietic engraftment potential and erythroid maturation unlike known HbF repressors. These studies identify a new rational target for HbF induction for the fl-hemoglobin disorders with a wide therapeutic index. More broadly, ZNF410 represents a special class of gene regulator, a conserved transcription factor wholly devoted to exquisite regulation of a chromatin subcomplex.

### Introduction

Despite renewed enthusiasm for novel approaches to β-hemoglobinopathies, the clinical unmet need for these most common monogenic diseases remains vast¹⁻³. Induction of fetal-globin gene expression could bypass the underlying-globin molecular defects and ameliorate the pathophysiological cascades that result in elevated morbidity and mortality. Critical regulators of the switch from fetal to adult globin gene expression include the DNA-binding transcription factors (TFs) BCL11A and ZBTB7A and the nucleosome remodeling and deacetylase (NuRD) chromatin complex4-6. BCL11A and ZBTB7A each bind to unique sites at the proximal promoters of the duplicated fetal-globin genes HBG1 and HBG2 and each physically interact with NuRD5,7-9. Although the molecular details underpinning this switch, including the precise sequences bound and NuRD subcomplex members required, are increasingly understood, still the feasibility to pharmacologically directly perturb these mechanisms remains uncertain. One challenge is the pleiotropic molecular, cellular and organismal effects of each of the aforementioned HbF repressors which makes the therapeutic window uncertain and the risk of undesired on-target liabilities considerable. An ideal therapeutic target would have a wide therapeutic window through which inhibition of function could be tolerated across a diverse set of cellular contexts.

To better define additional molecular players orchestrating the developmental regulation of globin gene expression, a CRISPR screen focusing on putative DNA-binding TFs that contribute to HbF silencing was performed. ZNF410 was identified herein as a novel DNA-binding TF required for HbF repression. Little was previously known about ZNF410. It is demonstrated that indeed this gene is required for HbF silencing. Surprisingly, it displays a narrowly restricted pattern of chromatin occupancy, not binding to the globin locus directly, but rather binding through an unusual set of clustered motifs to upstream elements controlling the expression of the catalytic NuRD subunit CHD4. Finally, ZNF410, or its mouse homolog Zfp410, are dispensable for survival to adulthood as well as normal erythropoiesis and hematopoietic repopulation.

Results

CRISPR Screen for Novel Transcriptional Regulators of HbF Level

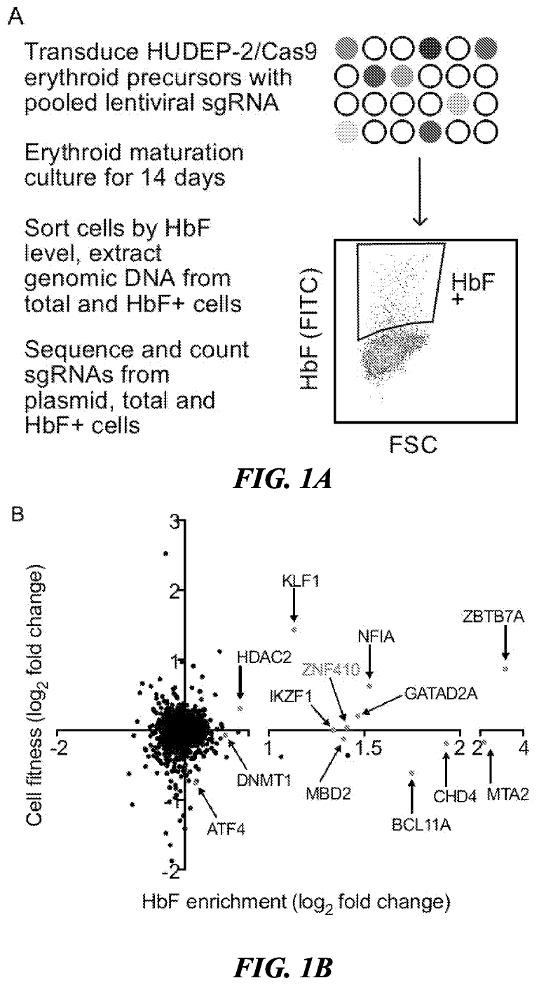

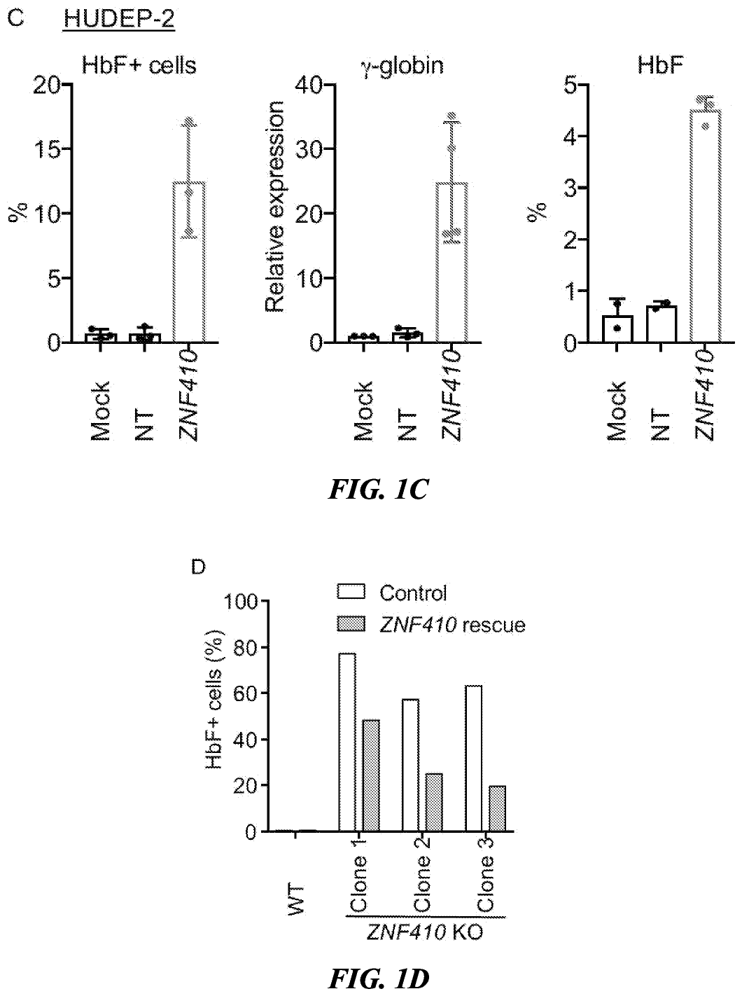

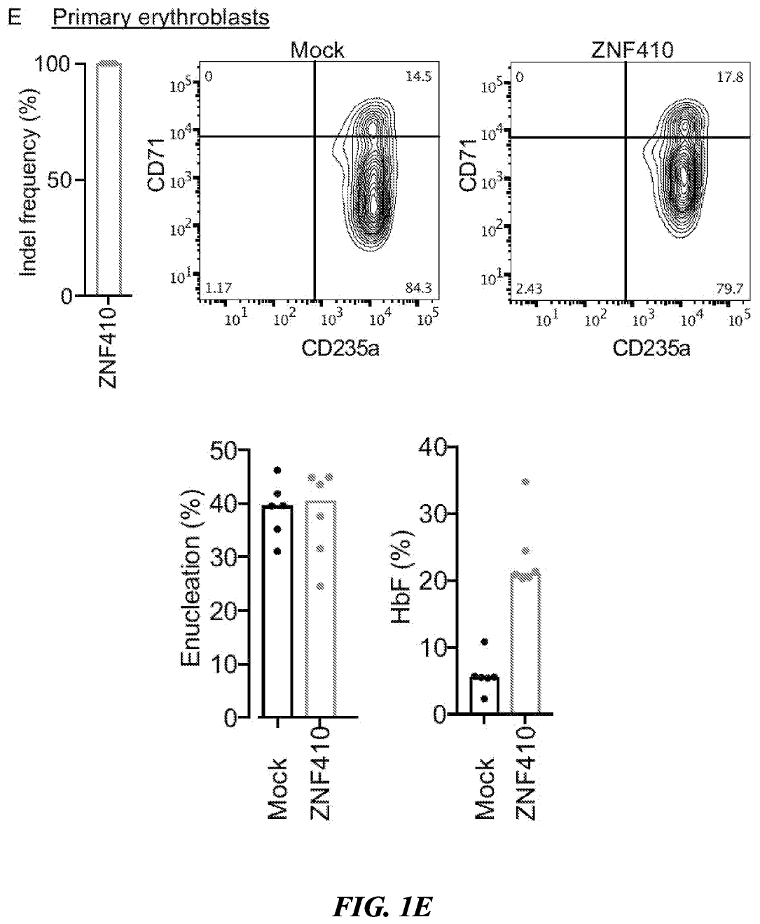

A CRISPR screen was performed in a primary human erythroid precursor cell line (HUDEP-2) that expresses an adult-type pattern of globins to discover genes required for repression of fetal hemoglobin. The screen included 19,092 sgRNA targeting 1591 transcription factors and 13 genes of the NuRD complex as controls. A stable line of HUDEP-2 cells expressing SpCas9 was generated. HUDEP-2/Cas9 cells were transduced by the sgRNA library at low multiplicity and selected for sgRNA cassette integration by acquisition of puromycin resistance. Following erythroid maturation culture, cells were stained for HbF expression and HbF+ cells (range 1.87%) acquired by FACS (FIG. 1A). Integrated sgRNAs were amplified from genomic DNA and counted by deep sequencing. Two sgRNA enrichment scores were calculated. First, sgRNA abundance was compared in HbF-high and total cells at the end of erythroid maturation to obtain an HbF enrichment score. Second, sgRNA abundance was compared in cells at the end of erythroid maturation and the starting library to define a fitness score. Negative fitness scores indicate relative depletion whereas positive fitness scores indicate relative enrichment of cells bearing these sgRNA compared to the overall library.

As expected, it was found that known HbF regulators like BCL11A and ZBTB7A showed highly elevated HbF enrichment scores (FIG. 1B). For BCL11A a negative fitness score was observed suggesting that loss of this gene had a modest negative impact on cell accumulation in vitro. For ZBTB7A positive fitness scores were demonstrated, suggesting cells mutated at this gene accumulated in the population. Consistent with this, mutagenesis of ZBTB7A produced delayed erythroid maturation suggesting impairment of terminal erythroid maturation as has been previously described. In addition, previous findings that a NuRD sub-complex comprised of CHD4, MTA2, GATAD2A, MBD2, and HDAC2 was required for HbF control were validated. As previously observed, editing CHD4 led to potent HbF induction but was associated with negative cell fitness.

The sgRNAs targeting ZNF410 were associated with robust HbF induction (FIG. 1B, Table 2). Unlike other regulators like BCL11A, ZBTB7A, and CHD4, no fitness effects of targeting ZNF410 were found. Little is known about ZNF410. ZNF410 is a zinc finger protein with a cluster of five C2H2 zinc fingers. ZNF410 has not been previously implicated in globin gene regulation or erythropoiesis. One previous report by Benanti, et al. in 2002 indicated that over-expression of ZNF410 in human foreskin fibroblasts (HFFs) led to increased expression of matrix remodeling genes MMP1, PAI2 and MMP12¹⁰. ZNF410 sumoylation was associated with extended protein half-life. The biochemical functions and biological roles of endogenous ZNF410 remain largely unexplored. Therefore, the focus was on ZNF410 as a novel regulator of HbF.

Validation in HUDEP-2 Cells and Primary Adult Erythroid Precursors

First, the role of ZNF410 in HbF repression was evaluated by targeting it with individual gRNAs. Upon editing in a bulk population of cells, induction of HbF was determined, as measured by HbF+ cells by intracellular flow cytometry, HBG1/2 expression by RT-qPCR, and HbF induction by HPLC (FIG. 1C). Three single cell derived HUDEP-2 ZNF410 deficient clones were generated. The unique biallelic KO clones were evaluated by genotyping. In each clone, the fraction of HbF+ cells were elevated. Upon re-expression of ZNF410, HbF was partially silenced, consistent with a causal role of ZNF410 in repressing HbF (FIG. 1D).

Next, the role of ZNF410 in HbF repression was evaluated in primary erythroblasts derived from erythroid culture of adult mobilized peripheral blood CD34+ HSPCs. Using cells from 3 independent donors, it was found that targeting ZNF410 by 3×NLS-SpCas9:sgRNA RNP electroporation gave robust indels to 100%, with all of these indels resulting in a +1 insertion and thus a frameshift allele (FIG. 1E). ZNF410 targeted erythroblasts displayed normal erythroid maturation and enucleation, similar to controls, and had robust induction of HbF to a median of 21.1% across samples (FIG. 1E). The effect of ZNF410 editing to that of editing the known HbF repressor TFs BCL11A and ZBTB7A alone and in combination were compared. Editing ZNF410 resulted in 100% frameshift edits and edits were preserved at the end of culture, on day 18, at

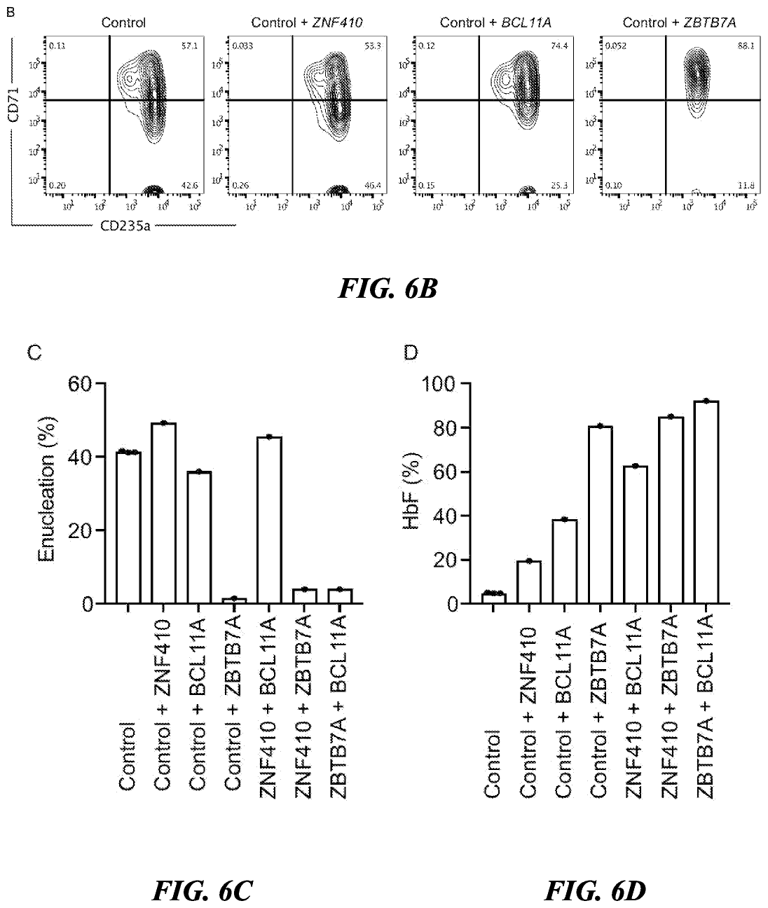

similar levels as on day 4 (FIG. 6A). Editing BCL11A resulted in 100% overall edits with 94% frameshifts on day 4 of culture. While overall edits were largely preserved, there was a decrease in frameshift alleles to 78% at the end of culture. Editing ZBTB7A resulted in 92% overall edits with 88% frameshifts on day 4 of culture. There was an increase in overall ZBTB7A edits to 100% with 94% frameshifts at the end of culture, consistent with positive fitness scores in the screen (FIG. 1B). Following BCL11A editing there was a modest decrease in the fraction of cells with the CD7110R′CD235a+ mature immunophenotype (from 42.6 to 25.3%) and a modest decrease in the fraction of enucleated cells (from 41.2 to 36%) (FIG. 6B, 6C). HbF was increased from 4.8% in controls to 38% after BCL11A editing (FIG. 6D). Combined editing of ZNF410 and BCL11A showed immunophenotype and enucleation fraction similar to controls, with HbF level of 63% (FIG. 6B-6D). These results suggest an additive relationship between ZNF410 and BCL11A in HbF repression. ZBTB7A editing led to a severe decrease in the fraction of cells with the CD71^{low}CD235a+ mature immunophenotype (from 42.6 to 11.8%) and nearly complete block in the production of enucleated cells (from 41.2% to 1.5%) (FIG. 6B-6C). HbF was increased from 4.8% in controls to 81% after ZBTB7A editing (FIG. 6D). Combined editing of ZNF410 and ZBTB7A showed immunophenotype and enucleation fraction similar to that of ZBTB7A single edited cells, with dramatic HbF induction and maturation block (FIG. 6B-D). Together these results show ZNF410 is a novel HbF repressor that appears to be dispensable for erythropoiesis.

ZNF410 is a Special DNA-Binding Protein

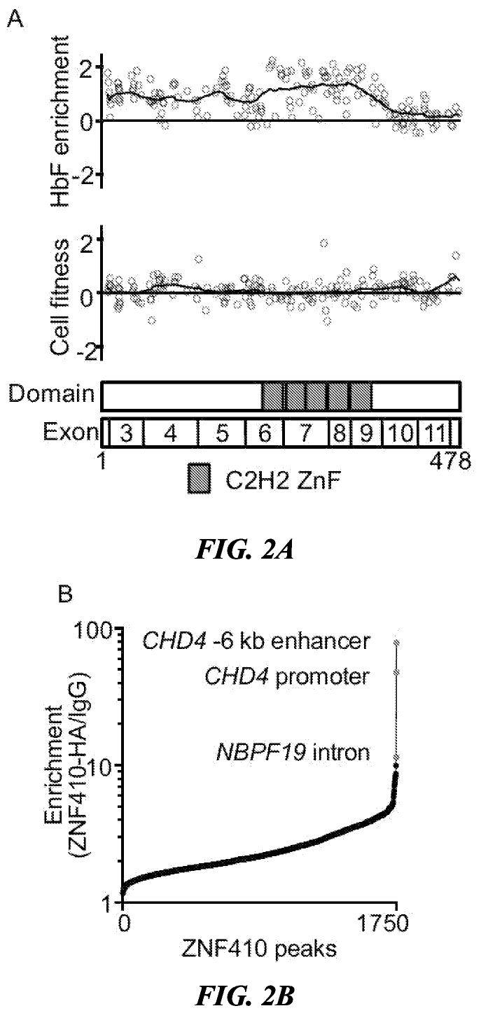

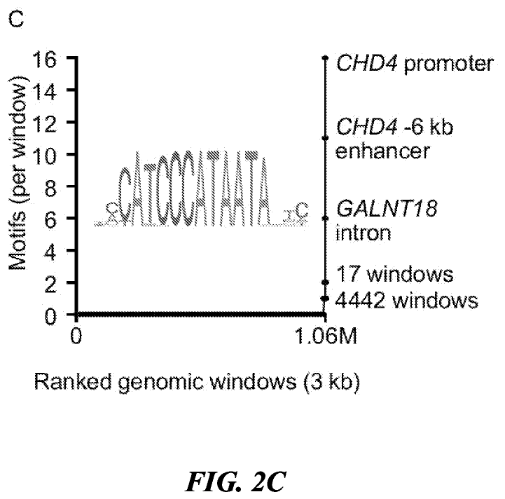

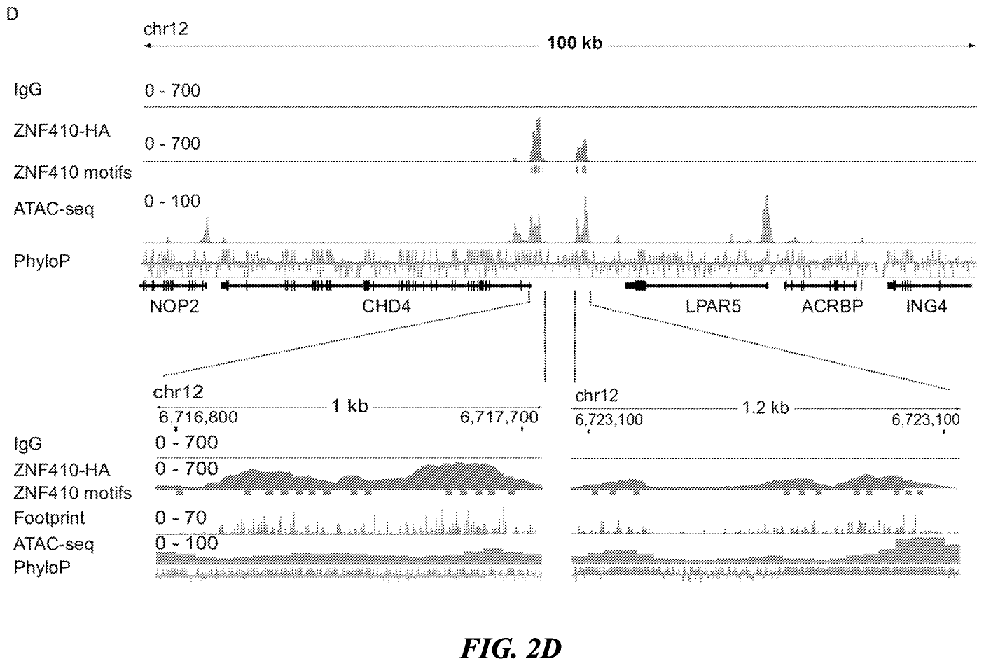

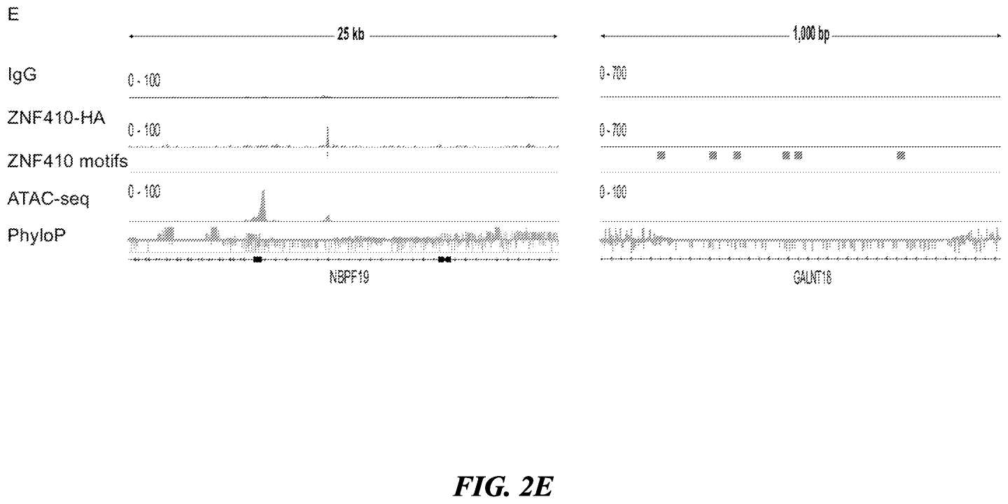

A dense mutagenesis of ZNF410 was performed to identify critical minimal sequences required for function. In this experimental regime, heightened HbF enrichment scores can indicate sequences where not only frameshift but also in-frame mutations are associated with loss-of-function⁶. Heightened HbF enrichment scores were found especially when targeting sequences between exons 6-9 encoding the cluster of five C2H2 zinc fingers of ZNF410 (FIG. 2A). This dependence on its putative DNA binding domain suggested that the DNA-binding function of ZNF410 might be critical for its role in HbF repression.

A CUT&RUN was performed to investigate the chromatin occupancy of ZNF410. Initially an HA antibody was used to probe for epitope tagged ZNF410. Unlike typical DNA binding transcription factors which show tens of thousands of binding sites genome wide, ZNF410 showed highly restricted chromatin occupancy. With standard peak calling parameters, 1750 peaks were found, but most of these had marginal enrichment of ZNF410-HA signal compared to an IgG control. An inflection point in which the 35 peaks with HA/IgG enrichment was observed over 5-fold showed substantially more enrichment than the remaining peaks. The top two peaks were found at the CHD4 locus, one at the promoter (48-fold enrichment) and the other 6 kb upstream of the promoter at a region of open chromatin (78-fold enrichment, FIG. 2B, D). This latter element is subsequently referred to as the CHD4-6 kb enhancer. The third most enriched peak was in an intron of NBPF19, with ˜11 fold enrichment (FIG. 2E).

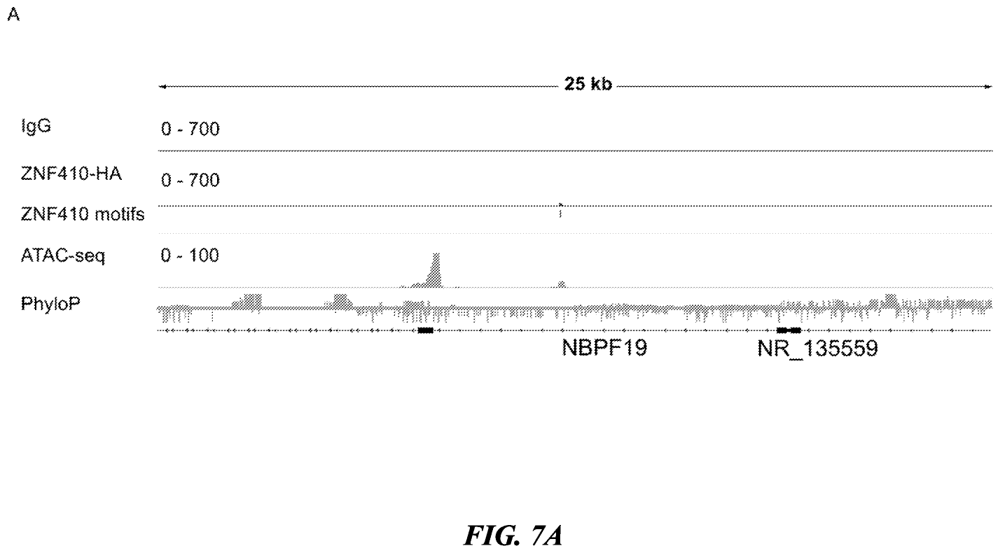

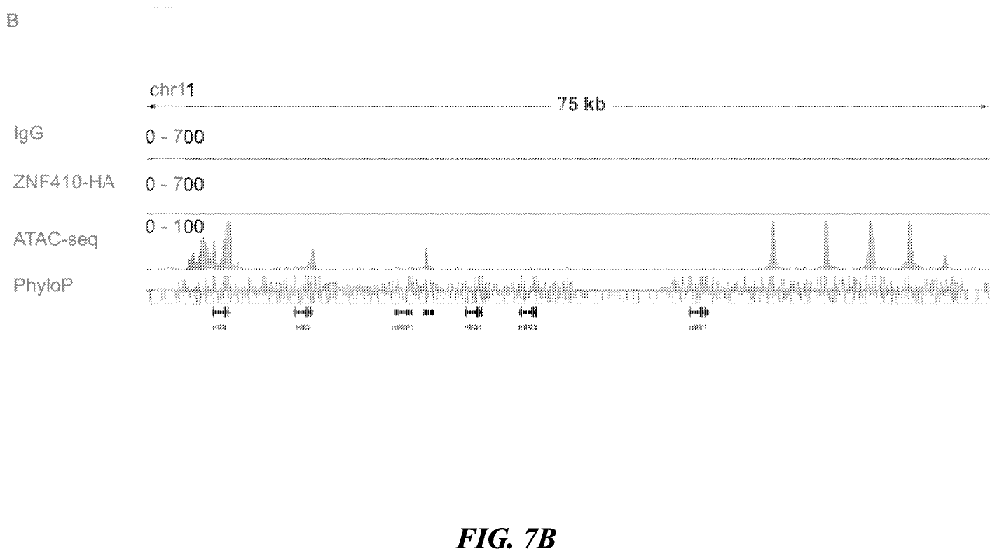

The ZNF410 binding motif has previously been described by high-throughput SELEX using expression of the DNA-binding domain in 293FT cells¹¹. This motif instance was searched across the genome. In addition to scattered single instances of this motif, a striking cluster of these motifs at CHD4 were observed, with numerous motif instances found at both the promoter (16 motifs) and the −6 kb enhancer (11 motifs, FIG. 2C, D). The entire genome was scanned for the ZNF410 binding motif, dividing the genome into 3 kb windows with 100 bp of overlap. 4442 genomic windows had 1 motif instance and 17 windows had 2 motif instances (FIG. 2C). Only 3 windows had more than 2 motif instances, of which 2 were the aforementioned CHD4 elements. Six motif instances within a window at a GALNT18 intron were observed, although neither ZNF410 occupancy nor chromatin accessibility at this locus in erythroid precursors (FIG. 2E). Known HbF repressing TFs like BCL11A and ZBTB7A act by binding to proximal promoter elements at the fetal γ-globin HBG1 and HBG2 genes. In contrast chromatin occupancy of ZNF410 was not observed at the HBB or HBA gene clusters in HUDEP-2 cells (FIG. 7B).

ZNF410 Regulates HbF Through CHD4

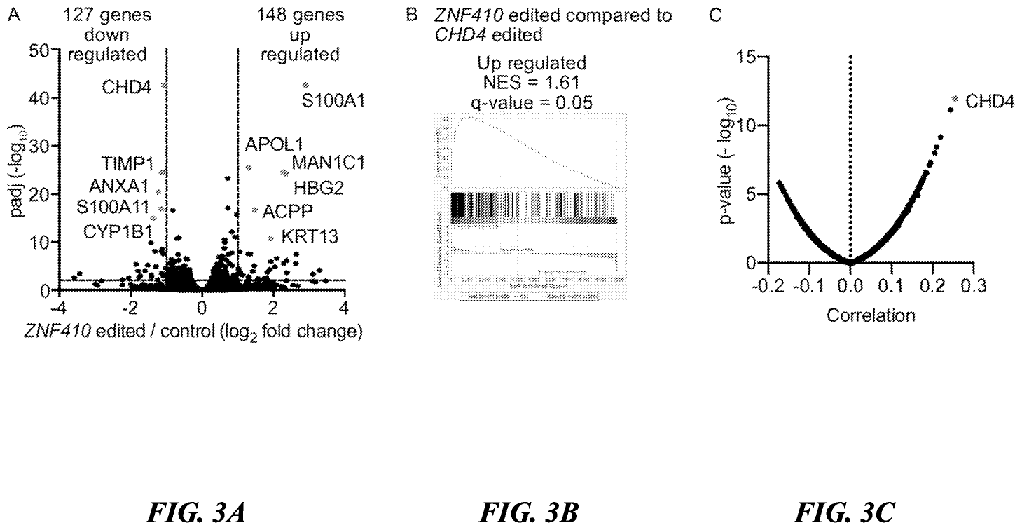

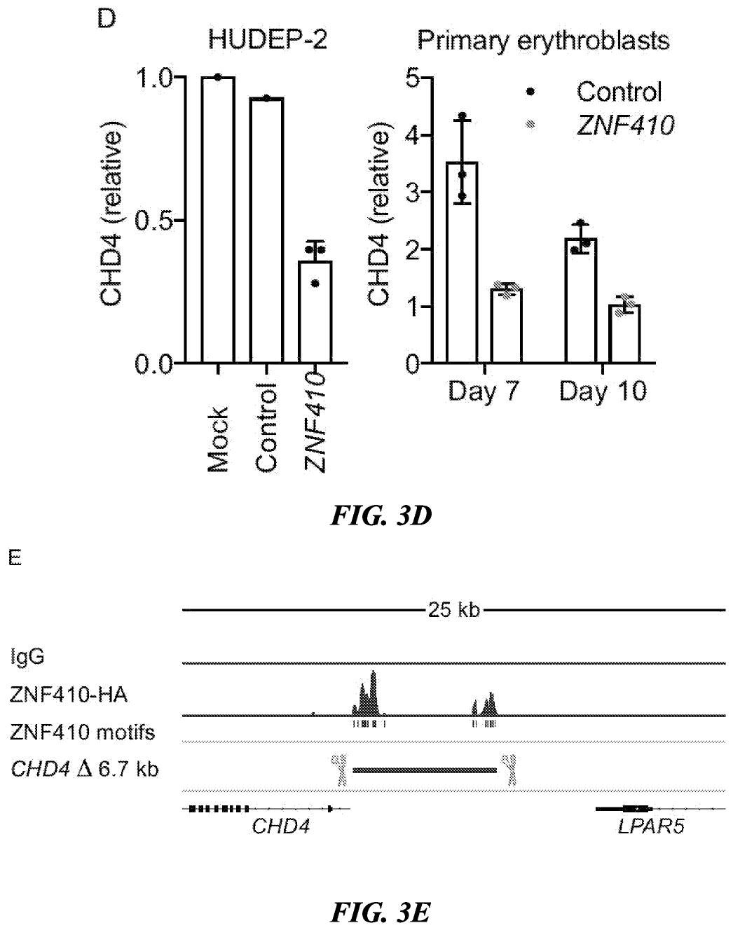

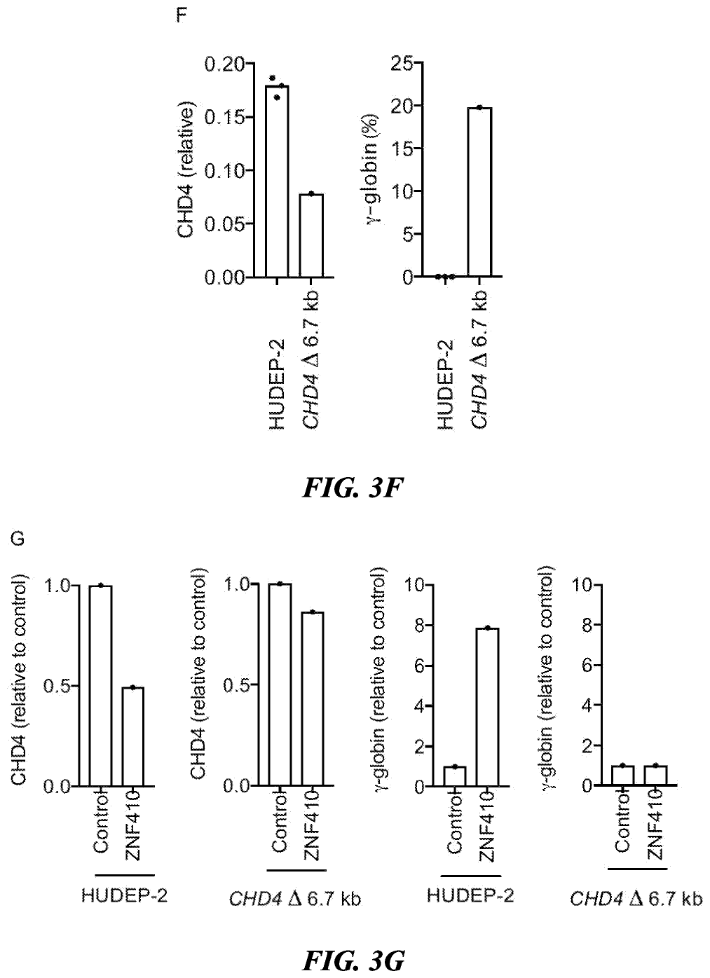

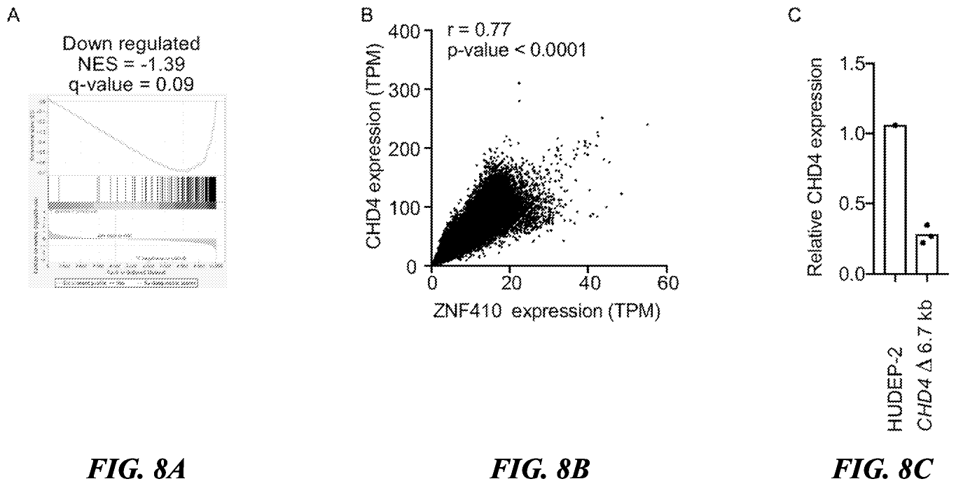

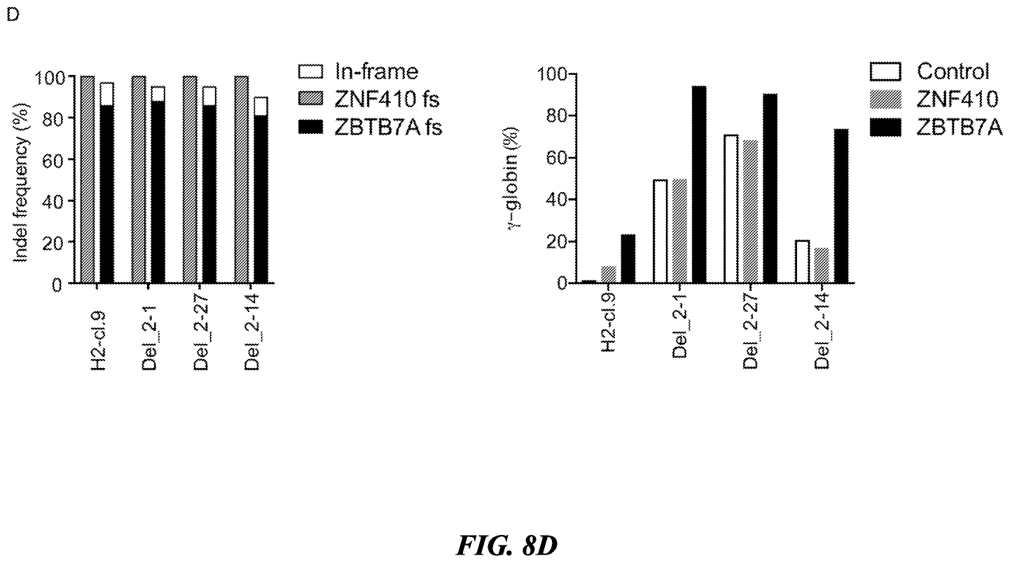

These results suggested that ZNF410 mainly binds to CHD4. We performed RNA-seq of HUDEP-2 cells edited at ZNF410 to measure gene expression changes (FIG. 3A). At an adjusted p-value of 0.01, 275 differentially expressed genes were discovered. CHD4 was the most significantly downregulated gene upon ZNF410 editing (L2FC −1.07, padj 2.27×10-43). HBG2 was the 4th most significant upregulated gene (L2FC 2.35, padj 5.93×10-25). Gene set enrichment analysis was performed to compare the gene expression changes upon ZNF410 editing to those after CHD4 editing. It was discovered that genes differentially expressed after ZNF410 editing were enriched in those differentially expressed after CHD4 editing (for upregulated genes, NES 1.61, q 0.05; for downregulated genes, NES −1.39, q 0.09; FIG. 3B, FIG. 8A). These results indicate that a major function of ZNF410 across a wide set of cellular contexts is to control the expression of CHD4. A dataset of 558 cell lines were evaluated to identify genes with a similar pattern of dependency as ZNF410. It was discovered that CHD4 was the most similarly codependent gene across cell lines, indicating a pervasive relationship between ZNF410 and CHD4 (FIG. 8B-8C)

Changes in CHD4 expression were validated after ZNF410 editing by RT-qPCR in HUDEP-2 cells and primary erythroblasts. CHD4 mRNA expression was reduced by 57% after ZNF410 editing (FIG. 3D). To test the requirement of ZNF410 binding on CHD4 expression, a HUDEP-2 cell clone was generated in which the two upstream ZNF410 motif clusters at CHD4 were both deleted by paired genomic cleavages separated by 6.7 kb (FIG.

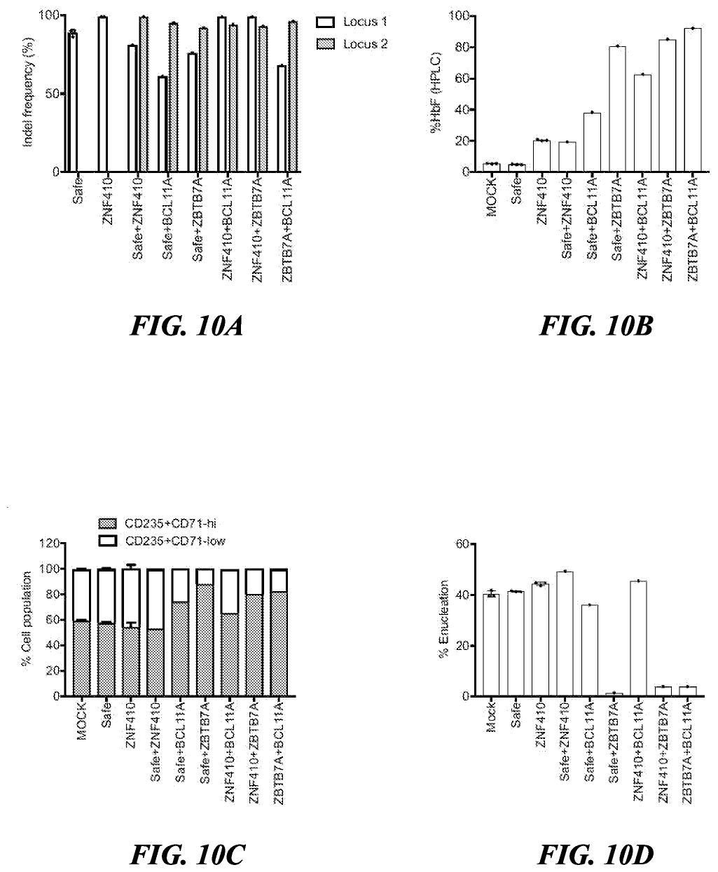

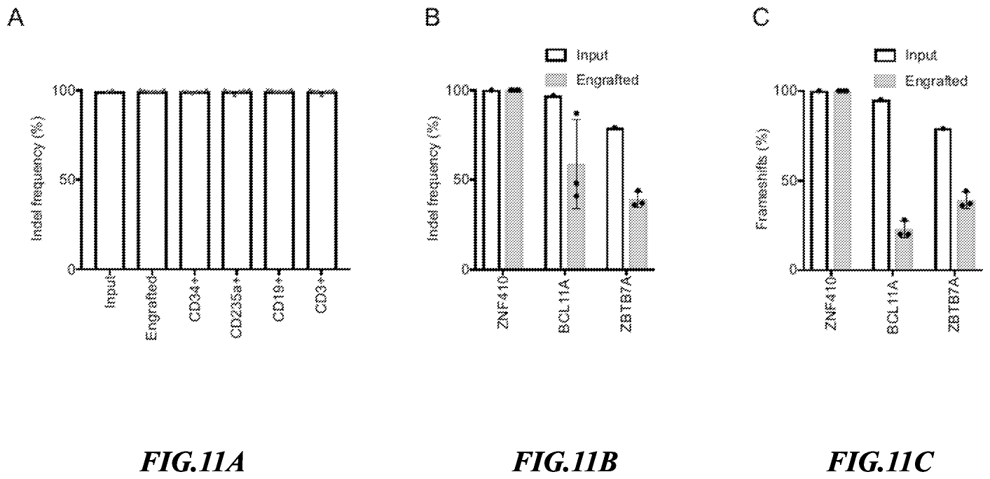

Four biallelically deleted HUDEP-2 clones were isolated. It was found that CHD4 expression decreased by 56% after deletion of the upstream elements similar to that observed after editing ZNF410 itself (FIG. 3F). Consistent with reduced expression of CHD4, -globin was robustly induced (FIG. 3F, FIG. 8D). To test if the effects of ZNF410 on CHD4 required these binding elements, ZNF410 was edited in the element deletion clone. A change in CHD4 expression upon ZNF410 editing in the absence of the upstream elements was no longer observed, suggesting that the control of CHD4 expression requires these elements (FIG. 3G). The absence of γ-globin induction in CHD4 element deleted cells upon ZNF410 editing by RT-qPCR was observed (FIG. 3G, FIG. 8D). In contrast, γ-globin increased in these same cells upon ZBTB7A editing, indicating the cells were competent for further γ-globin induction (FIG. 10A-10D)

Robust editing efficiency was achieved at both ZNF410 and ZBTB7A (FIG. 8D).

Together these results suggest that essentially all of the effects of ZNF410 on repressing γ-globin act through binding upstream elements and subsequent enhancement of CHD4 expression.

ZNF410 is a Non-Essential Gene

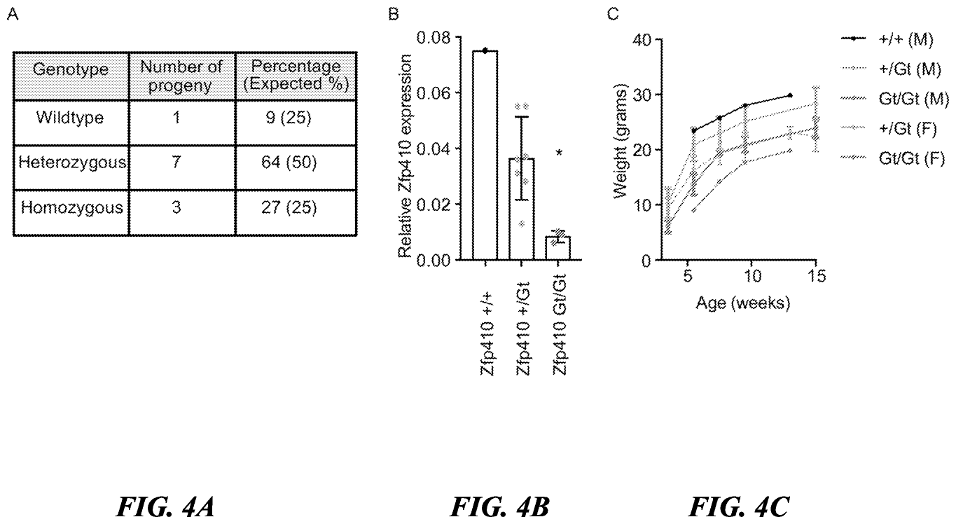

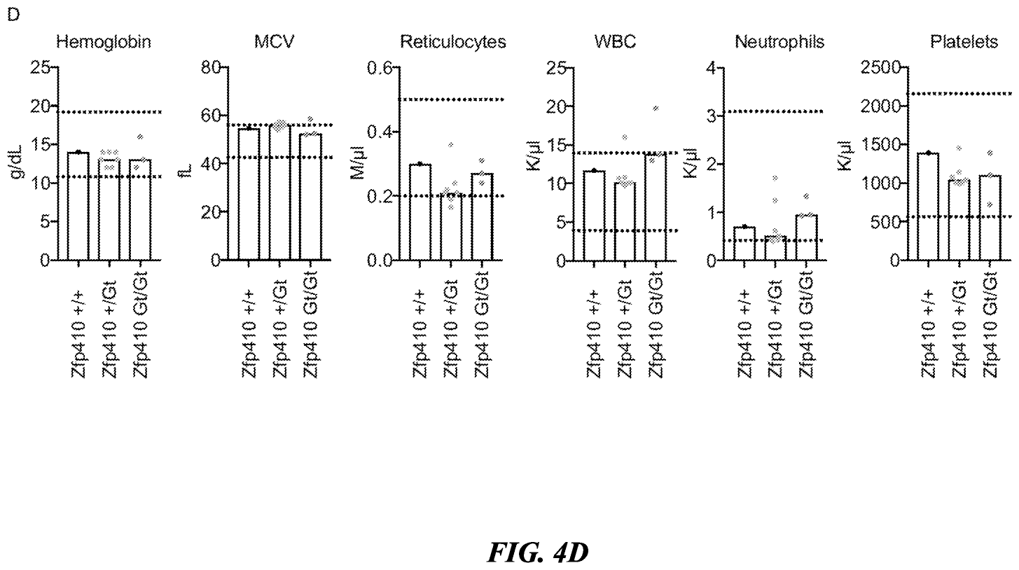

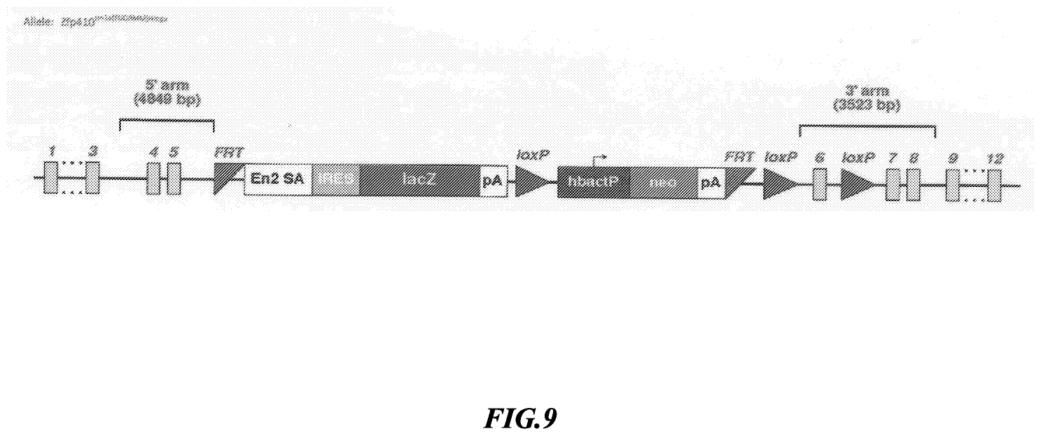

To evaluate the requirement for ZNF410 in normal development and homeostasis, the functional requirement of the mouse ortholog Zfp410 in mice was evaluated with a loss-of-function allele. Zfp410 and ZNF410 share 94% amino acid identity, including 98% at the cluster of 5 ZnFs¹⁰. Mouse embryonic stem cells that are heterozygous for a Zfp410 gene trap allele (Gt) from the European Mouse Mutant Cell Repository (EuMMCR) were obtained. In this allele, the targeting cassette is inserted in intron 5 thus disrupting expression of full-length Zfp410 (FIG. 9). Of note, exons 6-9 encode the five ZnFs. Heterozygous mice were derived with germline transmission of this allele. Although the sample size is currently small, from Zfp410+/Gt heterozygote intercrosses, 3 Zfp410^{Gt/Gt} homozygotes out of 11 live births were observed, consistent with expected Mendelian transmission (FIG. 4A). 4410 expression was reduced by 48% in Zfp410^{+/Gt} heterozygous mice, and by 90% in Zfp410^{Gt/Gt} homozygous mouse blood (FIG. 4B). The Zfp410^{Gt/Gt} homozygotes showed moderately reduced body weight compared to heterozygotes or wt mice (FIG. 4C), but otherwise appeared healthy and active. Analysis of complete blood counts showed apparently unremarkable hematologic parameters in Zfp410Gt/Gt homozygous mice, including no evidence of anemia or hemolysis (FIG. 4D). The absence of a severe phenotype of constitutive Zpf410 loss-of-function is notable in comparison to other HbF regulators. For example, Bcl11a deficient mice experience perinatal lethality¹², Zbtb7a deficient mice mid-gestation embryonic lethality due to anemia¹³, and Chd4 deficient mice pre-implantation embryonic lethality¹⁴. Together these results suggest that ZNF410 is an evolutionarily conserved HbF repressor that is not essential for vertebrate survival.

ZNF410 Appears Dispensable for Human Erythropoiesis and Hematopoiesis

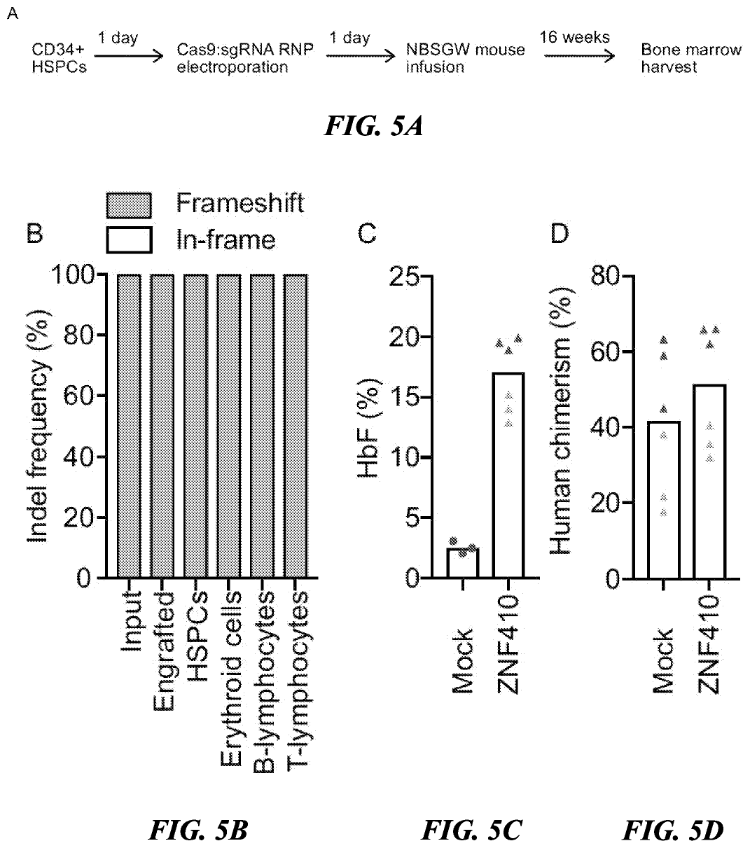

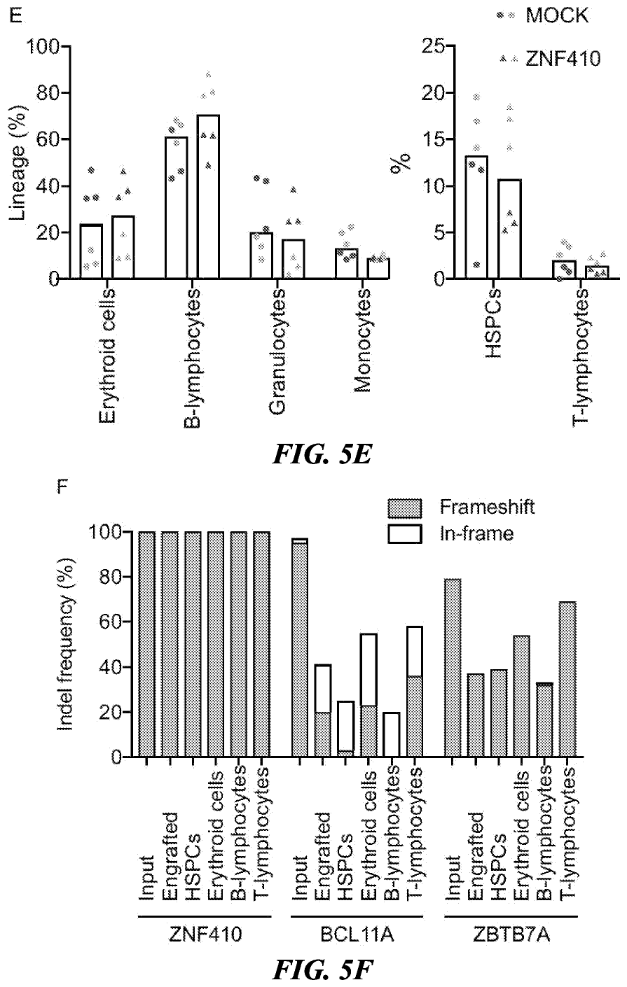

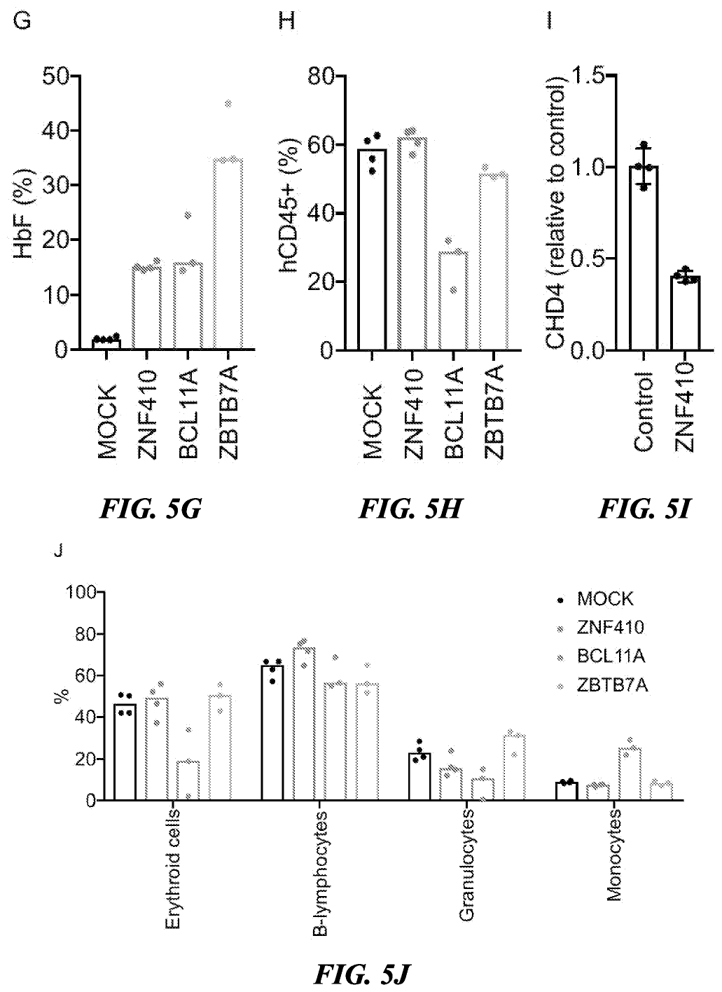

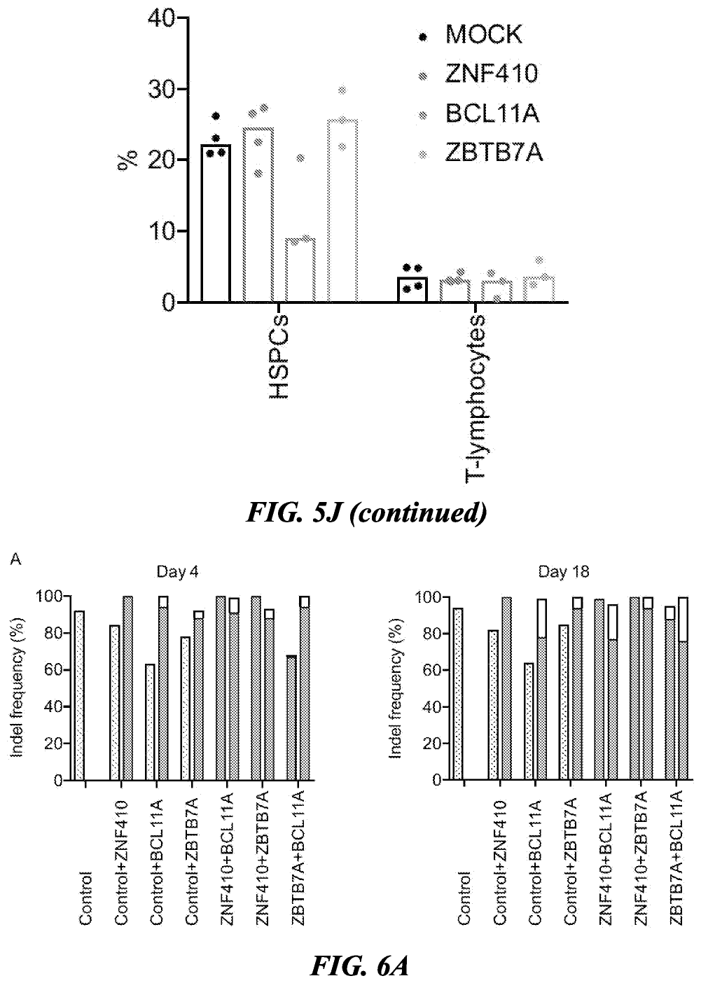

To evaluate the role of ZNF410 in human hematopoiesis, gene editing of ZNF410 was performed in primary human hematopoietic stem and progenitor cells (HSPCs). A 3×NLS-SpCas9 and sgRNA as ribonucleoprotein (RNP) was electroporated into CD34+ HSPCs from a healthy donor and achieved nearly 100% indels (FIG. 5A-5B). Since 94% of these indels were +1 insertions and the remaining were +2 insertions, nearly all cells in the population were comprised of biallelic ZNF410 knockouts.

To test the role of ZNF410 more broadly in hematopoiesis, we performed xenotransplantation of edited HSPCs to immunodeficient NBSGW mice (FIG. 5A). NBSGW mice support multilineage (lymphoid, myeloid, and erythroid) human engraftment in absence of conditioning therapy¹⁵. After 16 weeks we analyzed bone marrow from engrafted recipients. We observed similar human hematopoietic engraftment following ZNF410 or control gene editing (FIG. 5D). ZNF410 indels were observed at 100% in total BM human hematopoietic cells as compared to 100% in the input cell product, with all indels comprised by +1 insertion frameshift alleles (FIG. 5B). A similar distribution of multilineage hematopoietic reconstitution in ZNF410 or control edit recipients was observed, including B-lymphocyte, T-lymphocyte, granulocyte, monocyte, and erythroid contributions (FIG. 5E). Engrafting HSPCs were also similar between groups. Surprisingly, CHD4 expression was decreased by −59% in human erythroid cells sorted from bone marrow, substantiating in vitro results (FIG. 5I). The level of HbF as measured by HPLC from engrafting human erythrocytes was −3% in controls and −17% in ZNF410 edited recipients (FIG. 5C).

For comparison, xenotransplant experiments with BCL11A and ZBTB7A edited HSPC were performed. Consistent with the known role of BCL11A in supporting HSC self-renewal reduced human chimerism was observed in the bone marrow of recipients of BCL11A edited HSPCs after 16 weeks (FIG. 511), reduced fraction of BCL11A edits compared to input cell product, and reduced fraction of frameshift alleles compared to total edits (FIG. 5F). For ZBTB7A, the fraction of engrafting human hematopoietic cells was similar to controls (FIG. 511), but still the fraction of total edits declined as did the fraction of frameshift alleles (FIG. 5F). Together these results suggest HSPCs bearing BCL11A and ZBTB7A loss-of-function alleles are under negative selective pressure following hematopoietic reconstitution, whereas ZNF410 edited cells do not show evidence of selection.

ZNF410 is a Special DNA-Binding Protein

Dense mutagenesis of ZNF410 was performed to identify critical minimal sequences required for function. In this experimental regime, heightened HbF enrichment scores can indicate sequences where not only frameshift but also in-frame mutations are associated with loss-of-function⁶. We found heightened HbF enrichment scores especially when targeting sequences between exons 6-9 encoding the cluster of five C2H2 zinc fingers of ZNF410 (FIG. 10A). This dependence on its putative DNA binding domain suggested that the DNA-binding function of ZNF410 might be critical for its role in HbF repression.

We performed CUT&RUN to investigate the chromatin occupancy of ZNF410. Initially we used an HA antibody to probe for epitope tagged ZNF410. Unlike typical DNA binding transcription factors which show tens of thousands of binding sites genome wide, ZNF410 showed highly restricted chromatin occupancy. With standard peak calling parameters, we found 1750 peaks, but most of these had marginal enrichment of ZNF410-HA signal compared to an IgG control. An inflection point was observed in which the 35 peaks with HA/IgG enrichment over 5-fold showed substantially more enrichment than the remaining peaks. The top two peaks were found at the CHD4 locus, one at the promoter (48-fold enrichment) and the other 6 kb upstream of the promoter at a region of open chromatin (78-fold enrichment, FIG. 10B-10D). This latter element we subsequently refer to as the CHD4-6 kb enhancer. The third most enriched peak was in an intron of NBPF19, with ˜11 fold enrichment (FIG. 10E).

The ZNF410 binding motif has previously been described by high-throughput SELEX using expression of the DNA-binding domain in 293FT cells¹¹. The motif instance was searched across the genome. In addition to scattered single instances of this motif, a striking cluster of these motifs at CHD4 was observed with numerous motif instances found at both the promoter (16 motifs) and the −6 kb enhancer (11 motifs, FIG. 10C-10D). The entire genome was scanned for the ZNF410 binding motif, dividing the genome into 3 kb windows with 100 bp of overlap. 4442 genomic windows had 1 motif instance and 17 windows had 2 motif instances (FIG. 10C). Only 3 windows had more than 2 motif instances, of which 2 were the aforementioned CHD4 elements. 6 motif instances were observed within a window at a GALNT18 intron, although neither ZNF410 occupancy nor chromatin accessibility was observed at this locus in erythroid precursors (FIG. 10E). Known HbF repressing TFs like BCL11A and ZBTB7A act by binding to proximal promoter elements at the fetal-globin HBG1 and HBG2 genes. In contrast, chromatin occupancy of ZNF410 was not observed at the HBB or HBA gene clusters in HUDEP-2 cells (FIG. 7B).

### Discussion

The advances in knowledge of the molecular details of hemoglobin switching have begun to bear fruits in the form of novel autologous therapies1. In clinical trials or late preclinical development are a host of HSC-based therapies that reduce the expression of BCL11A in erythroid cells or prevent its binding to HBG1/2 promoter sequences. However, the clinical unmet need remains vast, with −300,000 infants estimated to be born each year worldwide with sickle cell disease, not to mention tens of thousands more with severe forms of -thalassemia. The feasibility, in terms of cost and infrastructure, to scale up autologous cell-based therapies is uncertain. Furthermore, the toxicity of myeloablative transplantation will likely render these therapies out of reach for many patients.

The most realistic near-term hope to develop scalable therapies to address the root cause of these diseases would be through pharmacotherapy. Drugs that could interrupt molecular vulnerabilities required for adult erythroid cells to maintain fetal globins in a silenced state are greatly needed. These could complement or even supplant existing treatments like hydroxyurea 16. BCL11A itself would certainly represent a preeminent target. Its roles in erythropoiesis besides HbF silencing are modest. However, BCL11A plays essential roles in various hematopoietic lineages, including in B-lymphocytes, dendritic cells and hematopoietic stem cells. In addition, it has functions beyond hematopoiesis not only in the central nervous system but also in breast and pancreatic cells. Another exciting target would be ZBTB7A given its potent role in HbF repression. However, ZBTB7A is required for terminal erythropoiesis and germinal center B cell maturation and plays important roles in T-lymphocytes, osteoclasts and HSCs17. A specific NuRD subcomplex including CHD4, GATAD2A, MBD2, MTA2 and HDAC2 is required for HbF silencing6,18. Targeting NuRD including key protein-protein interactions appears promising but would need to consider the numerous gene expression programs that depend on this chromatin complex. For most of the known HbF regulators, their pleiotropic roles could yield potential on-target liabilities with narrow therapeutic index even if rational targeting approaches could be devised.

ZNF410 is identified herein as a novel HbF repressor that acts specifically to enhance the expression of CHD4. Complete knockout of ZNF410 is well-tolerated, apparently since the remaining level of CHD4 is sufficient to maintain cellular functions. Zfp410 mutant mice survive to adulthood and ZNF410 knockout HSPCs demonstrate no defects in erythroid maturation or hematopoietic reconstitution. Furthermore, it was discovered that ZNF410 and BCL11A inhibition appear additive with respect to HbF induction suggesting that ZNF410 targeting can be combined with other approaches. These results are consistent with the notion that CHD4 plays both BCL11A dependent and BCL11A independent roles in HbF silencing, with the latter potentially mediated through ZBTB7A. Traditionally TFs have been considered undruggable targets. However, the example of small molecules binding and resulting in specific degradation of zinc finger proteins like IKZF1 has encouraged the development of ligands to modulate DNA-binding factors^{19,20}.

ZNF410 appears to represent a special form of gene regulator. Conventional DNA-binding TFs bind and directly control the expression of thousands of genomic targets. In contrast, ZNF410 shows unique binding to CHD4 unmatched among its binding targets. This exquisite specificity is achieved through a remarkable clustering of 27 ZNF410 binding sites at the CHD4 promoter and −6 kb enhancer, a density unlike anywhere else in the genome. Both ZNF410 itself and its two target elements at CHD4 are highly conserved across vertebrates. Another example of clustered homotypic TF binding sites required for gene expression are the binding sites for ZFP64 at the MLL gene promoter, activating the expression of the chromatin regulator MLL²². Chromatin regulatory complexes are abundant nuclear factors that must be maintained at precise levels to maintain proper gene regulation particularly during development. CHD4 is an especially abundant nuclear protein in erythroid precursors²¹. Haploinsufficiency of MLL or CHD4 each cause impaired intellectual development and congenital anomalies²³⁻²⁵. There are greater than a thousand putative DNA-binding TFs, for many of which the genomic binding sites and regulons are poorly characterized²⁶. ZNF410 can be emblematic of a class of TFs relying on homotypic motif clusters with limited gene targets that are devoted to maintenance of core gene regulatory mechanisms.

In summary, ZNF410 was identified as a TF that represses HbF level in adult-stage cells by devoted maintenance of NuRD subcomplex levels through binding an extraordinary cluster of sites upstream of CHD4.

### REFERENCES

1) Orkin, S. H. & Bauer, D. E. Emerging Genetic Therapy for Sickle Cell Disease. Annu. Rev. Med. 70, 257-271 (2019).
2) Piel, F. B., Steinberg, M. H. & Rees, D. C. Sickle Cell Disease. The New England journal of medicine vol. 377 305 (2017).
3) Weatherall, D. J. The Evolving Spectrum of the Epidemiology of Thalassemia. Hematol. Oncol. Clin. North Am. 32, 165-175 (2018).
4) Sankaran, V. G. et al. Human Fetal Hemoglobin Expression Is Regulated by the Developmental Stage-Specific Repressor BCL11A. Science 322, 1839-1842 (2008).
5) Masuda, T. et al. Transcription factors LRF and BCL11A independently repress expression of fetal hemoglobin. Science 351, 285-289 (2016).
6) Sher, F. et al. Rational targeting of a NuRD subcomplex guided by comprehensive in situ mutagenesis. Nat. Genet. 51, 1149-1159 (2019).
7) Martyn, G. E. et al. Natural regulatory mutations elevate fetal globin via disruption of BCL11A or ZBTB7A binding. Nat. Genet. 50, 498-503 (2018).
8) Liu, N. et al. Direct Promoter Repression by BCL11A Controls the Fetal to Adult Hemoglobin Switch. Cell 173, 430-442.e17 (2018).
9) Xu, J. et al. Corepressor-dependent silencing of fetal hemoglobin expression by BCL11A. Proceedings of the National Academy of Sciences 110, 6518-6523 (2013).
10) Benanti, J. A., Williams, D. K., Robinson, K. L., Ozer, H. L. & Galloway, D. A. Induction of extracellular matrix-remodeling genes by the senescence-associated protein APA-1. Mol. Cell. Biol. 22, 7385-7397 (2002).
11) Jolma, A. et al. DNA-binding specificities of human transcription factors. Cell 152, 327-339 (2013).
12) Liu, P. et al. Bcl11a is essential for normal lymphoid development. Nat. Immunol. 4, 525-532 (2003).
13) Maeda, T. et al. LRF Is an Essential Downstream Target of GATA1 in Erythroid Development and Regulates BIM-Dependent Apoptosis. Dev. Cell 17, 527-540 (2009).
14) O'Shaughnessy-Kirwan, A., Signolet, J., Costello, I., Gharbi, S. & Hendrich, B. Constraint of gene expression by the chromatin remodelling protein CHD4 facilitates lineage specification. Development 142, 2586-2597 (2015).
15) McIntosh, B. E. et al. Nonirradiated NOD,B6. SCID Il2rgamma−/− kitW41/W41 (NBSGW) mice support multilineage engraftment of human hematopoietic cells. Stem Cell Reports 4, 171-180 (2015).
16) John, C. C. et al. Hydroxyurea Dose Escalation for Sickle Cell Anemia in Sub-Saharan Africa. N. Engl. J. Med. 382, 2524-2533 (2020).
17) Maeda, T. Regulation of hematopoietic development by ZBTB transcription factors. Int. J. Hematol. 104, 310-323 (2016).
18) Yu, X. et al. Disruption of the MBD2-NuRD complex but not MBD3-NuRD induces high level HbF expression in human erythroid cells. Haematologica 104, haematol.2018.210963 (2019).
19) Krönke, J. et al. Lenalidomide causes selective degradation of IKZF1 and IKZF3 in multiple myeloma cells. Science 343, 301-305 (2014).
20) Lu, G. et al. The myeloma drug lenalidomide promotes the cereblon-dependent destruction of Ikaros proteins. Science 343, 305-309 (2014).
21) Gillespie, M. A. et al. Absolute Quantification of Transcription Factors Reveals Principles of Gene Regulation in Erythropoiesis. Mol. Cell 78, 960-974.e11 (2020).
22) Lu, B. et al. A Transcription Factor Addiction in Leukemia Imposed by the MLL Promoter Sequence. Cancer Cell 34, 970-981.e8 (2018).
23) Jones, W. D. et al. De novo mutations in MLL cause Wiedemann-Steiner syndrome. Am. J. Hum. Genet. 91, 358-364 (2012).
24) Sifrim, A. et al. Distinct genetic architectures for syndromic and nonsyndromic congenital heart defects identified by exome sequencing. Nat. Genet. 48, 1060-1065 (2016).
25) Weiss, K. et al. De Novo Mutations in CHD4, an ATP-Dependent Chromatin Remodeler Gene, Cause an Intellectual Disability Syndrome with Distinctive Dysmorphisms. Am. J. Hum. Genet. 99, 934-941 (2016).
26) Lambert, S. A. et al. The Human Transcription Factors. Cell 172, 650-665 (2018).

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of increasing fetal hemoglobin level in a subject, the method comprising: administering to a subject in need thereof an agent that decreases the level or activity of zinc finger 410 (ZNF410).

2. The method of claim 1, wherein the subject has a hemoglobinopathy.

3. A method of treating a hemoglobinopathy in a subject, the method comprising: administering to a subject in need thereof an agent that decreases the level or activity of ZNF410.

4. The method of claim 1, wherein the agent is a nucleic acid comprising a nucleotide sequence complementary to at least a portion of a nucleic acid encoding ZNF410, an anti-ZNF410 antibody, a zinc finger inhibitor, or a dominant negative ZNF410 polypeptide.

5. The method of claim 4, wherein the nucleic acid agent is a guide RNA, a sgRNA, an siRNA, a shRNA, an antisense oligonucleotide, an aptamer, a ribozyme, a DNAzyme, or a microRNA.

6. The method of claim 4 or 5, wherein the agent is a nucleic acid comprising a nucleotide sequence selected from SEQ ID NO: 1-SEQ ID NO: 183.

7. The method of claim 6, wherein the agent is a nucleic acid comprising a nucleotide sequence selected from SEQ ID NO: 1-SEQ ID NO: 169.

8. The method of claim 7, wherein the agent is a nucleic acid comprising a nucleotide sequence GTACAGTTGAAGGTTGTGAC (SEQ ID NO: 19).

9. The method of claim 4, wherein the agent is an anti-ZNF410 antibody selected from anti-ZNF410 antibody HPA002871, anti-ZNF410 antibody SAB2104187, anti-ZNF410 antibody SAB1407818, anti-ZNF410 antibody NBP2-21008, anti-ZNF410 antibody ABIN2777798, anti-ZNF410 antibody ABIN931225, anti-ZNF410 antibody ABIN1882025, anti-ZNF410 antibody ABIN2155007, anti-ZNF410 antibody ABIN5535398, anti-ZNF410 antibody ABIN6742349, anti-ZNF410 antibody ABIN528331, anti-ZNF410 antibody ABIN2687242, anti-ZNF410 antibody ABIN5516407, ZNF410 Polyclonal antibody, ZNF410 antibody N1C1, ZNF410 antibody MBS8501808, ZNF410 antibody MB S9125687, Proteintech #14529-1-AP and RRID:AB_2257520.

10. The method of claim 4, wherein the zinc finger inhibitor is a zinc finger ejector compound.

11. The method of claim 10, wherein the zinc finger ejector compound is selected from the group consisting of azodicarbonamide (ADA), 3-nitrosobenzamide (NOBA), 6-nitroso-1,2-benzopyrone (NOBP), 2,2′-di-thiobisbenzamide (DIBA), mercaptobenzamides, Pyridinioalkanoyl thiolesters (PATES), and bis-thiadizolbenzene-1,2-diamine.

12. The method of claim 2, wherein the hemoglobinopathy is a β-hemoglobinopathy.

13. The method of claim 12, wherein the hemoglobinopathy is selected from the group consisting of: sickle cell disease; sickle cell anemia; sickle-hemoglobin C disease (HbSC); sickle beta-plus-thalassemia (HbS/β+); sickle beta-zero-thalassemia (HbS/β0); and β-thalassemia.

14. The method of claim 1, wherein the subject is a mammal.

15. The method of claim 14, wherein the subject is human.

16. A synthetic nucleic acid molecule comprising a nucleotide sequence selected from the group consisting of SEQ ID NO: 1-SEQ ID NO: 183.

17.-27. (canceled)

28. A composition comprising a synthetic nucleic acid molecule of claim 16.

29. The composition of claim 28, wherein the composition further comprises a nuclease enzyme.

30.-34. (canceled)

35. A vector comprising the synthetic nucleic acid molecule of claim 16.

36. The vector of claim 35, wherein the vector further comprises a polynucleotide comprising a nucleotide sequence encoding a nuclease enzyme

37.-43. (canceled)


SEQUENCE LISTING (194 sequences)

SEQ ID NO: 1 - DNA, 20 bp, Artificial Sequence
  Description of Artificial Sequence Synthetic oligonucleotide
accttctgcaggacagatga

SEQ ID NO: 2 - DNA, 20 bp, Artificial Sequence
  Description of Artificial Sequence Synthetic oligonucleotide
cacatccttgggcttcacaa

SEQ ID NO: 3 - DNA, 20 bp, Artificial Sequence
  Description of Artificial Sequence Synthetic oligonucleotide
gggaagaccttctctcagag

SEQ ID NO: 4 - DNA, 20 bp, Artificial Sequence
  Description of Artificial Sequence Synthetic oligonucleotide
gagctggccatacaaatgtc

SEQ ID NO: 5 - DNA, 20 bp, Artificial Sequence
  Description of Artificial Sequence Synthetic oligonucleotide
ctggaaacctgaagaaccac

SEQ ID NO: 6 - DNA, 20 bp, Artificial Sequence
  Description of Artificial Sequence Synthetic oligonucleotide
gttgtgaccggacatttgta

SEQ ID NO: 7 - DNA, 20 bp, Artificial Sequence
  Description of Artificial Sequence Synthetic oligonucleotide
agaggctgaaggtgcacatg

SEQ ID NO: 8 - DNA, 20 bp, Artificial Sequence
  Description of Artificial Sequence Synthetic oligonucleotide
ggcttcagtcacgggaagga

SEQ ID NO: 9 - DNA, 20 bp, Artificial Sequence
  Description of Artificial Sequence Synthetic oligonucleotide
aggcttcagtcacgggaagg

SEQ ID NO: 10 - DNA, 20 bp, Artificial Sequence
  Description of Artificial Sequence Synthetic oligonucleotide
ctgaggcttcagtcacggga

SEQ ID NO: 11 - DNA, 20 bp, Artificial Sequence
  Description of Artificial Sequence Synthetic oligonucleotide
ccagactcatggcacataaa

SEQ ID NO: 12 - DNA, 20 bp, Artificial Sequence
  Description of Artificial Sequence Synthetic oligonucleotide
gccagactcatggcacataa

SEQ ID NO: 13 - DNA, 20 bp, Artificial Sequence
  Description of Artificial Sequence Synthetic oligonucleotide
ctcaccgatgagtcttgagg

SEQ ID NO: 14 - DNA, 20 bp, Artificial Sequence
  Description of Artificial Sequence Synthetic oligonucleotide
ttgctcaccgatgagtcttg

SEQ ID NO: 15 - DNA, 20 bp, Artificial Sequence
  Description of Artificial Sequence Synthetic oligonucleotide
aaacatctggtggttcactc

SEQ ID NO: 16 - DNA, 20 bp, Artificial Sequence
  Description of Artificial Sequence Synthetic oligonucleotide
tagaatactcagcaaaggaa

SEQ ID NO: 17 - DNA, 20 bp, Artificial Sequence
  Description of Artificial Sequence Synthetic oligonucleotide
aataccacctcaagactcat

SEQ ID NO: 18 - DNA, 20 bp, Artificial Sequence
  Description of Artificial Sequence Synthetic oligonucleotide
cattagccgactgcattctg

SEQ ID NO: 19 - DNA, 20 bp, Artificial Sequence
  Description of Artificial Sequence Synthetic oligonucleotide
gtacagttgaaggttgtgac

SEQ ID NO: 20 - DNA, 20 bp, Artificial Sequence
  Description of Artificial Sequence Synthetic oligonucleotide
ccacagacttggcactgatg

SEQ ID NO: 21 - DNA, 20 bp, Artificial Sequence
  Description of Artificial Sequence Synthetic oligonucleotide
cacctgtgtggatgcgccgg

SEQ ID NO: 22 - DNA, 20 bp, Artificial Sequence
  Description of Artificial Sequence Synthetic oligonucleotide
acacacctgtgtggatgcgc

SEQ ID NO: 23 - DNA, 20 bp, Artificial Sequence
  Description of Artificial Sequence Synthetic oligonucleotide
tcctgaacccggaggaacca

SEQ ID NO: 24 - DNA, 20 bp, Artificial Sequence
  Description of Artificial Sequence Synthetic oligonucleotide
gcacagacagtagcattcca

SEQ ID NO: 25 - DNA, 20 bp, Artificial Sequence
  Description of Artificial Sequence Synthetic oligonucleotide
ggatgcgccggtggttcttc

SEQ ID NO: 26 - DNA, 20 bp, Artificial Sequence
  Description of Artificial Sequence Synthetic oligonucleotide
tcagactgtacagaaatccc

SEQ ID NO: 27 - DNA, 20 bp, Artificial Sequence
  Description of Artificial Sequence Synthetic oligonucleotide
gctactgtctgtgctctcac

SEQ ID NO: 28 - DNA, 20 bp, Artificial Sequence
  Description of Artificial Sequence Synthetic oligonucleotide
cctttcctttgtgaagccca

SEQ ID NO: 29 - DNA, 20 bp, Artificial Sequence
  Description of Artificial Sequence Synthetic oligonucleotide
agcagctgggagtcaagagc

SEQ ID NO: 30 - DNA, 20 bp, Artificial Sequence
  Description of Artificial Sequence Synthetic oligonucleotide
ctgcaggacagatgaaggag

SEQ ID NO: 31 - DNA, 20 bp, Artificial Sequence
  Description of Artificial Sequence Synthetic oligonucleotide
ctttgtgaagcccaaggatg

SEQ ID NO: 32 - DNA, 20 bp, Artificial Sequence
  Description of Artificial Sequence Synthetic oligonucleotide
aaaggaacggccacatcctt

SEQ ID NO: 33 - DNA, 20 bp, Artificial Sequence
  Description of Artificial Sequence Synthetic oligonucleotide
caaaggaacggccacatcct

SEQ ID NO: 34 - DNA, 20 bp, Artificial Sequence
  Description of Artificial Sequence Synthetic oligonucleotide
tgagttagaatccaaaccag

SEQ ID NO: 35 - DNA, 20 bp, Artificial Sequence
  Description of Artificial Sequence Synthetic oligonucleotide
gactgggctcacctctggtt

SEQ ID NO: 36 - DNA, 20 bp, Artificial Sequence
  Description of Artificial Sequence Synthetic oligonucleotide
aagctgactgggctcacctc

SEQ ID NO: 37 - DNA, 20 bp, Artificial Sequence
  Description of Artificial Sequence Synthetic oligonucleotide
gaaccaccagatgttttcgg

SEQ ID NO: 38 - DNA, 20 bp, Artificial Sequence
  Description of Artificial Sequence Synthetic oligonucleotide
agtgaaccaccagatgtttt

SEQ ID NO: 39 - DNA, 20 bp, Artificial Sequence
  Description of Artificial Sequence Synthetic oligonucleotide
gtagcattccatggttcctc

SEQ ID NO: 40 - DNA, 20 bp, Artificial Sequence
  Description of Artificial Sequence Synthetic oligonucleotide
tagcattccatggttcctcc

SEQ ID NO: 41 - DNA, 20 bp, Artificial Sequence
  Description of Artificial Sequence Synthetic oligonucleotide
cctgcttccactctgagaga

SEQ ID NO: 42 - DNA, 20 bp, Artificial Sequence
  Description of Artificial Sequence Synthetic oligonucleotide
aaagcttctatgtgctgcag

SEQ ID NO: 43 - DNA, 20 bp, Artificial Sequence
  Description of Artificial Sequence Synthetic oligonucleotide
gggccaactcctgaacccgg

SEQ ID NO: 44 - DNA, 20 bp, Artificial Sequence
  Description of Artificial Sequence Synthetic oligonucleotide
tgaagtggacaaaaactccg

SEQ ID NO: 45 - DNA, 20 bp, Artificial Sequence
  Description of Artificial Sequence Synthetic oligonucleotide
ctgaagtggacaaaaactcc

SEQ ID NO: 46 - DNA, 20 bp, Artificial Sequence
  Description of Artificial Sequence Synthetic oligonucleotide
tctgaagtggacaaaaactc

SEQ ID NO: 47 - DNA, 20 bp, Artificial Sequence
  Description of Artificial Sequence Synthetic oligonucleotide
ataaaagaagtgctatcact

SEQ ID NO: 48 - DNA, 20 bp, Artificial Sequence
  Description of Artificial Sequence Synthetic oligonucleotide
ccttgggcttcacaaaggaa

SEQ ID NO: 49 - DNA, 20 bp, Artificial Sequence
  Description of Artificial Sequence Synthetic oligonucleotide
tcttttctccccccagctcc

SEQ ID NO: 50 - DNA, 20 bp, Artificial Sequence
  Description of Artificial Sequence Synthetic oligonucleotide
aacaaactgtaccaggagct

SEQ ID NO: 51 - DNA, 20 bp, Artificial Sequence
  Description of Artificial Sequence Synthetic oligonucleotide
gaggctagaatactcagcaa

SEQ ID NO: 52 - DNA, 20 bp, Artificial Sequence
  Description of Artificial Sequence Synthetic oligonucleotide
gtgcacatgaggacccacaa

SEQ ID NO: 53 - DNA, 20 bp, Artificial Sequence
  Description of Artificial Sequence Synthetic oligonucleotide
aaagggcttctctccattgt

SEQ ID NO: 54 - DNA, 20 bp, Artificial Sequence
  Description of Artificial Sequence Synthetic oligonucleotide
taaagggcttctctccattg

SEQ ID NO: 55 - DNA, 20 bp, Artificial Sequence
  Description of Artificial Sequence Synthetic oligonucleotide
tcagaatacgtccatcccat

SEQ ID NO: 56 - DNA, 20 bp, Artificial Sequence
  Description of Artificial Sequence Synthetic oligonucleotide
cagaatacgtccatcccatt

SEQ ID NO: 57 - DNA, 20 bp, Artificial Sequence
  Description of Artificial Sequence Synthetic oligonucleotide
aagcccctgtcccaatggga

SEQ ID NO: 58 - DNA, 20 bp, Artificial Sequence
  Description of Artificial Sequence Synthetic oligonucleotide
ctacaagcccctgtcccaat

SEQ ID NO: 59 - DNA, 20 bp, Artificial Sequence
  Description of Artificial Sequence Synthetic oligonucleotide
tctacaagcccctgtcccaa

SEQ ID NO: 60 - DNA, 20 bp, Artificial Sequence
  Description of Artificial Sequence Synthetic oligonucleotide
atgtgccatgagtctggctg

SEQ ID NO: 61 - DNA, 20 bp, Artificial Sequence
  Description of Artificial Sequence Synthetic oligonucleotide
aaccaccggcgcatccacac

SEQ ID NO: 62 - DNA, 20 bp, Artificial Sequence
  Description of Artificial Sequence Synthetic oligonucleotide
tggtgctgcacacacctgtg

SEQ ID NO: 63 - DNA, 20 bp, Artificial Sequence
  Description of Artificial Sequence Synthetic oligonucleotide
aaggcttctcaaaacagctg

SEQ ID NO: 64 - DNA, 20 bp, Artificial Sequence
  Description of Artificial Sequence Synthetic oligonucleotide
agctgaggaagggacctcct

SEQ ID NO: 65 - DNA, 20 bp, Artificial Sequence
  Description of Artificial Sequence Synthetic oligonucleotide
tgcagtcggctaatgttacc

SEQ ID NO: 66 - DNA, 20 bp, Artificial Sequence
  Description of Artificial Sequence Synthetic oligonucleotide
gttcctccgggttcaggagt

SEQ ID NO: 67 - DNA, 20 bp, Artificial Sequence
  Description of Artificial Sequence Synthetic oligonucleotide
cattctgaggcttcagtcac

SEQ ID NO: 68 - DNA, 20 bp, Artificial Sequence
  Description of Artificial Sequence Synthetic oligonucleotide
ccttctctcagagtggaagc

SEQ ID NO: 69 - DNA, 20 bp, Artificial Sequence
  Description of Artificial Sequence Synthetic oligonucleotide
ctgaagcctcagaatgcagt

SEQ ID NO: 70 - DNA, 20 bp, Artificial Sequence
  Description of Artificial Sequence Synthetic oligonucleotide
agagcaggagcaaactggtg

SEQ ID NO: 71 - DNA, 20 bp, Artificial Sequence
  Description of Artificial Sequence Synthetic oligonucleotide
tacgtccatcccattgggac

SEQ ID NO: 72 - DNA, 20 bp, Artificial Sequence
  Description of Artificial Sequence Synthetic oligonucleotide
acgtccatcccattgggaca

SEQ ID NO: 73 - DNA, 20 bp, Artificial Sequence
  Description of Artificial Sequence Synthetic oligonucleotide
cgtccatcccattgggacag

SEQ ID NO: 74 - DNA, 20 bp, Artificial Sequence
  Description of Artificial Sequence Synthetic oligonucleotide
acaggagaaaatgtccacct

SEQ ID NO: 75 - DNA, 20 bp, Artificial Sequence
  Description of Artificial Sequence Synthetic oligonucleotide
gcccatcaccagaaccaagg

SEQ ID NO: 76 - DNA, 20 bp, Artificial Sequence
  Description of Artificial Sequence Synthetic oligonucleotide
tgtctgtgctctcactggag

SEQ ID NO: 77 - DNA, 20 bp, Artificial Sequence
  Description of Artificial Sequence Synthetic oligonucleotide
tgagcacttagtgtttgtac

SEQ ID NO: 78 - DNA, 20 bp, Artificial Sequence
  Description of Artificial Sequence Synthetic oligonucleotide
gagaaagcatcacctgcagc

SEQ ID NO: 79 - DNA, 20 bp, Artificial Sequence
  Description of Artificial Sequence Synthetic oligonucleotide
agaaagcatcacctgcagct

SEQ ID NO: 80 - DNA, 20 bp, Artificial Sequence
  Description of Artificial Sequence Synthetic oligonucleotide
tcccagctgctcccagctgc

SEQ ID NO: 81 - DNA, 20 bp, Artificial Sequence
  Description of Artificial Sequence Synthetic oligonucleotide
agtcaagagcaggagcaaac

SEQ ID NO: 82 - DNA, 20 bp, Artificial Sequence
  Description of Artificial Sequence Synthetic oligonucleotide
cagctgttttgagaagcctt

SEQ ID NO: 83 - DNA, 20 bp, Artificial Sequence
  Description of Artificial Sequence Synthetic oligonucleotide
agctgttttgagaagccttc

SEQ ID NO: 84 - DNA, 20 bp, Artificial Sequence
  Description of Artificial Sequence Synthetic oligonucleotide
cctcatcagtgccaagtctg

SEQ ID NO: 85 - DNA, 20 bp, Artificial Sequence
  Description of Artificial Sequence Synthetic oligonucleotide
ctcatcagtgccaagtctgt

SEQ ID NO: 86 - DNA, 20 bp, Artificial Sequence
  Description of Artificial Sequence Synthetic oligonucleotide
agaaggtcttcccacagact

SEQ ID NO: 87 - DNA, 20 bp, Artificial Sequence
  Description of Artificial Sequence Synthetic oligonucleotide
ttcagtacccagtaaaaacc

SEQ ID NO: 88 - DNA, 20 bp, Artificial Sequence
  Description of Artificial Sequence Synthetic oligonucleotide
tccttcatctgtcctgcaga

SEQ ID NO: 89 - DNA, 20 bp, Artificial Sequence
  Description of Artificial Sequence Synthetic oligonucleotide
cttttcccacaaccttctgc

SEQ ID NO: 90 - DNA, 20 bp, Artificial Sequence
  Description of Artificial Sequence Synthetic oligonucleotide
ctatgtgctgcagaggctga

SEQ ID NO: 91 - DNA, 20 bp, Artificial Sequence
  Description of Artificial Sequence Synthetic oligonucleotide
gaacaaactgtaccaggagc

SEQ ID NO: 92 - DNA, 20 bp, Artificial Sequence
  Description of Artificial Sequence Synthetic oligonucleotide
aaagaatacctgctcttgtt

SEQ ID NO: 93 - DNA, 20 bp, Artificial Sequence
  Description of Artificial Sequence Synthetic oligonucleotide
ttctagcctccgaaaacatc

SEQ ID NO: 94 - DNA, 20 bp, Artificial Sequence
  Description of Artificial Sequence Synthetic oligonucleotide
tagcctccgaaaacatctgg

SEQ ID NO: 95 - DNA, 20 bp, Artificial Sequence
  Description of Artificial Sequence Synthetic oligonucleotide
accaaataccaattctatcc

SEQ ID NO: 96 - DNA, 20 bp, Artificial Sequence
  Description of Artificial Sequence Synthetic oligonucleotide
ccaaataccaattctatcct

SEQ ID NO: 97 - DNA, 20 bp, Artificial Sequence
  Description of Artificial Sequence Synthetic oligonucleotide
cgggtgaatgtgggtccaga

SEQ ID NO: 98 - DNA, 20 bp, Artificial Sequence
  Description of Artificial Sequence Synthetic oligonucleotide
gctctcgtctcctctccgtc

SEQ ID NO: 99 - DNA, 20 bp, Artificial Sequence
  Description of Artificial Sequence Synthetic oligonucleotide
aggtggtatttaaagtgagc

SEQ ID NO: 100 - DNA, 20 bp, Artificial Sequence
  Description of Artificial Sequence Synthetic oligonucleotide
caagctagaagactctgaag

SEQ ID NO: 101 - DNA, 20 bp, Artificial Sequence
  Description of Artificial Sequence Synthetic oligonucleotide
aatgcaaaaaccagcagcaa

SEQ ID NO: 102 - DNA, 20 bp, Artificial Sequence
  Description of Artificial Sequence Synthetic oligonucleotide
acgggcctcaccattgctgc

SEQ ID NO: 103 - DNA, 20 bp, Artificial Sequence
  Description of Artificial Sequence Synthetic oligonucleotide
ttctcaaaacagctgaggaa

SEQ ID NO: 104 - DNA, 20 bp, Artificial Sequence
  Description of Artificial Sequence Synthetic oligonucleotide
cttctcaaaacagctgagga

SEQ ID NO: 105 - DNA, 20 bp, Artificial Sequence
  Description of Artificial Sequence Synthetic oligonucleotide
agcagcaatcaaactgtcat

SEQ ID NO: 106 - DNA, 20 bp, Artificial Sequence
  Description of Artificial Sequence Synthetic oligonucleotide
tagcagcaatcaaactgtca

SEQ ID NO: 107 - DNA, 20 bp, Artificial Sequence
  Description of Artificial Sequence Synthetic oligonucleotide
cttcttaacctaacaagagc

SEQ ID NO: 108 - DNA, 20 bp, Artificial Sequence
  Description of Artificial Sequence Synthetic oligonucleotide
tccatggttcctccgggttc

SEQ ID NO: 109 - DNA, 20 bp, Artificial Sequence
  Description of Artificial Sequence Synthetic oligonucleotide
catgggccaactcctgaacc

SEQ ID NO: 110 - DNA, 20 bp, Artificial Sequence
  Description of Artificial Sequence Synthetic oligonucleotide
tattctgaacaaactgtacc

SEQ ID NO: 111 - DNA, 20 bp, Artificial Sequence
  Description of Artificial Sequence Synthetic oligonucleotide
aaggagatttaactgaaaga

SEQ ID NO: 112 - DNA, 20 bp, Artificial Sequence
  Description of Artificial Sequence Synthetic oligonucleotide
aaatcattctaactcctcca

SEQ ID NO: 113 - DNA, 20 bp, Artificial Sequence
  Description of Artificial Sequence Synthetic oligonucleotide
tcattctaactcctccaagg

SEQ ID NO: 114 - DNA, 20 bp, Artificial Sequence
  Description of Artificial Sequence Synthetic oligonucleotide
tgaggaagggacctccttgg

SEQ ID NO: 115 - DNA, 20 bp, Artificial Sequence
  Description of Artificial Sequence Synthetic oligonucleotide
ggatagaattggtatttggt

SEQ ID NO: 116 - DNA, 20 bp, Artificial Sequence
  Description of Artificial Sequence Synthetic oligonucleotide
cccaggatagaattggtatt

SEQ ID NO: 117 - DNA, 20 bp, Artificial Sequence
  Description of Artificial Sequence Synthetic oligonucleotide
gcattctgaggcttcagtca

SEQ ID NO: 118 - DNA, 20 bp, Artificial Sequence
  Description of Artificial Sequence Synthetic oligonucleotide
cacatcaggcactgaagaca

SEQ ID NO: 119 - DNA, 20 bp, Artificial Sequence
  Description of Artificial Sequence Synthetic oligonucleotide
tcacatcaggcactgaagac

SEQ ID NO: 120 - DNA, 20 bp, Artificial Sequence
  Description of Artificial Sequence Synthetic oligonucleotide
cactgaagacagggaacgtg

SEQ ID NO: 121 - DNA, 20 bp, Artificial Sequence
  Description of Artificial Sequence Synthetic oligonucleotide
gcactgaagacagggaacgt

SEQ ID NO: 122 - DNA, 20 bp, Artificial Sequence
  Description of Artificial Sequence Synthetic oligonucleotide
ggcactgaagacagggaacg

SEQ ID NO: 123 - DNA, 20 bp, Artificial Sequence
  Description of Artificial Sequence Synthetic oligonucleotide
caggatgaggcagaagattc

SEQ ID NO: 124 - DNA, 20 bp, Artificial Sequence
  Description of Artificial Sequence Synthetic oligonucleotide
aggatgaggcagaagattca

SEQ ID NO: 125 - DNA, 20 bp, Artificial Sequence
  Description of Artificial Sequence Synthetic oligonucleotide
ctgtgcagctgagccactaa

SEQ ID NO: 126 - DNA, 20 bp, Artificial Sequence
  Description of Artificial Sequence Synthetic oligonucleotide
tgtgcagctgagccactaat

SEQ ID NO: 127 - DNA, 20 bp, Artificial Sequence
  Description of Artificial Sequence Synthetic oligonucleotide
agcaaactactgcccattag

SEQ ID NO: 128 - DNA, 20 bp, Artificial Sequence
  Description of Artificial Sequence Synthetic oligonucleotide
ggttcaaggactctccacca

SEQ ID NO: 129 - DNA, 20 bp, Artificial Sequence
  Description of Artificial Sequence Synthetic oligonucleotide
aaaaaccagcagcaatggtg

SEQ ID NO: 130 - DNA, 20 bp, Artificial Sequence
  Description of Artificial Sequence Synthetic oligonucleotide
tcttaccatcatcaactccc

SEQ ID NO: 131 - DNA, 20 bp, Artificial Sequence
  Description of Artificial Sequence Synthetic oligonucleotide
ttctgggccccttcctcaag

SEQ ID NO: 132 - DNA, 20 bp, Artificial Sequence
  Description of Artificial Sequence Synthetic oligonucleotide
ttgagcttcttttccacttg

SEQ ID NO: 133 - DNA, 20 bp, Artificial Sequence
  Description of Artificial Sequence Synthetic oligonucleotide
atctgtcctgcagaaggttg

SEQ ID NO: 134 - DNA, 20 bp, Artificial Sequence
  Description of Artificial Sequence Synthetic oligonucleotide
tctgtcctgcagaaggttgt

SEQ ID NO: 135 - DNA, 20 bp, Artificial Sequence
  Description of Artificial Sequence Synthetic oligonucleotide
cttttacagttactaaacca

SEQ ID NO: 136 - DNA, 20 bp, Artificial Sequence
  Description of Artificial Sequence Synthetic oligonucleotide
gtccaccttggttctggtga

SEQ ID NO: 137 - DNA, 20 bp, Artificial Sequence
  Description of Artificial Sequence Synthetic oligonucleotide
tccaccttggttctggtgat

SEQ ID NO: 138 - DNA, 20 bp, Artificial Sequence
  Description of Artificial Sequence Synthetic oligonucleotide
aactgcagtaaatccacaag

SEQ ID NO: 139 - DNA, 20 bp, Artificial Sequence
  Description of Artificial Sequence Synthetic oligonucleotide
agagaccactttacctcttg

SEQ ID NO: 140 - DNA, 20 bp, Artificial Sequence
  Description of Artificial Sequence Synthetic oligonucleotide
ctctccaccaaggctgggct

SEQ ID NO: 141 - DNA, 20 bp, Artificial Sequence
  Description of Artificial Sequence Synthetic oligonucleotide
actctccaccaaggctgggc

SEQ ID NO: 142 - DNA, 20 bp, Artificial Sequence
  Description of Artificial Sequence Synthetic oligonucleotide
ctgaaagacggacatgagcg

SEQ ID NO: 143 - DNA, 20 bp, Artificial Sequence
  Description of Artificial Sequence Synthetic oligonucleotide
attttgtttcttccaggtct

SEQ ID NO: 144 - DNA, 20 bp, Artificial Sequence
  Description of Artificial Sequence Synthetic oligonucleotide
tcagctgaagagcccagacc

SEQ ID NO: 145 - DNA, 20 bp, Artificial Sequence
  Description of Artificial Sequence Synthetic oligonucleotide
gctgggaattcatagacacc

SEQ ID NO: 146 - DNA, 20 bp, Artificial Sequence
  Description of Artificial Sequence Synthetic oligonucleotide
gggcagtagtttgcttgaag

SEQ ID NO: 147 - DNA, 20 bp, Artificial Sequence
  Description of Artificial Sequence Synthetic oligonucleotide
ccctttatgtgccatgagtc

SEQ ID NO: 148 - DNA, 20 bp, Artificial Sequence
  Description of Artificial Sequence Synthetic oligonucleotide
gcttaccacagccagactca

SEQ ID NO: 149 - DNA, 20 bp, Artificial Sequence
  Description of Artificial Sequence Synthetic oligonucleotide
ctggacccacattcacccga

SEQ ID NO: 150 - DNA, 20 bp, Artificial Sequence
  Description of Artificial Sequence Synthetic oligonucleotide
gaatgtgggtccagacggag

SEQ ID NO: 151 - DNA, 20 bp, Artificial Sequence
  Description of Artificial Sequence Synthetic oligonucleotide
cttagtgtttgtacaggatg

SEQ ID NO: 152 - DNA, 20 bp, Artificial Sequence
  Description of Artificial Sequence Synthetic oligonucleotide
ggtaagcagtttactacagc

SEQ ID NO: 153 - DNA, 20 bp, Artificial Sequence
  Description of Artificial Sequence Synthetic oligonucleotide
gaaaatgtccaccttggttc

SEQ ID NO: 154 - DNA, 20 bp, Artificial Sequence
  Description of Artificial Sequence Synthetic oligonucleotide
actgcccatcaccagaacca

SEQ ID NO: 155 - DNA, 20 bp, Artificial Sequence
  Description of Artificial Sequence Synthetic oligonucleotide
gtctttcagttaaatctcct

SEQ ID NO: 156 - DNA, 20 bp, Artificial Sequence
  Description of Artificial Sequence Synthetic oligonucleotide
accaggtgatgtgtcacatc

SEQ ID NO: 157 - DNA, 20 bp, Artificial Sequence
  Description of Artificial Sequence Synthetic oligonucleotide
tgctgctactcgtgcacaac

SEQ ID NO: 158 - DNA, 20 bp, Artificial Sequence
  Description of Artificial Sequence Synthetic oligonucleotide
atagacaccaggtttttact

SEQ ID NO: 159 - DNA, 20 bp, Artificial Sequence
  Description of Artificial Sequence Synthetic oligonucleotide
catagacaccaggtttttac

SEQ ID NO: 160 - DNA, 20 bp, Artificial Sequence
  Description of Artificial Sequence Synthetic oligonucleotide
attggtatttggtaggttca

SEQ ID NO: 161 - DNA, 20 bp, Artificial Sequence
  Description of Artificial Sequence Synthetic oligonucleotide
atgaattcccagcccagcct

SEQ ID NO: 162 - DNA, 20 bp, Artificial Sequence
  Description of Artificial Sequence Synthetic oligonucleotide
aaggactctccaccaaggct

SEQ ID NO: 163 - DNA, 20 bp, Artificial Sequence
  Description of Artificial Sequence Synthetic oligonucleotide
aattcccagcccagccttgg

SEQ ID NO: 164 - DNA, 20 bp, Artificial Sequence
  Description of Artificial Sequence Synthetic oligonucleotide
caaggactctccaccaaggc

SEQ ID NO: 165 - DNA, 20 bp, Artificial Sequence
  Description of Artificial Sequence Synthetic oligonucleotide
gatgggcagtcaaaagattc

SEQ ID NO: 166 - DNA, 20 bp, Artificial Sequence
  Description of Artificial Sequence Synthetic oligonucleotide
atgggcagtcaaaagattct

SEQ ID NO: 167 - DNA, 20 bp, Artificial Sequence
  Description of Artificial Sequence Synthetic oligonucleotide
gcctgatgtgacacatcacc

SEQ ID NO: 168 - DNA, 20 bp, Artificial Sequence
  Description of Artificial Sequence Synthetic oligonucleotide
gagaagccttcgggtgaatg

SEQ ID NO: 169 - DNA, 20 bp, Artificial Sequence
  Description of Artificial Sequence Synthetic oligonucleotide
agaagccttcgggtgaatgt

SEQ ID NO: 170 - DNA, 20 bp, Artificial Sequence
  Description of Artificial Sequence Synthetic oligonucleotide
cacctgcagctgggagcagc

SEQ ID NO: 171 - DNA, 20 bp, Artificial Sequence
  Description of Artificial Sequence Synthetic oligonucleotide
acctgcagctgggagcagct

SEQ ID NO: 172 - DNA, 20 bp, Artificial Sequence
  Description of Artificial Sequence Synthetic oligonucleotide
cacctggtgaccatgcagtc

SEQ ID NO: 173 - DNA, 20 bp, Artificial Sequence
  Description of Artificial Sequence Synthetic oligonucleotide
acctggtgaccatgcagtca

SEQ ID NO: 174 - DNA, 20 bp, Artificial Sequence
  Description of Artificial Sequence Synthetic oligonucleotide
tgattgcctccctgactgca

SEQ ID NO: 175 - DNA, 20 bp, Artificial Sequence
  Description of Artificial Sequence Synthetic oligonucleotide
tggtgaccatgcagtcaggg

SEQ ID NO: 176 - DNA, 20 bp, Artificial Sequence
  Description of Artificial Sequence Synthetic oligonucleotide
ttcttttccacttgaggaag

SEQ ID NO: 177 - DNA, 20 bp, Artificial Sequence
  Description of Artificial Sequence Synthetic oligonucleotide
cttcttttccacttgaggaa

SEQ ID NO: 178 - DNA, 20 bp, Artificial Sequence
  Description of Artificial Sequence Synthetic oligonucleotide
gcttcttttccacttgagga

SEQ ID NO: 179 - DNA, 20 bp, Artificial Sequence
  Description of Artificial Sequence Synthetic oligonucleotide
atcaactcccaggatagaat

SEQ ID NO: 180 - DNA, 20 bp, Artificial Sequence
  Description of Artificial Sequence Synthetic oligonucleotide
tccctgactgcatggtcacc

SEQ ID NO: 181 - DNA, 20 bp, Artificial Sequence
  Description of Artificial Sequence Synthetic oligonucleotide
aagctcaagtgtacagttga

SEQ ID NO: 182 - DNA, 20 bp, Artificial Sequence
  Description of Artificial Sequence Synthetic oligonucleotide
ttcacagaggtgcttgctga

SEQ ID NO: 183 - DNA, 20 bp, Artificial Sequence
  Description of Artificial Sequence Synthetic oligonucleotide
tctatcctgggagttgatga

SEQ ID NO: 184 - RNA, 80 nt, Artificial Sequence
  Description of Artificial Sequence Synthetic oligonucleotide
guuuuagagcuagaaauagcaaguuaaaauaaggcuaguccguuaucaacuugaaaaaguggcaccgagucggugcuuuu

SEQ ID NO: 185 - DNA, 45663 bp, Homo sapiens
gtgtgtggacggaattcgggaccgactgacggccggccggcttcccggaactggaaggtgagctccgaggtgggcctggccacccctcctcggttaggtagtacccgagagggcgggggccagtaccggctacttggggcctggggccgagttcgccccgagagaggaaaccgccctccttgggcatccctcctggatctggcgggcccgggagcggccagtctgaagccctgcggctttggcccttcccctcgcgggtttttaggggcgatgttcttgctcctttgcttggttttgggggtagggatataggaacagactgtcctgagaaagccagagtcagcgagtccccggggcgggggctcttggactgcacccgcagccggggcctgcctacggaccttggggccttatttctgaggcctttgtggtgtggggtagaggatgccaggaggccttgccctgaatgtttcagtccagagctaccctctcctttcataccacttactttctgggtttggaaaagggacagctgcctactcggctgggctgggtcgcccgtttctcctttgcctgtcagttcggggggcaggagtgtgtggattgtttacggccttcatacatttcgtgtcgaatttattttctgaagtttctcaagataatccaatttattaaagtttatcccacttgatcatgaggctcagaaatgcatttaggcaccatttggaagttaaatattattaacttgggacttaagaattagtacttaggttggaacgaaggtcaacgcagcatatttgcaataaataaaaaacatttggaaataaagtcgggcattttgttagggagtgagaaattgcacttggttttttcttagtgtggtgcagagtaaaagacaactaaagtagcagtcagaagttttaagttctagttttttacgttttgcatctaagtagatgtctaatttttggcaagttgctgaatctctagctctggggagtttttctgtaaacgaaggggtttacctttagggctatgacaatttttccgaggcactgaactttgagtagaagtgacataagcatttctttgttttggaaagataacttgaatggctgcataaaggatgtattggaaagaggaattttaaacgcggggtgaccagttagaggcctgttttaatagtatagtagaaagttactgagagacctaaacagggcattacagtagggaacagagaacattctgggaacagagaatcacgggaattatttaaaaatggagaatgagagagatttaaagacctagaatttgcagctgagtgggaggaagattgggcgatcattgaaataggtaatacaagaagaacaggtttggtaaggaagataataatctggattttggacattgttaggtttgagatgctgttggatatacatgtggagatacctagcaagcagttagaaatgaaggtttggagttcagaagagatgactagagaaaaatgtaggtatcatttgtagtgtcattcattcattatacaaatatttagtacctaccagttgcttactgggaagcttgtatggaagccctttaagctcatagtaaacacattagaagtgggaagtgaccgttaatgggcacagggtttccttgtgaggtgatgaaatattgtaaaattgatcgtggttctgcttgtacaactctgaatgaactaaagatattttaattgtactctttaaatgagtcaatagtatggtatgtgaacgatatcttagtgaaactaatttttaaaaaagatttacattgcagttttggaaatctgttgagtaaggaattgggaataggaatgtttaattgtgtatttgccgttacacctatagctaatgtacatagtacgttgctctgttaaagatgagatccccacagtacacttgataagatactgagtgctctagtacaaattcttttgagttcagttgcatatcttaatgcatcctgctattttttattttattcataccgtacacatacaaatcataaacttggacacaaaaattgtatggtagtacttagttataatatgaatacccagtgcttgagaaaatgctcgtgttaatttttaaagattattgaagtgaaaacactgaaataaaaagatgtcatagataacataaaaaggacagtaaatagttgtctggttcctcagagattttacaatcctggaatctcaaagttggaggaaccttaaggatgacttaaacaacaaacatctgataatttcttggcttgttaagacaagctggaattggggaagaggaattcttgttttttttgagacagcctccatttgttacccaggctggagtgcagtggcgtgatcactgttcagtgcagcctcgatctcctgggctaaagcaatcctcctactcagcctccctagtagctaggaccacaggcatgtgccaccatgtgatgctaattgaaaaatttttttttgtagaaatagagtctcactggctgagatcgcgccactgcactccagcctgcgtgacagagcgagactcagtctcaaaaaaaaaaaaaaaaaaaaaagaaatagggtctcactgtgttgcctaggctgagaaaaggaattcttaggccatggagattcagggtcaaacctagagagatgtaatcactattcacgccaatgatttctgcatacttctggtgtgggtgggagtcctggctttgcaacttaataacttggtaattaagtaatatactgagccttggttccttcctctctaaatagagggatggataaaccatatgaaactgttgcagtgcttgaataagaaaaatatgtgtggaagaaaagtcagggatttatgatactttatgtgggaaacttggaaggagcatttccatttatatcttgtaaatggttagttttttttttttttttctcgacacagagtcttgctctgtcgcccgggctggagtacagtggagcgatctcggctcactgcaatctccatctcctaggtccaagcgattcccctacctcagcctcctgagtagctgggtctgcaggcacatgccaccacacctagctaattttttgtattttagttgagacggagttgcaccatgttggccaggatggtctcaatctcctgaccttgtgttccgcctgcctcggcctcccaaaatgctgggattacaggcgtgagccactgctcccggcctaaatggttacttttttacaatgaaaaatgaggatttcaggtagcttagaaagagagagggagaaaccaacaggacaaaaaaacatttattcagttacttttttttttttttgagatggagtcttgctctgtcgcccaggctggagtacagtgacgcagtctcagctcactgcaacctccatctctcgggttcaagcgattctcctgccacagcctcccgagtagctgggattacaggcacccgccacatgcccggctaatttttgtatttttagtagagacggggtttcgccatattggccaggctggtcttgaactcctgacctcaggtgacccacccgcctcggcctcccaaagtgctgggtttacaggcgtgagccaccgtgcccagcctaagtgccctctttgtaaagtctgtcttgttctacctttccacactacctggtattaattggaaactttaaagtacccattgattgaaaatatacccatcctggatacattttacactgtatgattatttgtaatagtgctcccaattttagtaacaattgtacaaaataaaatggactcacattccaaaatcctgaaaagaaactaagctataagaaagtaagaatttaagttatagggagtggctgggcgcagtggctcacacctgtaatctcagcactttgggaggccacggcaggcggatcacctgaagttgggagttcgagaccagcctgaccaacatggagaaaccgtctctactaaaaatacaaaattagccaggtgtggtgtctcatgcctgtaatcccagctactccaggagtctgaggcagaattgcttgaacctgggaggcagaggttgtggtgagctgagatcgtgccattgtgctccagcctgggcaacaagagcaaaactccatctctaaataaataaataaaagagagagaacagccgcatcagggtcctgagagcactacctttgggaaacgctcttcaaattttaacttttttttttttaaatgtaattttaaaaattatcaaaacaatatggttgccttttagaaatattggaaaattgagaaaagaaaattaagtcacaccagcctaccacacggtatagctacggtcatagctgtatgaatctccttttactttttctgatgcttattataactagttttaataatagcaagtatataattttgtattttatttttataatttaacatgtcagtcacttgttgttgcattgtcttcatagcatttgtttgttggtttgttttttgagatagagtcttgctctgtcgcccaggctggagtgcagtggtctgaccttggctcactgcaacctccacctcctgggttcaagggattctcctgcctcagtctcccgagtagctgggattacaggtgcctgcgaccatgctcagctaatttttgtatttttagtagagagggggtttcactatgttggccaggctggtctcgaactattgacctcaagtgatccacctgcttcagcctatcaaagtgctgggattacagttgtaagccaccacacccaacccttcacagcatattttgaagtgacagtgccatatttaatcaaccattccctaattgggggtgtgtgtgtgtgtatacattgcttccagttgtttattacataggataagacggacatctttggacatagctctatccatatttaagatagttttttagattattagaggttgaaatactgcatcaaagcgtagtatttttatgggttttgatgcctgttgagcttgttgtattttaaaagaagtgtttcggtttgaattctttatttgcttgcttgttgtgattttttttgtatatgtaaagaagtgtctttatgtacctgtgttttcccactgctcttaatgccccctctctcttttttacccctattctaggttacattgattacccacctagtacaacatcttacgggaagagcatagtatttcctagaggaatatgaacataacaggaaggtatcattggctctgaattaaatttgaacttgtcccctgaatagctacaggttttggaagctgaatcaatgttatcagatgagttagaatccaaaccagaggtgagcccagtcagcttgtttccttttcttttggtgggctatatttcaaagtttatcttcagtttagggggtcagcaaactttttctttaatgggccagataataaatattttaggttttttaagctatatggttttgattgttactattttttcaaagatttaaaaattatattttttgtgattgtttttatatctgtttttcagtatatgtgaaatcatacatttagataatgtaataaaatcttataaataaaaatatatgtttatgtataaatatatacataaatatgagatacatgtgatgtataggtgtataagtatataaatatttactactacaaattcttttgtttattttatatatatctgtaagtattttctttctaaatgtgattggggatttaaacattttttgttgttcacttttcttactttaaatttagcacatctgtgttgcagagtggtcactgaatttggaatcagattacttaaatttgaggattggcattgctcctaactagtcttgtgaccttagacaagtcacttaatgtcttcaattttattcctcagctgtaaaatatgccttaagtttgggtgcggtggctcacgcctgtaatcccagcactttgggaggccgaggcaggtggatcatgaggtcaggagttcaagaccatcctgcctaacatggtgaaaccctgtctctactaaaaatacaaaaaaaattagctgggcgtggtggcgggcacctgtagtcccagctacttgggaggctgaggcaggagaatggcgtgaacccaggaggtggagcttgcagtgagccaagatcatgccactgcactccagcctgggcaacagagcaagactctgtctcaaaaaaaaatacatcttaaatcttactgggttaatgcgataattaaatgagccagtacacagaaaagtgttttgtaaaatgtaatcaatatgatgcagttgattttttttaagccattattttaaaatgtaattatttttatcatttctctgtttgacagaaggtggcagaggttaacttaattacagtgaagtgtcacttagcgatagagatacattctgagaaatatgatggtagttgatatcattattatgcgaacatggtagagtatacttacacaaacctgtatggtttagcctgctacactctaggctgtatggtataccttattgcccctaggctgcaaacctatacagcatgttactgtactgaatactgtaggcagttgaagcacagtggtaaacattgtgtatctaaacatatctaaacgtggaaaaagtacaataaaaataggatattacaatctcacaggaccatcatcctatatgtggtctgttaactgaaatgtcgttttgcagcacatgactttatataaatcagctgttctctagaacagagaacttgataaaataactttagatattatgttgtttagtttttgtttgaatatggatgagactagtgaacagtcttaatattatcttttggtaaattcaaaggtttagatacatttctaacaactcgtatttctcagtgttgtcttttctccccccagctcctggtacagtttgttcagaatacgtccatcccattgggacaggggcttgtagaatcagaagctaaagatattacttgcttgtccctccttcccgtgactgaagcctcagaatgcagtcggctaatgttaccaggtaaaaattaagatcactagaaataggataaagaagtcttaagagcttagcctactctatgttactggtggaatgaatatatcgtttaataactgactggtggaaactgtaaaaattaggagtcatggagaaaaagcaaccatgaaatcattagtgcattggtagtttaagccataggaatggatatgatcacccagagagtgtgagagtggggaaaaaaggtcagtgcaaaactatggaactctttatttaaggtacagggtagggcaggagaacttgtaaaggagggaagaaacaaagactaaaatagttggatgaacatcagcagagtggaggcatgaaacccaggagaaatcattgtatcaaatgctactgacaggcccagtgaaatagggactaaaaggactccattattgactttggcagaacctgttccatggagagaagaggatggaaagtcaggctgcagtgacttgagtatacatacttgcttttttttttttttttgagatggagtgtcactctgtcgcccaggctggagtgtagtgacgcaatcttggctcactgcaacctccgcctcccctcccaggttcaagctattctcctgcctcagcctcctaagtagctgggattacaggtgcgtgccactatgcctggctaatttttgcatttttagtagaaacggggtttcaccatgttggccaggctggtcttgaactcctgacctccagtgatcagtccgccttggcctcccaaagtgctgagattacaggcgtgagccactgcgccaggcctatcaatacatactttctaaagtctctcctggtacgagaaggaaaaagagaaggcggtaagatagagggaaattttttgtttgtgttaagatctgagatatttgagggttgctttatgctaaggggaaagaaccagttagaaaagttgaagacaggccaggtgtggtggctcgtgcctgtaatcccagcacattgggaggctgaggcaggaggatagcttgagcccaggagattgagaccagcctgggcaacatggcgagacctcgtctctacaaaaaattaaaaattagctgggcatggtggtgtgcagctgtagtcccagctgaggtgggaagatcacttgagcccaggagtttgaggctgcagtaagctgtgatcacaccactgtactccatcctgggtggatagagcaacaccctgtctaaaagaaatagtaataaatttaaaaaatatatgtataaacaaacaataattaaggaaatgaaaacaacagcgttcatcttcccttagaatgactgttatcttaaagatggaagataacaagtgttggtaaggatatggagaaaagggaacctttgtacactgttggtgggaatgtaagttagtacagccattatggaaaccgtatggaagttcctcagaaaactaaaaatggagccactatatgacccagcaatctcacttgtaggtatatatccaaaggaattgaaatcagtaagtcaaagatgtctatacttacatgttaattgcagcattattcacaataaccaagatatggaatcagctttagtgttcatcaactaatgaatgcatttaaaaaaaaatgcattcaccttcttttccacatttcttttagttttaattttgatttaaagtattttagacatacaagtatagataaaagtaaatgaaaatccaaatacttggcctcccagctcataaaacacaatattacaggccgggcgtggtggctcatgcctataatcccagcactttgggaggccgaggtgggtggatcacttaaggtcaggagtttgagaccagcctggctggtgaaaccctctctactaaaaatgcaaaagttaaccaggtgtggtggcacagaaaaaataaccgcagcctgagatacaaccataggtaaaaaggtaatggcaaaaactgcaatagcttttgcaccaacctaatacaaaagttaaggagagggagctctctccaggaaaaataaaatgtagatcctattcttgagttagtattcattgtatagctatatttttcaaggagaaagtactgatttctggcctcaaaaaggactaaattctgtgatgtccaaatgttgaatggttggttattcaccaaccattactgccaagtgacagagaattctgttcctcacgttttggctgactgcctttggaacataaagccatctctctgttcttctgtgagacaaagtttttatttgtgtaatgagaatgctgtctgttgctataaggatgttagcttccaagagatgttgggtgctgtgtatcaaaatggtgggctcctagagataggtgctacataaagctgtggtattttccctttactagatgatactacaaatcattctaactcctccaaggaggtcccttcctcagctgttttgagaagccttcgggtgaatgtgggtccagacggagaggagacgagagctcagactgtacagaaatccccggagtttttgtccacttcagagtcttctagcttgttgcaagatctacagccaagtgatagcacttcttttattcttcttaacctaacaagagcaggtattctttccttttttttgggctctgcttatgcctaagaaaaaaattaggggtggggcatatttaacttagtcatacttaaaatagaaagaacctttattattttattattttggagaattggtgtgtctggtgaccatttgaatatgtagagggagagggaggactctagtatgactcatatcttgtttgggaaattatgtagtgccatttactgagatcgcacaggaagagcaaggcttgggggaggatgattagttccaatttgcacctatttatgttgagattttggaggtatattcaggaggacatgtcctttagagagttagaaacatgaatccagagttctagaaaggtctgttttggtgcttatcagcattcaggaaacgtatgaaacaatgaacatggattctagagaagtctcaagcataaacagaaaagaaggcaagtctgggaccctgggaaaaaacatttaagaggacaacagaacaggaaggagcagctggaaggggatgaagagaactgggagagagtggtagcagggaagccaagggaggaagaagttttaaggcagaaaagatactaaatcatgccaaatactacatgatgggcttattagtgagaagaagtgtgtgtctggtgaccctagcaagagcaattgttactgtggtagctgaggtagaggccagggttaaagagatggggagtggatgggaggtaagacaatagatacacaagttagaaggtacacttgttcaagaagcttgactttaaaggaacagggaagaaggtaaatagctagagaagtacttggggttttctaattctttttaaaaataagggtgagagacttgagttaggacatatttaaagacataaggaaatgctggccgatagagaggaactagttgcagataggagagaggagataacacaagtatatatggggggataattgaagaatgagaatgaaatacagtaggtggggattagtctggttgaccaagaaagacagctttgccttgaaaccagaggaggcaaggtaatgacaagcctgtagactaaaggtagaaagatgatagcctgctcttcagtgagaagtttgaggtaaaaggcgaaggtcaggaatagcttatttaggacatggagagtttctgggccaggcgcagtggctcacgcctgtaatcccagtactttgggaggccgacgtgggcgaatcacgaggtcaggagttcaagaccagcctgaccaacatggtgaaaccccgtctctactaaaaatacaaaaattagccgggcgtggtggtgcatgcctgtaatcccagctactcaggaggctgaggcaggagaatcacttgaacctgggaagcggaggttgcagtaagccgagattgcgccactgcactccagcctgggtgacagagcgagacgccgtctcaaaaaaaaaaaaaaaaaaagggacatggagagtctctgagccagggcaaataaaaagcttgcttggtttctaacttttcatatattttgtttcttccaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggtgaggcccgtcggcattttccttgccactattctgtgcaaagagattttggtaatgcagtggctggtttgagttgtatataatttaaattattctattgatgagagcagaaggctagaaatctgtaaattgtttaaattttattttctataaaataacttggtcatgccagatttgtgttcaaaaccaactagtattctgaattctcaactttaaataatgtacagagccccttttaaagaaaaaataatatacatcctggggtttgaattgtttttattgtaatgtctcttaaatatgcaaaaacataaattctgtgtaaacttcatatacttagagctcttataccatcatgaaatcaaaacagtttttatcagttaataggaataaaactaccaaatccatgaaaataaaaataacattaagtcattgtggcagaggattatgttttgctgaaactaaattgctgctcatggcagtttaacaattagctatgatggttttaatgctttttcttttcagttcagcatgcctatactacttactctgtgtcacttttctctgttctagtaattttcaaagcattgtcactggatcattgacgtcacgtgggaactgagaaatataaactctcaggcccatgccagacctactgagtcaaaaacttggttctgatacatgttaaagcttgagaaccacggctctattcagaccactgcagaatctttgcttatgtagcatgtcatggatacttttttaggtctgcctctgtgttttctcattaacatgtgacacaactcaatacaacatggtttaatgctatcattatcacaaactaagcttaagcacttagtacacagttgtttggagtttatgcagattatctagtatatgcttattattattttttgattaccattcaaatgaaaataaaaagttaaaatttctcatagagaactagagttccctatgaatattttgaaacattaaatcttttttttttttggtctttttttttccctttttatggagaatgaggttttgctctgttgccagacaggtctcaaactcctggactcagggtctcctcctgcctctggctccctaagtgctgggattttaggtgtgagcaaccatccccagccaaactctaaactgtttgttttcaaaatacttagttctgtttatgactaccatagtgctacttagaatggttcattttaacattttggtgggacccttaaagttcagattcttcctgagatatatgtgccaatctccagatgctgaaaccttcagcatttcctcttacattgaagaaatagtgtcatatttgccaatgagaagcagcgtagggaggggagcttgcaatttagaatctgggagccctgggatccaggcctggcaccatcatttactggttccctgccttgggcagtttaacctcccttagcttcaattttcttacgatctgaacatagagatcaggcagctttcctgactggcttgttatggagattatgtaacattctatctgggaaatgttataatcagcaaagcattgtgtggatgtttttcttactctcaaagtatgttgccttactatcttaacaactactttgtatgcctgaatataaaatcaccccaaataggctgggcattgtggctcatgcctgtaatccctgcactttgggaggccgaggcaggcagatcatctgaggtcaggaattcgagaccagcctgaccaacgtggagaaatcccgtctctactaaaaatacaaaattagctgggcgtggtggcgcatgcatgtaatcccagctactcaggaaggctgagacagaactgcttgaacccgggaggtggaggttgcagtgagtggagatcgcaccactgcactccagcctgggcaacaagagcgaaacttggtctcccaaaaaaaaaaaaaatcaccccaaatataagacaatcttttattttccaacaagataataccctaaaagccttttatttttttctaacttagttatgtaaacttttaagcatgtagataaaattaacattataggttgctatttggaagctttatgattttggttatacatacacattttaattttttttttttttttgagaccgagtctcagtctgtcgcccaggctggagtgcagcggcgcaatctcggctcactgcaaactctgcctcctgggctcaagtgattctctgcctcagcctcccgagtagctgggactacatgtgcatgccaccatgcccagctaatttttgtatttctagtagacacagggtttcaccgtgttggccatggctggtctcaaactcctgacccaaggtgatctgcctgcctctgcctcccgcagtgctgggattacatgcgtgagccaccgtgcgtggtcccacatatacattttaaatcatatgaaatatatcaatattgctttcacccactaacacaagcttgtccaacatacggcctgtgggcacgtgtggcccaggatacctttgaatgcagcccaacacaaattcgtaaactttcttaaaacattatgagatttttttgtttgtttgcttttaaagctcaccagctgtcattagtattagtatattttatgtgtggtccaagacatttcttctttcagtgtggctcagggaagccaaaagattggacacccttgcactaacatgtaatgaagtgattttattcaatattttaatgcatttttaacataaatatttttgtcattgttgacactgaagtcactttatgagtcctctagcatagtttttcagagcatgttatcttcacttacagtctaagttgtttgaaatacagaaacgttttaattttgcataagatgttgtatatcagatcagcaaaggcctttgggccaaatccccacctgtttttataattaaagttttatgggaacataggcatccacattacttacaaattgtctaaggctgctactatggcaaagctgagtagttgtaacagaaaccatacggcagccgggcgcagtggttcacacctataatcccagcactttgggaggccaaggtgggcagatcactggaggtcaggagttcaagaccagcctggccaacatgatagaactcaaatacactcttttctgagggatttttttttttttttagggaaaaatttgcgttatatttatttgaccatatattgataatcttcattcttgagcaggaggagtttcagtgatttcttagccttgccttattctatgtattcatttttcctattttatgattatattaatattcttattgtatctataaaacggaatccagaaagcccctactatcactgatttgaatcactgtattagcatgatttttttggccggtgcagtggctcatgcctgtaatcccagcactttgggtggccgaggcgggtggatctgttgagctcaggagaccagcctgggcaacatggcaaaaccccatctctacaaaaaaaaaatagaaaaaattagccaggtgtggtagtgcacacctgtctctagtcccagctacttgggaggctgaggtgggaggatctcctgagttggggaggtcgaggctgcagtgaaccgagatcatgccactggattccagcctgggcaacaaaatgagaccctgtctcaaaaaaaaaaaaagaaaaaatttaaaagaaaaaagcatgatgttaacagacctttcattagaaatcaattagctggagttctggggttggtgggaagattccttcacggcatatgtatttttgtgctatatgtgtagttttttttttttttttgaaacggagtctcgctctgtcgcccaggctggagtgcagtggcgcgatctcggctcactgcaagctccacctcctgggttcacgccattctcctgcctcagcctcccgagtagctgggactacaggctcctgccaccatgcctggctaatttttttttttgtatttttagtagagacggggtttcactgtgttagccgggatggtctggatctcctgacctcatgatccacccacctcggcctcccaaagtgttgggattacaggcgtgagccactgcgcctggccctgatatttttcaagtaaacacatttttaaaaaccttggggcaatattaaagaaaaacttaatagtaactagcaggaagcatcctaggttctgctctgaccttggagcaattagttttatgactagtatttggtaaagtatagcaaggagaagattaatagatgggacagaaactgcctttgattaaccatcaggttctaggggctgtgataggcacaacatatatattctacttttggctattgagggggaaatttggagatacaaatcttattgctaaggtatatatagtggaaagatacactgcctgcatatgctcatggcagaattaaatgactattaatttttttctctattgatgttcaagtatttcttgaagaaagtgaaagaaaaggaccaccacctgctaatctggaatttatataaaaatttttaagtaactgattttgtttacatttaagggaggcactttgtactcctctcctcacctctctttattctgttgaacaaaactccagtagatattttctcttactttttaaaatgtggttagaaatttaaaaatcatctgagtttttgaattccttaagtattttataactaaagaagggtggtacaggaaattggaaggtggttcttagtttgcagaatgacaagaaatagccaaattcctttcagaagatggaaatctaaaaatggctgtcagagcccacactgaaccagaatgactacttcttcttaaaatacagtgggattttttgtttgtttgtttttgaaacagggtcttactctgttgcccagactggagtgcagtggtgcaatctcggctcactgcagcctccgcctcccggacacaagcaaaatacagagtttttattactcagaactttgtcatctttgtagcaccctactcagggatagtgttgctatccctgcttctccaccaaactgtgtaatgcaaaatggttttaattctgatttctaatagcaaaccattttggatatctcttacatttgtcaaagaggcctagtcttgtgaaacaaagcatgatagtttttggtagtttttctggagtaagggaatgaggatgtagaacacttgcatttctattgtttcattatatttatttattttttgacagattcttgctctgtcacccaggctggagtgcaatggtgcagtcacagctcactatagcctcaacctcccaggcttaagtgatcttcctgcctcagccttggagcctcaactacaggactgcgccaccatgcctgtctaatttttgtttgtttgtttgtttgtgttttgtaaagatatggtttcaccatgttgcccatgctagtctcggaacacctgggctcaagcagtcctcccaccttggcttcccaaagtgcttgggattataggtgtgagcaccatgcctggccaagtttctgctgttaagtgcatacaaagataactggggaagatgaagatagatacctgattaatgatcactaggagtaaaatataggagctttattgtttgagcctaagtatctgagtagggtctttccctggattacaaatatgtgactctgttacaggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcggtgagcaaatccgagatctcatatttggatatacgttgatgcaaaagttgattcttccagaggaacttgcccctcaggttgacaaattacaggaaagtagggactccagttaacttctggttaagatatctttttttctttaatagaaggatttgcattgtctcatttatctcattcttgataaatatttaataagacatttgggacctaaaggccttaaaccaagagttctcaaattttattataaaacatatttcctttgagaacttaaaagtaacaaataagcatgggctcctaccttgaacctatgagatcagtttaaggggtggtgctaggcatctgtagttttttgtttgattttttttgtttaagagacaggatctcactttgtcacccagggtggagtacagtggcgccatcatagctcactgcagcctcgaactcctgtgctcaagtcatcttcctacctcagcctcacttgggattacaggcttgcaccaccacacctggctaattaaaaaaacattttttaaagacagggtctcgctatgttgcccaagctgatctcaaactcctgggctcaagtaatcttcctgcctcagcctccagagtagctgggattacaggcgtgagccaccacccagctatagttttaacaaagtcttcagttgattctgatatatccagtttttggacttactgtttgccttgagagtcaataggggctttgacttgtcatctctagcaaagagtgttttcagatatttttccttatgtgattatttttgcagaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggtgtgtgcagcaccaacattcctttgggcagtctgctccttggctctgcatgtttgcctctccttaggaaagactcaagtgggaatagaaatgaaggtctggggttgttgctagaactctgaagatgggatttctttttcaaatgacttgttgcacttataataacatttattgtatttttacctgtgtgtgtagagtagacagtgttgatttctgttttcaggagcctaacctgcagggagagctgtaagaccttctcagacaacaatgatgtggggagatgtggacaaaatcagtctgtgttcaaatctttctgaacactgcgcagttatgattttgggcagtaacatttactgagtcacacaaaccatacagaacctgttctaggcactaaatggggcagggtgggaaaaaaatacaaaagataagatataatccctgactttcttgttagagcctcacaacgtgagaaacagaatagagaagtgaattattctgggtcacacagtatgttaatatcacaggagcaaaataaagattccttgactcttaatctaagagtctaagggtgattttccaagccacttggtccagcgtgagctagtctctagccaaagtataatcactgaacccacaccactcttagaatgtgtaactattcttccccacttacttgccactttgactcttcttgattcttgcattggcaaaatgacagcaggtaaaattaaaaggcatatatatggcatatataaaatgcatatataaaaatataaggcatatataaaatataaaaataaaaggcatatatatacatacatatatacacatacatatatatacacatacatacatatatatacacacacacacacacacacacacatatatatatatatatatatatatatacacacatactttttttttttttgagatggagtcttgctctgttgccaggctggagtgcagtggcgagatctcggctaactgcaagctccacctcctgggttcatgccattctcctgcctcagcctcctgagtagctgggactacaggcacccgccaccaggcctagctaattttttttgtttgtatttttagtagagacagagtttcaccgtgttagccaggatggtctcgatctcctgacctcgtgatctgccctcctcagcctcccaaagtgctgggattacaggcttgagccactgtgcccagcctatactttttttttttaattagtcaaatgcagtagtgagaagaggggaaaaagtaaaacaaggcacttgatctgtaactgactgtgaacatcagttgagataactcactaccttcagaccagcccattgttgtagcatttgatacctctgaacatcatcttaaaatgctttcttttttcttatttatttatttttgagacggagtctcactctgtcgcccaggctggagtgcagtgatgcgatctcagctcactgcaacctctgcctcctgggttcaagcttttctcatgcctcagcttcccagtagctgggattacaggtgtgcaccaccacgcccagacattgttgtatttcttgtagagaccgggttttgccatgttggccaggctgttctcgaattcctgacctcaagtgatccgccgaccttgccctcccaaagtgctgagattatatatgtgaaccaccacgcccagacttaaaatgcttttacttcaaaatatgacctgattttcccgttgtttttttttctctaattctttctagcttttatctctgttaatctctgaatgtttcaccagtttcagaatttcttgttccttctcttttactcagtgtactctcttgtcataatctcaggcctctatttaattatttctccctagatgcctcacagatctgaatctcaataataattaatataataataaaagttagcatttgttgaggcactttagtatgtaccaaccacatatacattatctcatttaatctttaaggtaactttatgcattagatactgtacttattgataaaggaacagaagattagagagagaagtaacttgcccaacatcatgcaggtaggaaatggcagagctggaatttgaattctgatatgaccccaaagctcttccaaggattttatttttaaatgctgacaactcagtttatctcaaatgtcaaaattgacctcaaagctttattagtagtaatgatagtaaagctgcagttcaggtcgggcccagtggctcacacctgtaatcccagcactttgggaggctgaggtgagcacatcacttgagggcaagagttcgagaccagcttggccaacacggtgaaaccccatctccactaaaaatacagttagccaggcatggtgatgggtgcctgtaatcccagttactcgggaggctgaggcaggagaagtgcttgaactcgggagttagaggttgcagtgagccgagattgcgccactacactccatcctaggagacagagcgagactccatctcaaaaaaaaaaattttgactgggtgcaatggctcatgcctgtaatcccagcactttgggaggccgaggcgagtggatcacctgaggtcaggagttccagaccagcctgaccaacatggtgaaatcccatctctactaaaaatacaaaaattagctggggcatggtggcacatgcctgtaatcccagctactagggaggctgaggcaggagaatcgcttgaacccaggagacagaggatgcagtgagtggagattgtgccattgtactccagactgggcaacaagagcgaaactctgtcttgaaaaaaaaaaaaaaaagtaaatctgttaattcagtggcttactatgagggcttactatgtgccaggcactatgctgaatatttacctggtattatttaatcctcaaaacactcttttgaaattgtattatctccatattgtagatgaaaagctgatttttagagagattaaacaccttccccatgcaaataagtggtagtgccaggatctgaagtcagaattggattaacagaccccaaaggccatgtttttcaccattttgctgcattgtcttttaaacctcttccttctcctgtgtccgttatcccagtgaaataaccatcctctcaagtttccaaataggcaggtatgtcccctaaccagcttgatgcagagatttcatgccattacattcttatgtagaatctttttctaaatcctttttgttttttcagttctttagttcatcttttgaaacatttgatcttgttattactaaaattttttttttacatttgttctgttatatatattagtatattgctcattctttcaataactttttgagtacctagcattctgagggatgagtatacaattaaaacattgaaaaaagctaccggtggagagtctcaaacattaaactgtgtggtacagtctaaaagaatagtataatctagcagtgctgttccctcccagaaagctcagctcccatattgtcactgtgccagccttactagccattttctgaactttccccgggcactctgtttcaaggctctgctcacagtattcctggtgcctgaaatttccttccatccttgtcatcttttagatgaactcagaaattcagctgcttacaaaaattactgcaacggagcacttttttttgtatttatgaattcatttgtctgtctcacactagtttgtgacccttggaggatagagactgcatcttttttgtttgtttgtttttttgtttgagacagggtctctctctctgtagcccaggcttgagcccaggaatttgagatcagcctgggaaacatagtgagacttcctctccaaaaaaattatacttttagatggatctacaaactgagtcataaagaaaattgatgtaaggtaaaaatcctgacagcatatacagtattaatcttgatatcctcagatggtgggagtcactgaagaaggtaggttaatgcaactctccagctcttgggccaggagggagaaggaaatataggcttacattccaaggaaaagggcaaggctgtctggtggcatatatactaaaagatcattgagaggcaacacttgctaggtagcccagttgaaattataacatgaattaaataaatcagagtaggtaagtcattagactctgaataaagttggtgggaaaagagagtaacaggtaatcagttcatcagaagactgagtctttatctcattttctgtctctaggagagaaacctttcctttgtgaagcccaaggatgtggccgttcctttgctgagtattctagcctccgaaaacatctggtggttcactcaggtatcagaacctctgccattcatagattttaggaaaagtagacaaaagaataaaagggttgtgggggtgatataaagacaactaaaaagaaacaaactgttcactataaagatagaaagcttctcatcatcatgacagaatcagaaatcaaggcccttaattcttataatcaaggttgggggggggacatctaattattattttcagctgtgtttctaataaagccttcttttgaagagatcagcaaatttgtttggcactctgctgggtgctttataagttacataaaacatggaccctcttctttaggaattgacatttttatccttggggaggatttcatatcagaagaaagtaaaaagactgatataagttgtagcagcaccagtaatattactgccttctgagcaggtgttagtgatcctgcttgagtcaccattttggcagaaactatgcctttggcaaacagaaccacaaaccttatcattgcagatgtggagaagagaaaagaggagtaagatttagtgacccattttctgtttggagccttaaattaattccctgcaattctgtcagtttgctcactctgtgcagatatatctcatcctctcaaccagaagtcaggtacagcgcagggaaaagcctgtatcttgttagaaggacttttttcctgtaggacatggtgatttacttggaaaacaagatggggaaggtagtttaagaagggcctaaaatgccaggaaggagagtttgtggagcctactggaggtacaacctgatgagaacttggcttactgaagatttatctggcagtgatgcagaagctaaactaaaacaggaagagatgagaagactttttttcagtctggtcaagaagtctgctccagtgtagtggcagggagcatggaaagagacccagtggaagaatctgttcaactccacaaatgattggctggctataaagttatacagcaatggaggcagcaggtgaggattattgatgaataatcaataatcagaagcatggtagtggcctggcgtggtggctcacgcctgtaatcccaacactttgggaggccgaggcaggtggatcacttgaggtcaggagttcgagaccagcctgaccaacatggtgaaaccctgctgagattagcctactaaaaaaataaaaaattatccgggcatggtggcacatgcctgtaatcccagctacttgggaggctgaggcaggagaactgcttgaacctgggaggcagaggttgcaatgagctgagattgcaccgttgcactccagcctgggcaacaagagcgaaactccgtctcaaaaaaaaaaaaaaaaaaaagcatggtagtttcaggaggttgaggctgcagtgagttgtgattgcgccactgcactgcagcctgggtgacagggcgagaccctgtctcaaaaaaaaaaaaaaaaaagtggtagtgaagataaggagggaaagggggctttccagttgtcaggctgactcacatatttataagaggggttagaaaaatgttggatgatgagtgctagaggggctctgtagactagccttgtaagtttgttggaggtgagaaaattcctggcacacaacagctttttagaatttgttgattgaaccaattagaatgaataaaaggattgctaagtaacagtgaatgaggacatagctgagagtaagtagcataactttttttagtagacctgttcagattttattacttttctctgctactgcttaacagtttaaaagctgggtactggagaaaataggtgatggaggttaatcagagcagaggcttgctgaggaagagatgaactcaaggaggagagggattctagaatggaatcaggtcattgttgaatgactggccgtgggaaacaaggggaagccagaacatgacaagtggagaggaggaactggaaagttacttatgaggaaagcaagctactgcagcaggagcaaggaagttggagagttataaacaatcagggatgtacatttcagagttgagatcttagaagtagagcagttttttaatatgatgacaagttctaagcagaggtggcaaactggtatcctgtaggctagatgtggacttcagatgtgttttgcttggattgtagagcgtttttattttactttactgtgtcagattatgtgaaagattcaccaaagtagagaaatttttttttatatagcaacagtggtgttatacctgtcaaaattaatatttccttggttttatcttataaccaatccatgttcagttttttctacttgtttcgaaaatgtcttttaaagttggtttgtttaaatcaggatccaacaagatccaatattatatttggttattaagtctttaaaaatctagatctttctacattgagttgttgttgttgttgttgtttgtttgtttttttcatagagttgttgcaaaacagagtcagttgactcctatagaatagcccacattctggatatattggtttgcttccatttatgtcatttaacttgtttctctacgtttcctataaatgtaagtaagctctaaaagcttcaaagtttggaagttttgggcaagaatatgtcagagatggttctgtgtgcttcgttttgtgtcacatcaggaagctcacagtgcttggttgtcaaaattgatccatgggttcagctggcctgatccctgtattataaggttccctatgaacctttcactttcttttttttttttttttttttgagacagtattactctgttgtccaggcgagagtgcagtggcgtgatcttggctcactgcagcctccgcctcctgggttcaagcaattctcccgtctcagccacccgagtagctggtattacaggcgcgtgccaccacgcccgactaatttttgtatttttactagagacggtttcaccatgtggccaggctggtcttgaactcctgacctcaagtgctctgcctgccttggcctcccgaagtgctgggattacaggcacgtgccaccacacccagccaacctttcaccttttgatttcattcctaaatgatacctgaatctcttatttcataaaggattgtaaaatggtggtttttcttattcctttactcctttcacattagttggaattcttctgtactgtgcgactctttagttggactcttccactaatatcaagtagggctatttgatcatttgcataggcaagacaggataaatatgtaattcttttaattgtgaattttgagaataaggaattgatgctctagttacttctagtatttcatgtttgatgctatttcaaatgaaatgttttcttgattttatttttgagttgttcattggtagtatatagaaatacaattgaattttgtacattgatcttgcaaccttggtaaatgcttgagttttagtagtttcttttttttttttcttccagctttttagttcaggggtacatgtgtaggatgtgcaggtttgttacctaggtaaaggtgtcccatggtggtttactgcacagatcatcccatcacctagatattaagcccagcatccattagctattcatcctgatgttctccccctcctcctacaccccatttttttttttcttttctgtttatttttttagagacaaagtctcactctattgcccaggctagagtgcagtggcaccatcatagctcactgaagcctttgaactcctgggaacaagtgatcttcccacctcagcctccccagtagctgggactgaggcagaaatttaaaaataaatatgcattcattcactccaagaaaagtaacaggcaaggcaagggttaaaaagaaaagaacaagttttcctctgcctagcaagctcacttcaaggacagttataagataacgctgtttgagaagccaaggccaaaggaatggactccagacaccccctcttccagagcaaggttgaaggaaaaaaaaaggaaagacaaattcctttactgttactcctttccctggcttcttaagcataactgtgttttgcaaatgtctgtatttagccagttcttatttttctttcaatgcagctataaggtcaccagctatgcaaggccacaagttatgctatgctataaagtatgtgacctgtcatgattaactgcttttgttttacttttgtaagtccgcttacaaaaaccccactccgtctttgtttaatgctcagctttttggatgtgaatccactgagctggtgcgtacctaaaataaacaatcctcctgttctccgtatcagtctctccagtcctcagtttcctgcaataggactacaggctcacaccaccacatctagctaatttctattattttgtagaaatggagtctttgtgttgcctgggctcgtcctgaactcctggcctcaagtggtattcccaccttggcctcccaaaatgttgttgggattacaggcatgagccaccacacttggctgagttttagtagtttttaaaaaatagatttcttagtgttttctacataaaagatcatgtcatctgagagtaaagatagttttgcttctgcctttccaagctagatgcctttaatttcctttctttcttgcttttttttgagacatggtctccctctgttgctcaggttggagtgcagtggcatgatcatggttcactgcagccttgacctgggcttgggcagtccttccacatcagtctcccgaggctgggactacaggcacatgcccagctttttttttgagtctcgctgtgttgcccaggctggaatacagtggcgcaatctcagctcactgcaacctccacctcccaggttcaagtgattctcctggctcagcctcccaagtagctgggattacaggcatacaccaccacacccagctaatttttgtatttttggtagggacgggttttcaccatgttggccaggctagtcttgaactcctgacctcaagtgatctgcccgccttggcctcccaaagtgctgggattacaggcatgagccaccgtgcctggcccccagctagttttttcaaaaaattgttatagagacagggtcttgccatattgcccaggctggtttcacactcctgggatcaagcagtcttcctgtctcagccttccaatgtcgggatgacaggcatgagccactgcacctggccttaatttcctttttttttttttttttttggagactgagtctcccactgtcacccaggctggagtgcaatggcgtaatctgggctcactgcagcctctgcctcccgggttcaagagattctattgcctcagcctcctgagaagctgggattacacgcgtgagccaccatacccggataattttgtatttttagtagagacagtgtttcagcatgttggccaggctggtcttgaactcctgatctcaggtaatccacctgccttggcctcccaaagtgctgagattacaggcatgagccacagtgcccggcctactttctttttctggctagaaccttgagtacaatgttgaatagaagtggtgaggccgggcgcagtggctcacacctgcaatcccagcactttgggaggccgagtcaggcagatcacctgaggccaggagttctagaccagcctggccaacgtggtgaaaccccatctctactaaaaatacaaaattatccaggtgtggtagctcatgactgtaatcccagctattcaggtggctgaggcatgagaatcgcttaaacccaggaggtggaggttgcagtgagtcgagatcgcgccactgcactctagcctgggtgatagagcgagaatctgtctcaaaaaaaaaaaaagtggtgagagtacgtatccttgtctttttcccagtcttaggggaaaagcatgtattctttcatgattaattttttttttttaagacaaagtcttactctgttgcccaggctggagtgcagtggtgtgatcttggctcactgcaacctctgcctcctgagttcaagcgattctcctgcctcagccttccgagtagtggggattataggcgctcgccaccatgcctggctaatttttgttttttttagtagagatggggtttcaccatgttggtcaggctggttccgaacttctgacctgaaatgatccacctgcctcagcctcccaaagtgctgggattacaggtgtgagccactgcacctggcctttcatgattaaatttgatgttagctgtagttttttcatagatgccctttatcagatgaaggaatttaccttctcttcctagtatgttgagaatttttattatgaatgggtattggatcttgtcaaacaccttcgtgtgtctgttgagatgatcttgtttttgtactgtatgctactaattttactgctagtagttggtttttggatgtaaaaccaactttgtgtttctagggtaaatcccatttggtcagggtgtatagtcctatttatgtggtgctagacttggttttgtcataatttgttaagaatttttacagctggctggatgtggtggctcatgcctgtaatctcagcactttgggaggccaaggcaggaggattgcttgggcctaggagttctggaccagcctgggcagcatagggagacctgtcctctacaaaaaataataaatcagtaagagagagggagggagggaaggaaggaaggatgagaggaagggagagatagagaaggagggcgggagagtggaaaggaaggaggaaaaggagtctggatgttcatgagcgatattggtctgtaatgttttcttgtgatatctttagcttttctatgagggtaattatactggcctccctggttcaagtgattctcatgccacagtttcttgagcagctggcactacaggcgtccaccaccaatgtcttgttaatgtttgcatattttttagtagagatggggttttgccttgttaggctggtcttgaactcttggcctccagtgattcacttgccttggcttcccaaagtactgggattataggtacgagccaccgtgcctggcctggttgttatttttggagcttctcatgctgtatttatgcttttgacatgacccatttgtcttgttatagcctctctgctttctaacacaacaacagttcccaggcttaccttggacttggaatcagctattcctccatggagtcctggttccttatagttggaaccacagtctgggtgccatgtgcccgttgctgttaggttgtcatcgttggcagggcgcagtggtttatgcctgtaatcttggcactttgggaggctgaggcgagcagatcactttagcccacgagttcgagactagcctgggcaacacagtgaagccccgtttctacaaaaaaaaaaaaatacaaaaattagctgggtgtggtggcatacacctgtaatcccagctacttgaaaggctgaggcatgaggatctctcgaacccaggaggtggaggctgcagtgagccgagatgcaccactgcactccagcctgggaaacagagcgagactgcgtctcaaaaaaataaataaataaaaaggttgttactgtttctagaacttttcggtggaaagaactaaagacatatttttgaaaagaaaataattggttcatgttgataattccaatttaaattttcacattaaacaattttcaggggatttttttgtactttatttttttcttacatcgaaaatcttggttcataacattaatacaatattgtttcaaaatgataataccaatattatcaatttgtctacagaatgaagtttaagacttttggagctgtggttcttagaataatactatgtttaaataccctttaaatcattgttttaatatatcaccagcttgatatgtagatttgcttcagtttctttttcaaaaagccttgattataggtgtgtctctttaccatgttccctccctccttctcttataggtataaacactttttatttcctatttggtttacttttccagtggttctttttgcatatacaagcaaatgcaggttatttaagttgagatatagttcacataccataagattcatctcaaaaaagtgtgcagtccggccgggcatggtgactcatgcctgtaatcccagcattttgggaggccgaggtgggtggatcacctgaggtcaggagttcaaaaccagcctggccaacatggtgaaaccccatctctactaaaaatacaaaaattagctgggtgtggtggcatgtgcctgtaatcccaactactcaggaggctgaggtgcaagaatcacttgaacctgggagggggaggttgcagtgaaccgagatcatgccactgcactccagcctgggtgacagagtgagactccatctaaaatataaaaagaagaaaagggtgcaactaagtgatttttagtagtaatataattatcccttgttatctgtggtgtataggttccaggaccccacagataccaaaatccacaaatactcaagtctcttacataaaatggcatggtatttgcatataacctacacatatcctcccatatgctttaaatcatctctagattactgatagtacctaatacaacgtaaatgctatgtgaatagttatgctatattggttatttgtattttttattgctatattgttattttttatttatttatttttttagatggagtctcgctctgttgcccaggctggagtgcagtggtccacgatcttggctcaatgaaacttccacctcctgggttcaagtgatgctcctgcctcagcctcccaagtagctgagattacaggcatgtgcctggctaatttttatatttttagtagagacagggtttcaccatgttggccaggtcttggaactcctgacctcaagtgatctacctgccttggcctcccaaagtactaggattacaggcatgagccactgcgcctggcctgttattttttatttttattttttctgaatattttttatctgcttggttgaatcaatggatgtggatggccaactatatattcatagttttacagttttctctactgtccgactccacaacatttcatcatcccaaaaaggatccccatatccaggagatgattaaaaaaagaaaaagaagtcccattattagtatcattgaccatgtctctctttctccagcccctggcaaccattaatctgctttctgtgtctggatttgcctgttctggacatttcatatggccttttttttttttttttttttttttttttcccctaaacggagtcttgctgtctcccaggctggagtgcagtggcgtgatgtcggctcactgcaagctctgcctcctcggttcacgccattcttctgcctcagcgtaccgagtagctgggactacaggcgcccgccaccacgcccggctacctttttgtatttttagtagagatggggtttcaccgtgttagccaggatggtcttgatctcctgacctcgtgatcctcccgcctcagcctcccaaagtgcctggattacaggagtaagccaccgtgcccggccttttttttttttttttttttttttttttttgacagagtctcactctggctcaggctagagtgcagtggcatgatcttggctcactgcaacttccacctcctgggttcaagcaattctcctgcctcaacctcccaagcagctgggattatatgcgcccgccaccacgcctagctaatttttgtagagatgcggtttcactatgttggccgggctggtcttgatctcctgacctcaagtaatctgcccgcctcggcctcccaaagtgctgggattacatgtgtgaaccactgcacctggctaaaataaatttattttagattcagggggtacatgtgcaggtttgttacaagggtatattccaagatgctgaagtttgggctactaatgaccctgttgaccaagcagtgaacatagtacctgataggtagtttttcagcccttgccccgtagccgcttttggaatccccagtgtttattgttcccatctttgtgtccataagtacccagtgtttagctcccgcttataagtgagaacatggtgtttgattttctgtttctgtgttaatttgcttaagataatggcctccagctgcatccatgttgctgcagaggacatgattttgttcttttttatggctgcatagtatttcatggtgtgtatgtgccacattttctgtatcgtatttaccattgatgggcacctgggttgattccatgtctttgctgttgtgaatagtgctgcattgaacatgtgagggcaagtgtctttttggtagagcagtttattttcctttgggtagatagccagtaatgggattgctggttcaaatggtgattcagtttttagttctaagctttgtaatattccaccctgtttctattgtataggtttttttttttttttttttttttttgctatgcaggactactgcagtatctatagctttttagtcagtctctgtgatgaatatttacgttgtttccagtatttagccatttaaaataatgttgccgtgatgaataaccttgtgcacagagtatttcacattttgctgctgtaattttgggacatattcctagaagtgggattcctaggtcaaagggcaagtgcatctacaatttttctagatctggtcagattcccccaaagaggtttttgtattttgcattcccagcaaaatacaatcagcaatgatgattgctgtaccatgtgtttgtcaattgtgaatttgttcctagcatttagaaattgggagattctacttaaaaatccttatttccagtgtcttaaaaaaaaatcagagaatctgccttaatctacccatggcaacagttggctagagctgaataacagctgccccttttctaggtactctctaggttactgcagcccccacttggccattttacttacttttgctacctatcagtctcctatagacatgagtttttgacccctgctctaagtatggttttctgggtaggtagaatggagacggagagtttgggacttgaagacatagaggacaatttagattattatgttgtaagagccaccagtgtgaatgttgatgaccctaaagagggtggcagggaatatgacaaggggatattaggaacacaggattgaaatccttgaggaaagcacatgtcctagaggtccatggaagactaactgaaaaagaaggcatattttttgtggttgccatggtcctataccaaagaggttttgtgaaggcagcattacagaaggtctggaaaggaccagcagctgggagcagggagtgtgctaaagccctcctggtgtactcttcctgccttcccccatatactgaagtttgagaggctgggaaggtgcggaatgggaaaaggagcagctgcttatgttcagtttaacattctctgggtttctccatctaggtcttgagttcattctcttcctgtccttttggcttccttgtttaacctggtccctgtttcaggagagaagcctcatcagtgccaagtctgtgggaagaccttctctcagagtggaagcaggaatgtgcatatgagaaagcatcacctgcagctgggagcagctgggagtcaagagcaggagcaaactggtgaggagggtgggcatagtggaacgctgtactacttctagggttccaagagccaaatcactgtcctgcctgcttatctggaaattttctgattctgttttgggtagatagacctggtctcatttgatgggctgatggtttccaaacatagctatacagtagaatcatctgcaaccttttaaaatttatatactttttgttgcttgtccgcccaccaataaaacaaagcaatctgataaatgagataatttgcaagatcctagaatctgtagttttaaaagtttccctagtgagtttgatggtaactagtgctgatccatgaacaagcatttgagtaacacagtgataaaagatttccacatgggaagagtttaagctttttaatttttttagatggagtcttgctttgtcacccaggctggagtgcagtggcgccatctcgactcactgcaacctctgcctcctgggttcaagcagttttcctgcctcagcctcttgagtagctgggattataggcaggtgccaccacacctggctgatttttatatttttgtagagacggggtttcaccatgttggccaggctggaagcatattttatttaccagtcatgactacctttttgccctgcctgaaatttgataactctttttaattcttattccttcgccatatctttccttaattattccaaatattcctttctaaagatccacctatcctgtttcatatgctaattcttcagacgctgaacacaagcaaggatcattatcttctaaggcttactctttcccttctaggtttattcttgtttctcgaaagtaggtgaaatagggggtggcatcccatctataggttaacttctagtaatgaaagagaaggccagcttctgttgaaagaaaatgaggaccaggcacagccttgattgctgtatgtctctcacaacctattctctgtgcagctgagccactaatgggcagtagtttgcttgaagaggcttcagtacccagtaaaaacctggtgtctatgaattcccagcccagccttggtggagagtccttgaacctaccaaataccaattctatcctgggagttgatgatggtaagacttccaactctcctttatttgggaaaaatgggctgcaactaggggctaaggaatggggtgtctccacaatgtatgtttaaatctcatttcttatgagtagctgtggtatgagtagcatttgtgttcttattctctggggtggattagtcaatctgtttaaaaaaaaattttttttcattgaagataatataataaaagaataaaaagaaaaaattcttaagaaaaaattattcctaatcctagcatcctagcccaactatttctatctttcctattgcagttatggaagaacttaacttttagaacatttaaacagaacataaccaggttttacagaggagatataaagcaatttctccataccttcaaaagccatatcttctggagaaaagaaagggctctctaggaaacacttgctcctctctggtatagaaatttcacccaagttacacaggccaggccctcttctctgaaaaacttaacaggtgaggtcagcatgcatttattcagtgcctactgtgtgcctggcaccatgggaaagacaaaagtagaagatagggaccttaaaatccagttgagaagtgaaagcttacatatatatgggaaagaacattgagtacataaaagatagtatataagccaacattaaattatatggtacagcataaaatagttgtataatctagaaaacccctagaagaaacttctagttttttgagggctaagtggtatatccataaactatttccagaagattttcatggtgatcccactgatgaacatttagtaactgtgagaccctatgtttttgttactcatatcatattcagttagtaagtgctattagtatcattggcgactgcttttaagggtggctttggttcccttacctgcttgcctaagacttttgcctgtttcatacatagttttaattcagttaaaagtgctaacaccatattattctaaatcctgtattaagatttcttgcctttctattctctttttatattactttccaaattcactgataatgcttatcagacaggattaacttggaagaaatagaggaatgtgggaaatagaggaatagagttttagagttgttaatgagaggcttaatgatccagataggtgtatctctggattcacttttcattgaaacatttttctgttgcttcacagaggtgcttgctgaaggatccccacgttccctgtcttcagtgcctgatgtgacacatcacctggtgaccatgcagtcagggaggcaatcatatgaagtttctgtcttaactgcagtaaatccacaagaggtaaagtggtctcttgccctttgtttctgtgacaattcatgtggggaatgattgagaatttagaggacctgaaaaagtctccagtttccatttcctggaaactttggcacgaatattttcctttaaattaagcatgagttcagcttgaaaggagcctgagggttagggtcttaatgaattggagattggtctgtcacagctgggactcccggctttaaagttcttttatatacattccttgttgatgtctgttcccactattctctgccacactataacatgatacagcttttattttataaaagcacacagcaggcactgaataaatgcatgttgacctcacctgttaagttattcagagaagagggcctggcctgtgtaaattgggtgaaatttctgtaccagacaggagcaagtatttcctagagatccctttcttttctccagaaataacatcctggaaaaagtccctcagcaaaaaaggtagtaacaaaatcattaaacataattattgttcattctgctatggtcatatgtctgggagataccaggcttaaccaaatgggcatatacagatttgttcacttgcttgctaagtgagaaccattacctaaatcaatggtctccagcctttttggcaccagggaccggtttcgtttcacctcaggtcatcaggcattagattctgataaggagtgcgcaacctagatccctcaaatacacagttcacaatagggtttgcgctcctatgagaatctgttgctgctgctgatctgacaggagaggtggagctcaggtggtaatgcttgctcacctgctgctcacctcctgctgtgtggcctggttcctaacagggcacggaccagtgctggtccatggcctgggggttggtgacttctgatctaaacaaaaagggtttgaggttgagctaaagcactttcaactctgtagtttggctttgactcaggaaacagaatgcaaagtggtcagtgttttaaaatcttgggatatttcactggtcttgtgtcatgggagtggatgacttcatcacgagcacatttcatatgcataaacttttgtcaaaacctctgtagcttggatcctttcagtattacattagtttttatggtgctcttccgtcttttctcctcttttcttttctttttttttttgagacagagtctcgctccgttgcccaggctggagtgcagtggtgcgttctcggctcactgcaacctctgcctcccaggttcaagcgattctcctgcctcagcctcctgagtagctgctactacaggtgcgtgccaccacgcccagctaatttttttgtatttttagtagagacagggtttcaccatgttggccaggatggtctcgatctcttgacctcgtgatctgcccacctcagcctcccaaagtgctggaattacaggcgtgagccactgcacccagccttcttcttttcattataacctttataccacagtaagctgttgcatcattcaaatgactctgcatcctatattgaatattctccattattctctaatatctttgggggtacccaggattacagggttctgggcatacatgttaatgcttttgccaggacttttctttttattgttgtgacccattgtctttgcttatatttaaagtttttgatcttgatatcattggttctttcctaagtcactttttctggtaactgctttggaatgaatcatacatatctcacttacggagccttcctctttctcctccatttttcttgtcttcatttttctattttaaagcttttattatggaaattttcagagtacagtgagagagagactagtatgataaacccacgtcgaagtcgaagttacggctgagagcggcagctcacacctgtctcctaactttgacttttttttttttttgagatggagtcccactgtcgcccaggctggagtgcagtggtgcgatctcagctcactgcaacctccgcctctcaggttcaagcaattttcctgcctcagtctcctgaatagctgggattgcaggcacccactaccaaacccggctaatttttgtatttttattagagatagggttttaccctgttggccaggctagtctggaactcctgacctcaagtgatccacccgcctcagcctcccaaagtgctgggattacaggtgtgagctgccgcacttggccctaactttgacagttaactttatgccatgctcagtggcccatgcctataatcccagcactttcggaggtggaggcaagtggattgcttgaatccaggagttcaagaccagcctgggcaacataagacccctatctctacaaaaaatacaaaaaaaaattagcgggacgtggtggcatgtgcctgtggtcccaactactcgggaggctgaagtaggaggatcacctaagcctgggaggttgaggctacagtgagcagtgactgtgccactgtactccagcctgggtgacagagtgagaccctgtctcaaaagacaaaaacacaattaactttatactgttcttgttttatctctttcctccttttgttgttgttgctgtttatgatggagtatttcaaagcaaattgtaggcatgtcatttcttctgtatatatttcagtatatgcctctaacaagatacaatattaaaaactataatatctttaccacttcaaacaaagataacaataatttattgtattctctaatagtctatgttctttttttcctccccctcaaaaatatcttatggttggtttgctagagtcaggattcaaacaatgtctgtatattgcatttgtttaatacttctcttaattctctttataatagtccttttttttcagtatggttatctatttaaggaactgggttttttgttttgttttgtttttgtttttgttttgagactgagtctcactctgtggcccaggtcaagagtgcagtggcgtgatctcggctcactgcaacctctgcctcccaggttcaagcaattctcctgcctcaagtctcccaattagctgggattacaggcacctgccaccaagccccactaatttttgtatttttagtagagacgggcttttaccatgttggccaggctggtcccaaactcctgacctcaagtgatccacccacctcggcctcccaaagtgctgggattacaggcgtgagccactgcgcccggccaggaattgggagtttttgtcctaaagattttcccacattctggttttggctgattgactccttttgttaatattaatttctctatccactgtatttcctaaaaactggtagttacatttagaggtttgattagattcaatttttcccccttggcaagaatatttcataaatggtgctcttgcttcctattacatctcatcaggaggcacatgacaatcagttgatcttaagattgactggtaggttatgtaaaggttttggcaaccattactgatcatagcctagatctatttttcattaaggcctgcaaaatggtgatatcctaattctcttgttccctctgtgtttattatgattttttttttttttgaggtggagtcttgctatgttgcccaggctggagtgcagtggcaccatctcggctcactgcaacctctacctcccgaattcaagcggttcttctgcctcagcctcctgagtagctgggaccacaggcaggtgccaccacgcccggctgtatttttagtagagacggggtttcaccatgttggctaggctggtcttgaactcctgacctcaggtgatccacccgcctcagcctcccaaactgctgggattacaggcatgagccactgcgcccggcctaattgtgattcttttatacaagaaatacttctctcatcaaccatagggttgccctgaaatcgtcatttcaggaaaaccatctcgtttttcttttacctagtacacattattattcgagcaagccaacccacagcacctagccttttcttttttatatttgaaacctcagagcttttctctcctgtattggtaaatatctttaaaattctccacgttacccaaataaatgccagatgtatgaaaatttaattcattcagcggctagttaccacatacctagtatgtgtggggcattctgcagggtactagttatacaatagtaaacaaaatatagtctctgcctcaaatagcttataggcagaattaggaccttcataactatatatatggtgctggagttgcactgtacagtgaggttaggatgggagaagtgaccaattaatcaaccggcaattttttttttttttttaattttttagacggagtctcactctgttgcccaggctggagtgcagtggcacaatctcggcccactgcaacctctacctcctgggttcaagcaattctcatgtctcagactcctgagtagctgggattacaggcggccaccaccacacccagctaattttttgtatttttgatagagacggggtttcatcatgttggccaggctggtctcaaactcctgaccttaggtgatccacccgcctcggcctcccaaagtgctgggattacaggcatgagccactgcgcccagcctaaactggcagtttttaagtgtagtacgttccagtcttgggagaagtcagagaatatgttttagagtttaagttgagatgggagggatgaatagacatcatcttgatggagaagtaggagttggaagaatataacaggcagaaggacagcagtgtgtacaaaggtagaaggtaagggagtagagggcctctgtggaactgtaaatagggttgagtgtgatgggacatgggatgaagagatgggctggagaggaaaattgagagctggaaagataagctagtctcttgaaaggtcttacaaggcatgattttatcctgggggcactgaagaaccactgcatggtttgaagtagggtatgacgtgatcagatttgtgtattcagaatatccagtaatagtacatttgttccctagaacctaggcacaagtcttggaattggcctcagagcatccatgtataataggtgctgcccacttacagcctcagggagacagcataacacaggctgcctaaaaaacgaaagacctgggtgtggtggatcatgtctataatcccagcacttttggaagctaaggtgggaggatcacttgaggccagaagttcgagacaagcctggacaacagagcaagatcctgtctctacaaaaagttttttaaaaaattatccaggcatagtggtgcatacctgtagtcccagctactccagaggctgaggcaggaggatcaacttgagcccaggagttcgaggctgcagtgaactatgattgtcccactgcactccagcctgggagagatcctatcccaaaaatcaaacaaaaaaaggaagtatggtcttcactccaggaaggcacaaaacccagtttcataatggaagcccaaaacacttagaaaaatattaacaacacaaaagggaaagtgctgtcctggatgtagctcaaaatcctgcactgtcactttttcctcatgggtttttgtatatttacaaacataccctctgtctgagcctcacttcttttgtgttttcttcacttttagcattattctcctcctcgtatcacaggcatttccactgcttatcccctccttttctttcttcactagtttgggtcctccaataccttcagttttttttcaagctgtgaggccctcagggttccaggcttaggcctcttcctcatccaaggtctaggtaataacttgctatttctccagtgagtgaggaatgttgattccttaagaactttcaggtgccagagaaggtgaaggctatttgcaacacatgtccgtttcaccttttttggagatcaccaagcagtatggatcaaagtctactgatgaacttgtacttcctgtaaatatgctctaaaaataaaaatagcttatcaagaatatcagagaacaaccaaatctgttatcagaaattttcatgagaggctgggtgcggtggctagcactttgggaggctggggtgggaggatcaattgagcttaggagtttgagaccagcctgggcaacacaggaaaaccccatgtctacaaaaaataaaataaaattagctggtcctagctacttgggaggctgaggtgagaggatcacttgggcccaggaagttgaggctgtagtgagccatgatcttgccactacactgcagtctgggtgacagagccaaactctttaaaaaaaaaaaagaagaaaaattaccatgggaaaaatatcagaaactacaagtcacctctctggttcaggtggacaatctctattttccctcattattgtgccacatctgagaattagatggaggactcattgcacaggcccaactgggaaccccagggcttttgggctgtcatacttactaaaggtcattctaaatggggctccatatttacattgagttcacattcttgaactcaagaaccagtataagagattcccagctagttttgactacctcttagaatgctttccaaatcaaggcacaaatagaaattacaaattctataatatttctcataaagtacctttgctaagtttgactgcaaagatctggatcttggcctccattaaaaacacactattcttaactttcctgccacacagcatcaaatggaatatgtggtttctttaaggtttaattttttctttagattttgtttaaaagtctttaaattccatataattaaactcttaaaaaaaagtttttttggtggatacagtgtcttaccatgttgcccaggctgatctcaaatttctggcctcaagcaatcctcctccctcagcctcccaaagtggtggggttataggcatgagccatggtgcccagcccaatcattaaattctactgtataaattaccttcatccaaattaggatcaccaagtaaatacatctcaagctgaagaatttatttgattagattttaataagttgtttttgttctgaattttactgaattgttcccatctcatttttgtgtgtctgaaaacagtctcagtctgtcactcaggctggagtgcagtgcagtggcatgatcatagctcactgcacccttgaactcctgggctcaagtgatcctcctccctcagcctcccaagtagctaggactgcaagcatgtaccaccatgcttaatttttaaaattttcttgtagagacagggcctcactgtgttgcccaagtgggtctcaaatttctggtctcaagcagttctcctgcctcagcctcccaaagtgctgggattacagatgtgagccactgcacccagcctcattttgtttttatattaaaatggaagccaatttcattatttcaaaactcctttgaatagagccttgtgtatagagtagaatgttagtggtagttgccattatgcaggagtcccttaggcttatcattcttttaagcccaaggaacccctagatcttttatgagtgggtttcagctctcatatagtacttataaaggaacagctcctcttcctatttccttctcctacttgagccttcagcctatcctttggctgacacgtataagattttggttccaccatttctgttttccctgtaagttagagattcctctgtcactgactgatgagtaggtaactacacaaacaatagtttggcttatagactcagaatatttgattgccagagttaaaatgccagccttatgacatggctaaagtgctgtcccaaggccacagtgacatctatcagtccgtgcatgtgtttgctttggaagtagatagccttcattcacccctgtagtgcattctccatcctccactcttaatactttttactatgaattattttttctataattcaactgtaacctattctagatttgctttcaatcttttacagttactaaaccaaggagatttaactgaaagacggacatgagcgtgggtgctgactcctggaagagcaactctatctgatctcaaaatgcgtatactgggaacaggatgccttagcccacaacagaaccagaatgaatctttgaaggcacaagactctgcttttgccactcttcctctttcctggtatagaagatggatgtaggagagcttcttttctaactaccatctgatcagacaaggaatgaagcaatgactgtgggctgggaaactgtacctacctctcttcccactgcaaatttctgggatagaccaaaagtgaatttgattatgtgttggctgaagttcttcattctgactgttgaggggaggttttcctttgaagagttttcatcccagactcagctgtcttttcacatggatgaaataattcctgctaccaacaacagagcttcaccaggaagttgagttttcaagatgccttgttgctttgaagaagggagtgatgtcaattctcttgttacattctccctttagcaacctgagtaagagactctctgccactgggctgcaaaaaaataaattacttgaatctccccttggcccaggctgaggtactatcttgtcctatacacttctacttggtcactttgctttcttcgttaatggaacatacagtagcatgttaagagggatttcatgtttttgtttttttaaagttaaaaattacatgatgcagagattcaggttttcctttaaataaacaaaacagcccagtcagttctttggctcttgttttatacaaaatttgttttcttagagattaagaatttaatctttccctttaaaatagtgtattgatttacccttaggattccataccagtaaaactgaaccgaggagtcttaggaaacaacaagaacatcttcatttcttaagcccaggtgatagttactctgtcaccaccaaaaaagacgcatctgagaaaatggcaataaaaacagatacttctgaatttttcca

SEQ ID NO: 186 - DNA, 2792 bp, Homo sapiens
gtgtgtggacggaattcgggaccgactgacggccggccggcttcccggaactggaaggttacattgattacccacctagtacaacatcttacgggaagagcatagtatttcctagaggaatatgaacataacaggaaggtatcattggctctgaattaaatttgaacttgtcccctgaatagctacaggttttggaagctgaatcaatgttatcagatgagttagaatccaaaccagagctcctggtacagtttgttcagaatacgtccatcccattgggacaggggcttgtagaatcagaagctaaagatattacttgcttgtccctccttcccgtgactgaagcctcagaatgcagtcggctaatgttaccagaacctgttccatggagagaagaggatggaaagtcaggctgcagtgacttgaatgatactacaaatcattctaactcctccaaggaggtcccttcctcagctgttttgagaagccttcgggtgaatgtgggtccagacggagaggagacgagagctcagactgtacagaaatccccggagtttttgtccacttcagagtcttctagcttgttgcaagatctacagccaagtgatagcacttcttttattcttcttaacctaacaagagcaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcgaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaaggatgtggccgttcctttgctgagtattctagcctccgaaaacatctggtggttcactcaggagagaagcctcatcagtgccaagtctgtgggaagaccttctctcagagtggaagcaggaatgtgcatatgagaaagcatcacctgcagctgggagcagctgggagtcaagagcaggagcaaactgctgagccactaatgggcagtagtttgcttgaagaggcttcagtacccagtaaaaacctggtgtctatgaattcccagcccagccttggtggagagtccttgaacctaccaaataccaattctatcctgggagttgatgatgaggtgcttgctgaaggatccccacgttccctgtcttcagtgcctgatgtgacacatcacctggtgaccatgcagtcagggaggcaatcatatgaagtttctgtcttaactgcagtaaatccacaagagagtctcgctccgttgcccaggctggagtgcagtggtgcgttctcggctcactgcaacctctgcctcccaggttcaagcgattctcctgcctcagcctcctgagtagctgctactacagttactaaaccaaggagatttaactgaaagacggacatgagcgtgggtgctgactcctggaagagcaactctatctgatctcaaaatgcgtatactgggaacaggatgccttagcccacaacagaaccagaatgaatctttgaaggcacaagactctgcttttgccactcttcctctttcctggtatagaagatggatgtaggagagcttcttttctaactaccatctgatcagacaaggaatgaagcaatgactgtgggctgggaaactgtacctacctctcttcccactgcaaatttctgggatagaccaaaagtgaatttgattatgtgttggctgaagttcttcattctgactgttgaggggaggttttcctttgaagagttttcatcccagactcagctgtcttttcacatggatgaaataattcctgctaccaacaacagagcttcaccaggaagttgagttttcaagatgccttgttgctttgaagaagggagtgatgtcaattctcttgttacattctccctttagcaacctgagtaagagactctctgccactgggctgcaaaaaaataaattacttgaatctccccttggcccaggctgaggtactatcttgtcctatacacttctacttggtcactttgctttcttcgttaatggaacatacagtagcatgttaagagggatttcatgtttttgtttttttaaagttaaaaattacatgatgcagagattcaggttttcctttaaataaacaaaacagcccagtcagttctttggctcttgttttatacaaaatttgttttcttagagattaagaatttaatctttccctttaaaatagtgtattgatttacccttaggattccataccagtaaaactgaaccgaggagtcttaggaaacaacaagaacatcttcatttcttaagcccaggtgatagttactctgtcaccaccaaaaaagacgcatctgagaaaatggcaataaaaacagatacttctgaatttttcca

SEQ ID NO: 187 - DNA, 2623 bp, Homo sapiens
gtgtgtggacggaattcgggaccgactgacggccggccggcttcccggaactggaaggttacattgattacccacctagtacaacatcttacgggaagagcatagtatttcctagaggaatatgaacataacaggaaggtatcattggctctgaattaaatttgaacttgtcccctgaatagctacaggttttggaagctgaatcaatgttatcagatgagttagaatccaaaccagagctcctggtacagtttgttcagaatacgtccatcccattgggacaggggcttgtagaatcagaagctaaagatattacttgcttgtccctccttcccgtgactgaagcctcagaatgcagtcggctaatgttaccagatgatactacaaatcattctaactcctccaaggaggtcccttcctcagctgttttgagaagccttcgggtgaatgtgggtccagacggagaggagacgagagctcagactgtacagaaatccccggagtttttgtccacttcagagtcttctagcttgttgcaagatctacagccaagtgatagcacttcttttattcttcttaacctaacaagagcaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcgaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaaggatgtggccgttcctttgctgagtattctagcctccgaaaacatctggtggttcactcaggagagaagcctcatcagtgccaagtctgtgggaagaccttctctcagagtggaagcaggaatgtgcatatgagaaagcatcacctgcagctgggagcagctgggagtcaagagcaggagcaaactgctgagccactaatgggcagtagtttgcttgaagaggcttcagtacccagtaaaaacctggtgtctatgaattcccagcccagccttggtggagagtccttgaacctaccaaataccaattctatcctgggagttgatgatgaggtgcttgctgaaggatccccacgttccctgtcttcagtgcctgatgtgacacatcacctggtgaccatgcagtcagggaggcaatcatatgaagtttctgtcttaactgcagtaaatccacaagagttactaaaccaaggagatttaactgaaagacggacatgagcgtgggtgctgactcctggaagagcaactctatctgatctcaaaatgcgtatactgggaacaggatgccttagcccacaacagaaccagaatgaatctttgaaggcacaagactctgcttttgccactcttcctctttcctggtatagaagatggatgtaggagagcttcttttctaactaccatctgatcagacaaggaatgaagcaatgactgtgggctgggaaactgtacctacctctcttcccactgcaaatttctgggatagaccaaaagtgaatttgattatgtgttggctgaagttcttcattctgactgttgaggggaggttttcctttgaagagttttcatcccagactcagctgtcttttcacatggatgaaataattcctgctaccaacaacagagcttcaccaggaagttgagttttcaagatgccttgttgctttgaagaagggagtgatgtcaattctcttgttacattctccctttagcaacctgagtaagagactctctgccactgggctgcaaaaaaataaattacttgaatctccccttggcccaggctgaggtactatcttgtcctatacacttctacttggtcactttgctttcttcgttaatggaacatacagtagcatgttaagagggatttcatgtttttgtttttttaaagttaaaaattacatgatgcagagattcaggttttcctttaaataaacaaaacagcccagtcagttctttggctcttgttttatacaaaatttgttttcttagagattaagaatttaatctttccctttaaaatagtgtattgatttacccttaggattccataccagtaaaactgaaccgaggagtcttaggaaacaacaagaacatcttcatttcttaagcccaggtgatagttactctgtcaccaccaaaaaagacgcatctgagaaaatggcaataaaaacagatacttctgaatttttcca

SEQ ID NO: 188 - protein, 516 aa, Homo sapiens
MLSDELESKPELLVQFVQNTSIPLGQGLVESEAKDITCLSLLPVTEASECSRLMLPEPVPWREEDGKSGCSDLNDTTNHSNSSKEVPSSAVLRSLRVNVGPDGEETRAQTVQKSPEFLSTSESSSLLQDLQPSDSTSFILLNLTRAGLGSSAEHLVFVQDEAEDSGNDFLSSESTDSSIPWFLRVQELAHDSLIAATRAQLAKNAKTSSNGENVHLGSGDGQSKDSGPLPQVEKKLKCTVEGCDRTFVWPAHFKYHLKTHRNDRSFICPAEGCGKSFYVLQRLKVHMRTHNGEKPFMCHESGCGKQFTTAGNLKNHRRIHTGEKPFLCEAQGCGRSFAEYSSLRKHLVVHSGEKPHQCQVCGKTFSQSGSRNVHMRKHHLQLGAAGSQEQEQTAEPLMGSSLLEEASVPSKNLVSMNSQPSLGGESLNLPNTNSILGVDDEVLAEGSPRSLSSVPDVTHHLVTMQSGRQSYEVSVLTAVNPQESLAPLPRLECSGAFSAHCNLCLPGSSDSPASAS

SEQ ID NO: 189 - protein, 478 aa, Homo sapiens
MLSDELESKPELLVQFVQNTSIPLGQGLVESEAKDITCLSLLPVTEASECSRLMLPDDTTNHSNSSKEVPSSAVLRSLRVNVGPDGEETRAQTVQKSPEFLSTSESSSLLQDLQPSDSTSFILLNLTRAGLGSSAEHLVFVQDEAEDSGNDFLSSESTDSSIPWFLRVQELAHDSLIAATRAQLAKNAKTSSNGENVHLGSGDGQSKDSGPLPQVEKKLKCTVEGCDRTFVWPAHFKYHLKTHRNDRSFICPAEGCGKSFYVLQRLKVHMRTHNGEKPFMCHESGCGKQFTTAGNLKNHRRIHTGEKPFLCEAQGCGRSFAEYSSLRKHLVVHSGEKPHQCQVCGKTFSQSGSRNVHMRKHHLQLGAAGSQEQEQTAEPLMGSSLLEEASVPSKNLVSMNSQPSLGGESLNLPNTNSILGVDDEVLAEGSPRSLSSVPDVTHHLVTMQSGRQSYEVSVLTAVNPQELLNQGDLTERRT

SEQ ID NO: 190 - DNA, 2482 bp, Homo sapiens
gtgtgtggacggaattcgggaccgactgacggccggccggcttcccggaactggaaggttacattgattacccacctagtacaacatcttacgggaagagcatagtatttcctagaggaatatgaacataacaggaaggtatcattggctctgaattaaatttgaacttgtcccctgaatagctacaggttttggaagctgaatcaatgttatcagatgagttagaatccaaaccagagctcctggtacagtttgttcagaatacgtccatcccattgggacaggggcttgtagaatcagaagctaaagatattacttgcttgtccctccttcccgtgactgaagcctcagaatgcagtcggctaatgttaccagatgatactacaaatcattctaactcctccaaggaggtcccttcctcagctgttttgagaagccttcgggtgaatgtgggtccagacggagaggagacgagagctcagactgtacagaaatccccggagtttttgtccacttcagagtcttctagcttgttgcaagatctacagccaagtgatagcacttcttttattcttcttaacctaacaagagcaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcgaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaaggatgtggccgttcctttgctgagtattctagcctccgaaaacatctggtggttcactcaggagagaagcctcatcagtgccaagtctgtgggaagaccttctctcagagtggaagcaggaatgtgcatatgagaaagcatcacctgcagctgggagcagctgggagtcaagagcaggagcaaactgaggtgcttgctgaaggatccccacgttccctgtcttcagtgcctgatgtgacacatcacctggtgaccatgcagtcagggaggcaatcatatgaagtttctgtcttaactgcagtaaatccacaagagttactaaaccaaggagatttaactgaaagacggacatgagcgtgggtgctgactcctggaagagcaactctatctgatctcaaaatgcgtatactgggaacaggatgccttagcccacaacagaaccagaatgaatctttgaaggcacaagactctgcttttgccactcttcctctttcctggtatagaagatggatgtaggagagcttcttttctaactaccatctgatcagacaaggaatgaagcaatgactgtgggctgggaaactgtacctacctctcttcccactgcaaatttctgggatagaccaaaagtgaatttgattatgtgttggctgaagttcttcattctgactgttgaggggaggttttcctttgaagagttttcatcccagactcagctgtcttttcacatggatgaaataattcctgctaccaacaacagagcttcaccaggaagttgagttttcaagatgccttgttgctttgaagaagggagtgatgtcaattctcttgttacattctccctttagcaacctgagtaagagactctctgccactgggctgcaaaaaaataaattacttgaatctccccttggcccaggctgaggtactatcttgtcctatacacttctacttggtcactttgctttcttcgttaatggaacatacagtagcatgttaagagggatttcatgtttttgtttttttaaagttaaaaattacatgatgcagagattcaggttttcctttaaataaacaaaacagcccagtcagttctttggctcttgttttatacaaaatttgttttcttagagattaagaatttaatctttccctttaaaatagtgtattgatttacccttaggattccataccagtaaaactgaaccgaggagtcttaggaaacaacaagaacatcttcatttcttaagcccaggtgatagttactctgtcaccaccaaaaaagacgcatctgagaaaatggcaataaaaacagatacttctgaatttttcca

SEQ ID NO: 191 - protein, 431 aa, Homo sapiens
MLSDELESKPELLVQFVQNTSIPLGQGLVESEAKDITCLSLLPVTEASECSRLMLPDDTTNHSNSSKEVPSSAVLRSLRVNVGPDGEETRAQTVQKSPEFLSTSESSSLLQDLQPSDSTSFILLNLTRAGLGSSAEHLVFVQDEAEDSGNDFLSSESTDSSIPWFLRVQELAHDSLIAATRAQLAKNAKTSSNGENVHLGSGDGQSKDSGPLPQVEKKLKCTVEGCDRTFVWPAHFKYHLKTHRNDRSFICPAEGCGKSFYVLQRLKVHMRTHNGEKPFMCHESGCGKQFTTAGNLKNHRRIHTGEKPFLCEAQGCGRSFAEYSSLRKHLVVHSGEKPHQCQVCGKTFSQSGSRNVHMRKHHLQLGAAGSQEQEQTEVLAEGSPRSLSSVPDVTHHLVTMQSGRQSYEVSVLTAVNPQELLNQGDLTERRT

SEQ ID NO: 192 - DNA, 2404 bp, Homo sapiens
gtgtgtggacggaattcgggaccgactgacggccggccggcttcccggaactggaaggttacattgattacccacctagtacaacatcttacgggaagagcatagtatttcctagaggaatatgaacataacaggaaggtatcattggctctgaattaaatttgaacttgtcccctgaatagctacaggttttggaagctgaatcaatgttatcagatgagttagaatccaaaccagagctcctggtacagtttgttcagaatacgtccatcccattgggacaggggcttgtagaatcagaagctaaagatattacttgcttgtccctccttcccgtgactgaagcctcagaatgcagtcggctaatgttaccaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcgaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaaggatgtggccgttcctttgctgagtattctagcctccgaaaacatctggtggttcactcaggagagaagcctcatcagtgccaagtctgtgggaagaccttctctcagagtggaagcaggaatgtgcatatgagaaagcatcacctgcagctgggagcagctgggagtcaagagcaggagcaaactgctgagccactaatgggcagtagtttgcttgaagaggcttcagtacccagtaaaaacctggtgtctatgaattcccagcccagccttggtggagagtccttgaacctaccaaataccaattctatcctgggagttgatgatgaggtgcttgctgaaggatccccacgttccctgtcttcagtgcctgatgtgacacatcacctggtgaccatgcagtcagggaggcaatcatatgaagtttctgtcttaactgcagtaaatccacaagagttactaaaccaaggagatttaactgaaagacggacatgagcgtgggtgctgactcctggaagagcaactctatctgatctcaaaatgcgtatactgggaacaggatgccttagcccacaacagaaccagaatgaatctttgaaggcacaagactctgcttttgccactcttcctctttcctggtatagaagatggatgtaggagagcttcttttctaactaccatctgatcagacaaggaatgaagcaatgactgtgggctgggaaactgtacctacctctcttcccactgcaaatttctgggatagaccaaaagtgaatttgattatgtgttggctgaagttcttcattctgactgttgaggggaggttttcctttgaagagttttcatcccagactcagctgtcttttcacatggatgaaataattcctgctaccaacaacagagcttcaccaggaagttgagttttcaagatgccttgttgctttgaagaagggagtgatgtcaattctcttgttacattctccctttagcaacctgagtaagagactctctgccactgggctgcaaaaaaataaattacttgaatctccccttggcccaggctgaggtactatcttgtcctatacacttctacttggtcactttgctttcttcgttaatggaacatacagtagcatgttaagagggatttcatgtttttgtttttttaaagttaaaaattacatgatgcagagattcaggttttcctttaaataaacaaaacagcccagtcagttctttggctcttgttttatacaaaatttgttttcttagagattaagaatttaatctttccctttaaaatagtgtattgatttacccttaggattccataccagtaaaactgaaccgaggagtcttaggaaacaacaagaacatcttcatttcttaagcccaggtgatagttactctgtcaccaccaaaaaagacgcatctgagaaaatggcaataaaaacagatacttctgaatttttcca

SEQ ID NO: 193 - DNA, 2716 bp, Homo sapiens
gtgtgtggacggaattcgggaccgactgacggccggccggcttcccggaactggaaggttacattgattacccacctagtacaacatcttacgggaagagcatagtatttcctagaggaatatgaacataacaggaaggtatcattggctctgaattaaatttgaacttgtcccctgaatagctacaggttttggaagctgaatcaatgttatcagatgagttagaatccaaaccagagctcctggtacagtttgttcagaatacgtccatcccattgggacaggggcttgtagaatcagaagctaaagatattacttgcttgtccctccttcccgtgactgaagcctcagaatgcagtcggctaatgttaccagatgatactacaaatcattctaactcctccaaggaggtcccttcctcagctgttttgagaagccttcgggtgaatgtgggtccagacggagaggagacgagagctcagactgtacagaaatccccggagtttttgtccacttcagagtcttctagcttgttgcaagatctacagccaagtgatagcacttcttttattcttcttaacctaacaagagcaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcgaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaaggatgtggccgttcctttgctgagtattctagcctccgaaaacatctggtggttcactcaggagagaagcctcatcagtgccaagtctgtgggaagaccttctctcagagtggaagcaggaatgtgcatatgagaaagcatcacctgcagctgggagcagctgggagtcaagagcaggagcaaactgctgagccactaatgggcagtagtttgcttgaagaggcttcagtacccagtaaaaacctggtgtctatgaattcccagcccagccttggtggagagtccttgaacctaccaaataccaattctatcctgggagttgatgatgaggtgcttgctgaaggatccccacgttccctgtcttcagtgcctgatgtgacacatcacctggtgaccatgcagtcagggaggcaatcatatgaagtttctgtcttaactgcagtaaatccacaagaggtggagtcttgctatgttgcccaggctggagtgcagtggcaccatctcggctcactgcaacctctacctcccgaattcaagcggttcttctgcctcagcctcctgagtagctgggaccacagttactaaaccaaggagatttaactgaaagacggacatgagcgtgggtgctgactcctggaagagcaactctatctgatctcaaaatgcgtatactgggaacaggatgccttagcccacaacagaaccagaatgaatctttgaaggcacaagactctgcttttgccactcttcctctttcctggtatagaagatggatgtaggagagcttcttttctaactaccatctgatcagacaaggaatgaagcaatgactgtgggctgggaaactgtacctacctctcttcccactgcaaatttctgggatagaccaaaagtgaatttgattatgtgttggctgaagttcttcattctgactgttgaggggaggttttcctttgaagagttttcatcccagactcagctgtcttttcacatggatgaaataattcctgctaccaacaacagagcttcaccaggaagttgagttttcaagatgccttgttgctttgaagaagggagtgatgtcaattctcttgttacattctccctttagcaacctgagtaagagactctctgccactgggctgcaaaaaaataaattacttgaatctccccttggcccaggctgaggtactatcttgtcctatacacttctacttggtcactttgctttcttcgttaatggaacatacagtagcatgttaagagggatttcatgtttttgtttttttaaagttaaaaattacatgatgcagagattcaggttttcctttaaataaacaaaacagcccagtcagttctttggctcttgttttatacaaaatttgttttcttagagattaagaatttaatctttccctttaaaatagtgtattgatttacccttaggattccataccagtaaaactgaaccgaggagtcttaggaaacaacaagaacatcttcatttcttaagcccaggtgatagttactctgtcaccaccaaaaaagacgcatctgagaaaatggcaataaaaacagatacttctgaatttttcca

SEQ ID NO: 194 - protein, 9 aa, Unknown
  Description of Unknown “LAGLIDADG” family peptide motif sequence
LAGLIDADG